(19) United States
(12) Patent Application Publication
(10) Pub. No.: US 2025/0000977 A1
(43) Pub. Date: Jan. 2, 2025

(54) BCMA-TARGETED CAR-T CELL THERAPY FOR MULTIPLE MYELOMA

(71) Applicants: LEGEND BIOTECH USA INC., Somerset, NJ (US); JANSSEN BIOTECH, INC., Horsham, PA (US)

(72) Inventors: Nitin PATEL, Somerset, NJ (US); Lida PACAUD, Somerset, NJ (US); Yuhong QIU, Somerset, NJ (US); Nikoletta LENDVAI, West Windsor, NJ (US); William DERAEDT, Schelle (BE); Jordan Mark SCHECTER, Livingston, NJ (US); Ana Rute DE ASCENSAO SLAUGHTER, Boudry (CH); Carolina LONARDI, City of Panama (PA)

(73) Assignees: LEGEND BIOTECH USA INC., Somerset, NJ (US); JANSSEN BIOTECH, INC., Horsham, PA (US)

(21) Appl. No.: 18/639,682

(22) Filed: Apr. 18, 2024

Related U.S. Application Data
(60) Provisional application No. 63/504,184, filed on May 24, 2023.
(60) Provisional application No. 63/497,185, filed on Apr. 19, 2023.

(51) Int. Cl. A61K 39/00; A61P 35/00; C07K 14/725; C07K 16/28

(52) U.S. Cl. CPC A61K 39/464438; A61K 39/4611; A61K 39/4631; A61P 35/00; C07K 14/7051; C07K 16/2875; C07K 2317/569

(57) ABSTRACT

Provided herein are methods of treating a subject who has multiple myeloma and has received one to three prior treatment(s). Infusions of chimeric antigen receptor (CAR)-T cells comprising a CAR capable of specifically binding to an epitope of BCMA are administered to the subject.

### CROSS-REFERENCE TO RELATED APPLICATIONS

This application claims benefit of priority of U.S. Provisional Patent Application Nos. 63/497,185 filed on Apr. 19, 2023, and 63/504,184 filed on May 24, 2023, the disclosure of each of which is incorporated by reference herein in its entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “14651-066-999_SEQ_LISTING.xml”, was created on Aug. 24, 2023, and is 28,450 bytes in size.

### BACKGROUND

Multiple myeloma is a neoplasm of plasma cells that is aggressive. Multiple myeloma is considered to be a B-cell neoplasm that proliferates uncontrollably in the bone marrow. Symptoms include one or more of hypercalcemia, renal insufficiency, anemia, bony lesions, bacterial infections, hyperviscosity and amyloidosis. Multiple myeloma is still considered to be an almost incurable disease, despite availability of new therapies that include proteasome inhibitors, immunomodulatory drugs, and monoclonal antibodies that have significantly improved patient outcomes. Because most patients will either relapse or become refractory to treatment, there is an ongoing need for new therapies for multiple myeloma. Particularly, patients with multiple myeloma who have received 1-3 prior lines of therapies and are lenalidomide-refractory have poor outcomes and progress rapidly through available therapies, highlighting the need for new, safe and effective treatment regimens for use in these earlier-line settings.

### SUMMARY OF THE DISCLOSURE

In one aspect, provided herein is a method of treating a subject, comprising administering to the subject a dose of T cells comprising a chimeric antigen receptor (CAR) comprising: (a) an extracellular antigen binding domain capable of specifically binding to an epitope of B-cell maturation antigen (BCMA), (b) a transmembrane domain, and (c) an intracellular signaling domain. In some embodiments, the subject has multiple myeloma, has received one to three prior lines of therapies, including a therapy with an immunomodulatory drug (IMiD), and is refractory to the IMiD. In some embodiments, the subject has a high-risk feature, and optionally the high-risk feature is a cytogenetic abnormality, International Staging System (ISS) stage III, and/or soft tissue plasmacytomas.

In one aspect, provided herein is a method of selectively treating a subject, comprising: (1) determining whether the subject has a high-risk feature, the high-risk feature being a cytogenetic abnormality, International Staging System (ISS) stage III, and/or soft tissue plasmacytomas; and (2) administering to the subject who is determined to have the high-risk feature in step (1) a dose of T cells comprising a chimeric antigen receptor (CAR) comprising: (a) an extracellular antigen binding domain capable of specifically binding to an epitope of BCMA, (b) a transmembrane domain, and (c) an intracellular signaling domain. In some embodiments, the subject has multiple myeloma, has received one to three prior lines of therapies, including a therapy with an IMiD, and is refractory to the IMiD.

In one aspect, provided herein is a method of selectively treating a subject, comprising administering to the subject who has been determined to have a high-risk feature a dose of T cells comprising a chimeric antigen receptor (CAR) comprising: (a) an extracellular antigen binding domain capable of specifically binding to an epitope of BCMA, (b) a transmembrane domain, and (c) an intracellular signaling domain. In some embodiments, the high-risk feature is a cytogenetic abnormality, International Staging System (ISS) stage III, and/or soft tissue plasmacytomas. In some embodiments, the subject has multiple myeloma, has received one to three prior lines of therapies, including a therapy with an IMiD, and is refractory to the IMiD.

In some embodiments of the various methods or aspects provided herein, the IMiD is lenalidomide.

In some embodiments of the various methods or aspects provided herein, the high-risk feature is a cytogenetic abnormality. In some embodiments, the cytogenetic abnormality is a high-risk cytogenetic abnormality. In some embodiments, the subject has one or more high-risk cytogenetic abnormality selected from a group comprising Gain/amp(1q), del(17p), t(4;14), t(14;16), or any combination thereof. In some embodiments, the cytogenetic abnormality comprises Gain/amp(1q). In some embodiments, the cytogenetic abnormality comprises del(17p). In some embodiments, the cytogenetic abnormality comprises t(4;14). In some embodiments, the cytogenetic abnormality comprises t(14;16). In some embodiments, the subject has at least two cytogenetic abnormalities. In some embodiments, the subject has at least three cytogenetic abnormalities. In some embodiments, the subject has at least four cytogenetic abnormalities. In some embodiments, the subject has at least five cytogenetic abnormalities. In some embodiments, the subject has at least six cytogenetic abnormalities. In some embodiments, the subject has at least seven cytogenetic abnormalities. In other embodiments, the cytogenetic abnormality is a standard-risk cytogenetic abnormality. In other embodiments, the high-risk feature is International Staging System (ISS) stage III. In yet other embodiments, the high-risk feature is soft tissue plasmacytomas.

In some embodiments of the various methods or aspects provided herein, the subject has received one prior line of therapy. In some embodiments, the subject has received two prior lines of therapy. In some embodiments, the subject has received three prior lines of therapy.

In some embodiments of the various methods or aspects provided herein, the, or one of the, prior lines of therapy comprises an IMiD. In some embodiments, the IMiD is or comprises pomalidomide. In some embodiments the IMiD is or comprises lenalidomide. In some embodiments the subject has received prior treatment comprising a combination of lenalidomide and pomalidomide.

In some embodiments of the various methods or aspects provided herein, the, or one of the, prior lines of therapy comprises an anti-CD38 antibody. In some embodiments, the anti-CD38 antibody is daratumumab and/or isatuximab.

In some embodiments of the various methods or aspects provided herein, the, or one of the, prior lines of therapy comprises a proteasome inhibitor. In some embodiments, the proteasome inhibitor is bortezomib, carfilzomib, ixazomib, or any combination thereof.

In some embodiments of the various methods or aspects provided herein, the subject has further received a bridging therapy. In certain embodiments, the bridging therapy is of the physician's choice. In some embodiments, the bridging therapy comprises pomalidomide, bortezomib, dexamethasone, daratumumab, or any combination thereof. In some embodiments, the bridging therapy comprises pomalidomide, bortezomib and dexamethasone. In other embodiments, the bridging therapy comprises daratumumab, pomalidomide and dexamethasone. In some embodiments, the subject has received the bridging therapy from about every 20 days to about every 30 days. In some embodiments, the subject has received the bridging therapy about every 21 days. In some embodiments, the subject has received the bridging therapy about every 28 days. In some embodiments, the subject has received at least one, two, three, four, or more cycles of bridging therapies.

In some embodiments of the various methods or aspects provided herein, the subject has further received a lymphodepletion therapy. In some embodiments, the lymphodepletion therapy comprises cyclophosphamide and/or fludarabine daily. In some embodiments, the lymphodepletion therapy comprises cyclophosphamide and fludarabine daily. In some embodiments, the lymphodepletion therapy comprises cyclophosphamide at a concentration of about 300 mg/m² and fludarabine at a concentration of about 30 mg/m² daily for 3 days.

In some embodiments of the various methods or aspects provided herein, the dose of the T cells is 0.5-1.0×10⁶ cells/kg of body weight of the subject. In preferred embodiments, the dose of the T cells is about 0.75×10⁶ cells/kg of body weight of the subject. In some embodiments, the method comprises administering the dose of the T cells about 5 to about 7 days after the start of the lymphodepletion therapy. Preferably, the dose is administered as a single infusion.

In some embodiments of the various methods or aspects provided herein, the method is effective in obtaining an overall response in the subject after administering to the subject the dose of the T cells. In some embodiments, the method is effective in obtaining the overall response at a rate of about 70% to about 100%. In some embodiments, the method is effective in obtaining the overall response at a rate of about 74%. In some embodiments, the method is effective in obtaining the overall response at a rate of about 84.6%. In some embodiments, the method is effective in obtaining the overall response at a rate of about 99.4%.

In some embodiments of the various methods or aspects provided herein, the overall response comprises, in order from best to worst: (1) a stringent complete response; (2) a complete response; (3) a very good partial response; (4) a partial response; or (5) a minimal response. In some embodiments, the overall response is a stringent complete response. In some embodiments, the method is effective in obtaining the stringent complete response at a rate of about 40% to about 90%, about 50% to about 80%, about 58.2%, or about 68.8%. In other embodiments, the overall response is a complete response. In some embodiments, the method is effective in obtaining the complete response at a rate of about 10% to about 20%. In some embodiments, the method is effective in obtaining the complete response at a rate of about 14.9% or about 17.6%. In some embodiments, the overall response is a very good partial response or a partial response. In some embodiments, the method is effective in obtaining a stringent complete response or a complete response at a rate of about 70% to about 90%. In some embodiments, the method is effective in obtaining a stringent complete response or a complete response at a rate of about 73.1% or about 86.4%. In some embodiments, the method is effective in obtaining a stringent complete response, a complete response, or a very good partial response at a rate of about 80% to about 100%. In some embodiments, the method is effective in obtaining a stringent complete response, a complete response, or a very good partial response at a rate of about 81.3% or about 96.0%. In some embodiments, the method is effective in obtaining a minimal response. In some embodiments, the method is effective in further obtaining minimal residual disease negative. In some embodiments, the method is effective in obtaining minimal residual disease negative at a rate of about 50% to about 80%. In some embodiments, the method is effective in obtaining minimal residual disease negative at a rate of about 60.6%. In some embodiments, the method is effective in obtaining minimal residual disease negative at a rate of about 71.6%

In some embodiments of the various methods or aspects provided herein, the method is effective in further obtaining 12-month progression-free survival for at least about 60 to about 100% of subjects. In some embodiments, the method is effective in further obtaining 12-month progression-free survival for at least about 69.4 to about 81.1% of subjects. In some embodiments, the method is effective in further obtaining 12-month progression-free survival for at least about 84.1 to about 93.4% of subjects. In some embodiments, the method is effective in obtaining 12-month progression-free survival for at least about 75.9% of subjects. In some embodiments, the method is effective in obtaining 12-month progression-free survival for at least about 89.7% of subjects.

In some embodiments of the various methods or aspects provided herein, time to first overall response or first minimal response ranges about 0.9 to about 11.1 months. In some embodiments, the time to the first overall response or the first minimal response is at a median of about 2.1 months.

In some embodiments of the various methods or aspects provided herein, time to best overall response or best minimal response is about 1.1 to about 18.6 months. In some embodiments, the time to the best overall response or the best minimal response is at a median of about 6.4 months. In some embodiments, the time to the best overall response or the best minimal response is at a median of about 6.5 months.

In some embodiments of the various methods or aspects provided herein, the method further comprises treating the subject for an adverse event after administering the dose of the T cells. In some embodiments, the method comprises administering a treatment to the subject to alleviate the adverse event. In some embodiments, the adverse event comprises a hematologic adverse event, a nonhematologic adverse event, a treatment-emergent adverse event, or any combination thereof. In some embodiments, the nonhematologic adverse event comprises an infection and/or a nonhematologic adverse event other than an infection. In some embodiments, the adverse event comprises neutropenia, thrombocytopenia, anemia, lymphopenia, an upper respiratory tract infection, nasopharyngitis, sinusitis, rhinitis, tonsillitis, pharyngitis, laryngitis, pharyngotonsillitis, COVID-19, COVID-19 pneumonia, asymptomatic COVID-19, neutropenic sepsis, progressive multifocal leukoencephalpathy, septic shock, respiratory failure, pulmonary embolism, a lower respiratory tract/lung infection, pneumonia, bronchitis, nausea, hypogammaglobulinemia, diarrhea, fatigue, headache, constipation, hypokalemia, asthenia, peripheral edema, decreased appetite, peripheral sensory neuropathy, back pain, arthralgia, pyrexia, dyspnea, insomnia, or any combination thereof. In some embodiments, the adverse event is a Grade 3/4 adverse event. In some embodiments, the adverse event lasts longer than about 30 days or about 60 days.

In some embodiments of the various methods or aspects provided herein, the method further comprises treating the subject for a second primary malignancy after administering the dose of the T cells. In some embodiments, the method comprises administering a treatment to the subject to alleviate the second primary malignancy. In some embodiments, the second primary malignancy comprises a cutaneous/non-invasive malignancy, a hematologic malignancy, a non-cutaneous/invasive malignancy, or any combination thereof. In some embodiments, the second primary malignancy comprises basal cell carcinoma, Bowen's disease, lip squamous cell carcinoma, malignant melanoma, malignant melanoma in situ, squamous cell carcinoma of skin, acute myeloid leukemia, a myelodysplastic syndrome, peripheral T-cell lymphoma, angiosarcoma, invasive lobular breast carcinoma, pleomorphic malignant fibrous histiocytoma, renal cell carcinoma, tonsil cancer, or any combination thereof.

In some embodiments of the various methods or aspects provided herein, the adverse event or the second primary malignancy occurs in the subject at a rate comparable to a rate of a same adverse event or a same second primary malignancy occurring in a subject undergoing a standard of care.

In some embodiments of the various methods or aspects provided herein, the method further comprises treating the subject for a CAR-T-associated adverse event after administering the dose of the T cells. In some embodiments, the method comprises administering a treatment to the subject to alleviate the CAR-T-associated adverse event. In some embodiments, the CAR-T-associated adverse event comprises a cytokine release syndrome (CRS) and/or neurotoxicity.

In some embodiments of the various methods or aspects provided herein, the CAR-T-associated adverse event is a CRS. In some embodiments, the CRS occurs in the subject at a rate of about 60% to about 90%. In some embodiments, the CRS occurs in the subject at a rate of about 76.1%. In some embodiments, maximum toxicity grade of the CRS is Grade 1, Grade 2 or Grade 3. In some embodiments, the maximum toxicity grade of the CRS is Grade 1. In some embodiments, the maximum toxicity grade of Grade 1 in the subject occurs at a rate of about 52.8%. In some embodiments, the maximum toxicity grade of the CRS is Grade 2. In some embodiments, the maximum toxicity grade of Grade 2 in the subject occurs at a rate of about 22.2%. In some embodiments, the maximum toxicity grade of the CRS is Grade 3. In some embodiments, the maximum toxicity grade of Grade 3 in the subject occurs at a rate of about 1.1%. In some embodiments, time to first onset of the CRS ranges from about 1 to about 23 days. In some embodiments, the time to the first onset of the CRS is at a median of about 8 days. In some embodiments, duration of the CRS ranges from about 1 to about 17 days. In some embodiments, the duration of the CRS is at a median of about 3 days. In some embodiments of the various methods or aspects provided herein, the treatment of the adverse event comprises tocilizumab, oxygen, a corticosteroid, a vasopressor, or any combination thereof.

In some embodiments of the various methods or aspects provided herein, the CAR-T-associated adverse event is neurotoxicity. In some embodiments, the neurotoxicity comprises an immune effector cell-associated neurotoxicity syndrome or associated symptom, movement and neurocognitive neurotoxicity, treatment-emergent adverse event of neurotoxicity, a non-immune effector cell-associated neurotoxicity syndrome or associated symptom, or any combination thereof. In some embodiments, the neurotoxicity is an immune effector cell-associated neurotoxicity syndrome or associated symptom.

In some embodiments of the various methods or aspects provided herein, the immune effector cell-associated neurotoxicity syndrome or associated symptom in the subject occurs at a rate of about 4.5%. In some embodiments, maximum toxicity grade of the immune effector cell-associated neurotoxicity syndrome or associated symptom is Grade 1 or Grade 2. In some embodiments, maximum toxicity grade of the immune effector cell-associated neurotoxicity syndrome or associated symptom is Grade 1. In some embodiments, the maximum toxicity grade of Grade 1 in the subject occurs at a rate of about 3.4%. In other embodiments, maximum toxicity grade of the immune effector cell-associated neurotoxicity syndrome or associated symptom is Grade 2. In some embodiments, the maximum toxicity grade of Grade 2 in the subject occurs at a rate of about 1.1%. In some embodiments, time to onset of the immune effector cell-associated neurotoxicity syndrome or associated symptom ranges from about 6 to about 15 days. In some embodiments, the time to the onset of the immune effector cell-associated neurotoxicity syndrome or associated symptom is at a median of about 9.5 days. In some embodiments, duration of the immune effector cell-associated neurotoxicity syndrome or associated symptom ranges from about 1 to about 6 days. In some embodiments, the duration of the immune effector cell-associated neurotoxicity syndrome or associated symptom is at a median of about 2 days. In some embodiments, the treatment of the adverse event comprises a corticosteroid and/or tocilizumab.

In some embodiments of the various methods or aspects provided herein, the neurotoxicity is a CAR-T cell neurotoxicity. In some embodiments, the CAR-T cell neurotoxicity in the subject occurs at a rate of about 17.0%. In some embodiments, the CAR-T cell neurotoxicity comprises Grade 3/4 neurotoxicity, Grade 5 neurotoxicity, cranial nerve palsy, peripheral neuropathy, a movement and neurocognitive treatment-emergent adverse event, or any combination thereof. In some embodiments, the CAR-T cell neurotoxicity is Grade 3/4 neurotoxicity that occurs in the subject at a rate of about 2.3%. In some embodiments, the CAR-T cell neurotoxicity is Grade 5 neurotoxicity. In some embodiments, the CAR-T cell neurotoxicity is cranial nerve palsy. In some embodiments, the cranial nerve palsy in the subject occurs at a rate of about 9.1%. In some embodiments, the cranial nerve palsy is Grade 2 or Grade 3 cranial nerve palsy. In some embodiments, the cranial nerve palsy is Grade 2 cranial nerve palsy that occurs in the subject at a rate of about 8.0%. In some embodiments, the cranial nerve palsy is Grade 3 cranial nerve palsy that occurs in the subject at a rate of about 1.1%. In some embodiments, time to onset of the cranial nerve palsy after administering the dose of the T cells to the subject ranges from about 17 days to about 60 days. In some embodiments, the time to onset of the cranial nerve palsy after administering the dose of the T cells to the subject is at a median of about 21 days. In some embodiments, the cranial nerve palsy affects cranial nerve III, V, or VII. In some embodiments, duration of the cranial nerve palsy ranges from about 15 days to about 262 days. In some embodiments, duration of the cranial nerve palsy is at a median of about 77 days. In some embodiments, the treatment comprises a corticosteroid. In some embodiments, the CAR-T cell neurotoxicity is peripheral neuropathy. In some embodiments, the peripheral neuropathy in the subject occurs at a rate of about 2.8%. In some embodiments, the peripheral neuropathy is Grade 1. In some embodiments, the Grade 1 peripheral neuropathy occurs at a rate of about 1.1%. In some embodiments, the peripheral neuropathy is Grade 2. In some embodiments, the Grade 2 peripheral neuropathy occurs at a rate of about 1.1%. In some embodiments, the peripheral neuropathy is Grade 3. In some embodiments, the Grade 3 peripheral neuropathy occurs at a rate of about 0.6%. In some embodiments, the CAR-T cell neurotoxicity is a movement and neurocognitive treatment-emergent adverse event. In some embodiments, the movement and neurocognitive treatment-emergent adverse event is Grade 1. In some embodiments, the Grade 1 movement and neurocognitive treatment-emergent adverse event in the subject occurs at a rate of about 0.6%.

In some embodiments of the various methods or aspects provided herein, CD3+ cells comprising the CAR in the blood of the subject peak at a median of about 13 days after administering the T cells to the subject. In some embodiments, the CD3+ cells comprising the CAR in the blood of the subject peak at a mean concentration of about 1523 cells/μL. In some embodiments, CD3+ cells comprising the CAR in the blood of the subject remain detectable from about 13 days to about 631 days after administering the T cells to the subject. In some embodiments, the CD3+ cells comprising the CAR in the blood of the subject remain detectable at a median of about 57 days after administering the T cells to the subject. In some embodiments, AUC₀₋₂₈ of CD3+ cells comprising the CAR in the blood of the subject is at a mean value of about 12,504 cells/μL.

In some embodiments of the various methods or aspects provided herein, the first VHH domain comprises a CDR1, a CDR2, and a CDR3 of the VHH domain comprising the amino acid sequence of SEQ ID NO: 2, and the second VHH domain comprises a CDR1, a CDR2, and a CDR3 of the VHH domain comprising the amino acid sequence of SEQ ID NO: 4. In some embodiments, the first VHH domain comprises a CDR1 comprising the amino acid sequence of SEQ ID NO: 18, a CDR2 comprising the amino acid sequence of SEQ ID NO: 19, a CDR3 comprising the amino acid sequence of SEQ ID NO: 20, and the second VHH domain comprises a CDR1 comprising the amino acid sequence of SEQ ID NO: 21, a CDR2 comprising the amino acid sequence of SEQ ID NO: 22, and a CDR3 comprising the amino acid sequence of SEQ ID NO: 23. In some embodiments, the first VHH domain comprises the amino acid sequence of SEQ ID NO: 2 and the second VHH domain comprises the amino acid sequence of SEQ ID NO: 4. In some embodiments, the first VHH domain is at the N-terminus of the second VHH domain. In other embodiments, the first VHH domain is at the C-terminus of the second VHH domain. In some embodiments, the first VHH domain is linked to the second VHH domain via a linker comprising the amino acid sequence of SEQ ID NO: 3. In some embodiments, the transmembrane domain is derived from a molecule selected from the group consisting of CD8a, CD4, CD28, CD137, CD80, CD86, CD152 and PD1. In some embodiments, the transmembrane domain is derived from CD8a and comprises the amino acid sequence of SEQ ID NO: 6. In some embodiments, the intracellular signaling domain comprises a primary intracellular signaling domain of an immune effector cell. In some embodiments, the primary intracellular signaling domain is derived from CD3ζ comprising the amino acid sequence of SEQ ID NO: 8. In some embodiments, the intracellular signaling domain comprises a co-stimulatory signaling domain. In some embodiments, the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 and any combination thereof. In some embodiments, the co-stimulatory signaling domain comprises a cytoplasmic domain of CD137 comprising the amino acid sequence of SEQ ID NO: 7. In some embodiments, the CAR further comprises a hinge domain located between the C-terminus of the extracellular antigen binding domain and the N-terminus of the transmembrane domain. In some embodiments, the hinge domain is derived from CD8a comprising the amino acid sequence of SEQ ID NO: 5. In some embodiments, the CAR further comprises a signal peptide located at the N-terminus of the polypeptide. In some embodiments, the signal peptide is derived from CD8a comprising the amino acid sequence of SEQ ID NO: 1. In some embodiments, the CAR comprises the amino acid sequence of SEQ ID NO: 17.

In some aspects, provided herein are methods of treating a subject with multiple myeloma, the method comprising administering to the subject a dose of T cells comprising a chimeric antigen receptor (CAR) comprising: (a) an extracellular antigen binding domain capable of specifically binding to an epitope of B-cell maturation antigen (BCMA), (b) a transmembrane domain, and (c) an intracellular signaling domain, wherein the subject has received one to three prior lines of therapies, including a therapy with an immunomodulatory drug (IMiD), and is refractory to the IMiD. In some embodiments, the administration of the dose of T cells reduces the risk of disease progression or death in the subject. In some embodiments, the risk of disease progression or death is reduced relative to an administration of daratumumab-pomalidomide-dexamethasone (DPd) or pomalidomide-bortezomib-dexamethasone (PVd) treatment. In some embodiments, the risk of disease progression or death is reduced relative to a standard of care (SOC) therapy comprising administration of either of daratumumab-pomalidomide-dexamethasone (DPd) or pomalidomide-bortezomib-dexamethasone (PVd) treatment. In some embodiments, the risk of disease progression or death is reduced relative to an administration of ide-cel treatment. In some embodiments, the subject has an about 60% to about 75% reduced risk of disease progression or death. In some embodiments, the subject has an about 74% reduced risk of disease progression or death.

In some aspects, provided herein are methods of treating a subject with multiple myeloma, the method comprising administering to the subject a dose of T cells comprising a chimeric antigen receptor (CAR) comprising: (a) an extracellular antigen binding domain capable of specifically binding to an epitope of B-cell maturation antigen (BCMA), (b) a transmembrane domain, and (c) an intracellular signaling domain, wherein the subject has received one to three prior lines of therapies, including a therapy with an immunomodulatory drug (IMiD), and is refractory to the IMiD, and wherein the administration of the dose of T cells is more effective in obtaining a very good partial response (VGPR) or better in the subject as compared to an administration of DPd or PVd treatment. In some embodiments, the VGPR or better after administration of the treatment is about 81.3%. In some embodiments, the VGPR or better after administration of DPd or PVd is about 45.5%. In some embodiments, the VGPR or better after administration of a standard of care (SOC) therapy comprising administration of either of DPd or PVd is about 45.5%. In some embodiments, the treatment is more effective in obtaining a stringent complete response (sCR) in the subject as compared to an administration of DPd or PVd treatment. In some embodiments, the sCR after administration of the treatment is about 58.2%. In some embodiments, the sCR after administration of DPd or PVd is about 15.2%.

Another aspect of the disclosure is a method of treating a subject with multiple myeloma comprising administering to the subject a dose of T cells comprising a chimeric antigen receptor (CAR) comprising: (a) an extracellular antigen binding domain capable of specifically binding to an epitope of B-cell maturation antigen (BCMA), (b) a transmembrane domain, and (c) an intracellular signaling domain, wherein the subject has received one to three prior lines of therapies, including a therapy with an immunomodulatory drug (IMiD), and is refractory to the IMiD.

In some embodiments, the subject has a reduced risk for developing a CAR-T-associated adverse event, wherein optionally the CAR-T-associated adverse event comprises a cytokine release syndrome (CRS) and/or neurotoxicity.

In some embodiments, the CAR-T-associated adverse event is a CRS, further wherein optionally the CRS occurs in the subject at a rate of 60% to about 90%, or a rate of about 76.1%, further wherein optionally maximum toxicity grade of the CRS is Grade 1, Grade 2 or Grade 3.

In some embodiments, the CAR-T-associated adverse event is a CRS, further wherein optionally the CRS occurs in the subject at a rate of about 60% to about 90%, or a rate of about 76.1%, further wherein optionally maximum toxicity grade of the CRS is Grade 1, Grade 2 or Grade 3, further wherein optionally the maximum toxicity grade of the CRS is Grade 1, wherein optionally the maximum toxicity grade of Grade 1 in the subject occurs at a rate of about 52.8%.

In some embodiments, the CAR-T-associated adverse event is a CRS, further wherein optionally the CRS occurs in the subject at a rate of about 60% to about 90%, or a rate of about 76.1%, further wherein optionally maximum toxicity grade of the CRS is Grade 1, Grade 2 or Grade 3, further wherein optionally the maximum toxicity grade of the CRS is Grade 2, wherein optionally the maximum toxicity grade of Grade 2 in the subject occurs at a rate of about 22.2%.

In some embodiments, the CAR-T-associated adverse event is a CRS, further wherein optionally the CRS occurs in the subject at a rate of about 60% to about 90%, or a rate of about 76.1%, further wherein optionally maximum toxicity grade of the CRS is Grade 1, Grade 2 or Grade 3, further wherein optionally the maximum toxicity grade of the CRS is Grade 3, wherein optionally the maximum toxicity grade of Grade 3 in the subject occurs at a rate of about 1.1%;

In some embodiments, the CAR-T-associated adverse event is a CRS, further wherein optionally the CRS occurs in the subject at a rate of about 60% to about 90%, or a rate of about 76.1%, further wherein optionally maximum toxicity grade of the CRS is Grade 1, Grade 2 or Grade 3, further wherein optionally time to first onset of the CRS ranges from about 1 to about 23 days, wherein optionally the time to the first onset of the CRS is at a median of about 8 days.

In one embodiment, the CAR-T-associated adverse event is a CRS, further wherein optionally the CRS occurs in the subject at a rate of about 60% to about 90%, or a rate of about 76.1%, further wherein optionally maximum toxicity grade of the CRS is Grade 1, Grade 2 or Grade 3, further wherein optionally duration of the CRS ranges from about 1 to about 17 days, wherein optionally the duration of the CRS is at a median of about 3 days.

In some embodiments, the CAR-T-associated adverse event is a CRS, further wherein optionally the CRS occurs in the subject at a rate of about 60% to about 90%, or a rate of about 76.1%, further wherein optionally maximum toxicity grade of the CRS is Grade 1, Grade 2 or Grade 3, further wherein optionally the method further comprises administering to the subject tocilizumab, oxygen, a corticosteroid, a vasopressor, or any combination thereof.

In some embodiments, the CAR-T-associated adverse event is neurotoxicity, wherein optionally the neurotoxicity comprises an immune effector cell-associated neurotoxicity syndrome or associated symptom, movement and neurocognitive neurotoxicity, treatment-emergent adverse event of neurotoxicity, a non-immune effector cell-associated neurotoxicity syndrome or associated symptom, or any combination thereof, wherein optionally the neurotoxicity is an immune effector cell-associated neurotoxicity syndrome or associated symptom.

In some embodiments, the CAR-T-associated adverse event is neurotoxicity, wherein optionally the neurotoxicity comprises an immune effector cell-associated neurotoxicity syndrome or associated symptom, movement and neurocognitive neurotoxicity, treatment-emergent adverse event of neurotoxicity, a non-immune effector cell-associated neurotoxicity syndrome or associated symptom, or any combination thereof, wherein optionally the neurotoxicity is an immune effector cell-associated neurotoxicity syndrome or associated symptom, further wherein optionally the immune effector cell-associated neurotoxicity syndrome or associated symptom in the subject occurs at a rate of about 4.5%.

In some embodiments, the CAR-T-associated adverse event is neurotoxicity, wherein optionally the neurotoxicity comprises an immune effector cell-associated neurotoxicity syndrome or associated symptom, movement and neurocognitive neurotoxicity, treatment-emergent adverse event of neurotoxicity, a non-immune effector cell-associated neurotoxicity syndrome or associated symptom, or any combination thereof, wherein optionally the neurotoxicity is an immune effector cell-associated neurotoxicity syndrome or associated symptom, further wherein optionally maximum toxicity grade of the immune effector cell-associated neurotoxicity syndrome or associated symptom is Grade 1 or Grade 2, wherein optionally maximum toxicity grade of the immune effector cell-associated neurotoxicity syndrome or associated symptom is Grade 1, further wherein optionally the maximum toxicity grade of Grade 1 in the subject occurs at a rate of about 3.4%, or wherein optionally maximum toxicity grade of the immune effector cell-associated neurotoxicity syndrome or associated symptom is Grade 2, further wherein optionally the maximum toxicity grade of Grade 2 in the subject occurs at a rate of about 1.1%.

In some embodiments, the CAR-T-associated adverse event is neurotoxicity, wherein optionally the neurotoxicity comprises an immune effector cell-associated neurotoxicity syndrome or associated symptom, movement and neurocognitive neurotoxicity, treatment-emergent adverse event of neurotoxicity, a non-immune effector cell-associated neurotoxicity syndrome or associated symptom, or any combination thereof, wherein optionally the neurotoxicity is an immune effector cell-associated neurotoxicity syndrome or associated symptom, further wherein optionally time to onset of the immune effector cell-associated neurotoxicity syndrome or associated symptom ranges from about 6 to about 15 days, wherein optionally the time to the onset of the immune effector cell-associated neurotoxicity syndrome or associated symptom is at a median of about 9.5 days;

In some embodiments, the CAR-T-associated adverse event is neurotoxicity, wherein optionally the neurotoxicity comprises an immune effector cell-associated neurotoxicity syndrome or associated symptom, movement and neurocognitive neurotoxicity, treatment-emergent adverse event of neurotoxicity, a non-immune effector cell-associated neurotoxicity syndrome or associated symptom, or any combination thereof, wherein optionally the neurotoxicity is an immune effector cell-associated neurotoxicity syndrome or associated symptom, further wherein optionally duration of the immune effector cell-associated neurotoxicity syndrome or associated symptom ranges from about 1 to about 6 days, wherein optionally the duration of the immune effector cell-associated neurotoxicity syndrome or associated symptom is at a median of about 2 days.

In some embodiments, the CAR-T-associated adverse event is neurotoxicity, wherein optionally the neurotoxicity comprises an immune effector cell-associated neurotoxicity syndrome or associated symptom, movement and neurocognitive neurotoxicity, treatment-emergent adverse event of neurotoxicity, a non-immune effector cell-associated neurotoxicity syndrome or associated symptom, or any combination thereof, wherein optionally the neurotoxicity is an immune effector cell-associated neurotoxicity syndrome or associated symptom, further wherein optionally the treatment comprises a corticosteroid and/or tocilizumab.

Another aspect of the disclosure is a method of treating a subject with multiple myeloma comprising administering to the subject a dose of T cells comprising a chimeric antigen receptor (CAR) comprising: (a) an extracellular antigen binding domain capable of specifically binding to an epitope of B-cell maturation antigen (BCMA), (b) a transmembrane domain, and (c) an intracellular signaling domain, wherein the subject has received one to three prior lines of therapies, including a therapy with an immunomodulatory drug (IMiD), and is refractory to the IMiD

In some embodiments, the subject has a reduced risk for developing a CAR-T-associated adverse event, wherein optionally the CAR-T-associated adverse event comprises CAR-T cell neurotoxicity, wherein optionally the CAR-T cell neurotoxicity in the subject occurs at a rate of about 17.0%, wherein optionally the CAR-T cell neurotoxicity comprises Grade 3/4 neurotoxicity, Grade 5 neurotoxicity, cranial nerve palsy, peripheral neuropathy, a movement and neurocognitive treatment-emergent adverse event, or any combination thereof.

In some embodiments, the CAR-T cell neurotoxicity is Grade 3/4 neurotoxicity that occurs in the subject at a rate of about 2.3%.

In some embodiments, the CAR-T cell neurotoxicity is Grade 5 neurotoxicity.

In some embodiments, the CAR-T cell neurotoxicity is cranial nerve palsy, wherein optionally the cranial nerve palsy in the subject occurs at a rate of about 9.1%.

In some embodiments, the CAR-T cell neurotoxicity is cranial nerve palsy, wherein optionally the cranial nerve palsy in the subject occurs at a rate of about 9.1%, the cranial nerve palsy is Grade 2 or Grade 3 cranial nerve palsy, further wherein optionally the cranial nerve palsy is Grade 2 cranial nerve palsy that occurs in the subject at a rate of about 8.0%, and further wherein optionally the cranial nerve palsy is Grade 3 cranial nerve palsy that occurs in the subject at a rate of about 1.1%.

In some embodiments, the CAR-T cell neurotoxicity is cranial nerve palsy, wherein optionally the cranial nerve palsy in the subject occurs at a rate of about 9.1%, time to onset of the cranial nerve palsy after administering the dose of the T cells to the subject ranges from about 17 days to about 60 days, further wherein optionally the time to onset of the cranial nerve palsy after administering the dose of the T cells to the subject is at a median of about 21 days.

In some embodiments, the CAR-T cell neurotoxicity is cranial nerve palsy, wherein optionally the cranial nerve palsy in the subject occurs at a rate of about 9.1% and the cranial nerve palsy affects cranial nerve III, V, or VII.

In some embodiments, the CAR-T cell neurotoxicity is cranial nerve palsy, wherein optionally the cranial nerve palsy in the subject occurs at a rate of about 9.1% and the duration of the cranial nerve palsy ranges from about 15 days to about 262 days, and further wherein optionally duration of the cranial nerve palsy is at a median of about 77 days.

In some embodiments, the CAR-T cell neurotoxicity is cranial nerve palsy, wherein optionally the cranial nerve palsy in the subject occurs at a rate of about 9.1%, and the method further comprises administering the subject a treatment that comprises a corticosteroid.

In some embodiments, the CAR-T cell neurotoxicity is peripheral neuropathy, wherein optionally the peripheral neuropathy in the subject occurs at a rate of about 2.8%. In some embodiments, the peripheral neuropathy is Grade 1. In some embodiments, the Grade 1 peripheral neuropathy occurs at a rate of about 1.1%. In some embodiments, the peripheral neuropathy is Grade 2. In some embodiments, the Grade 2 peripheral neuropathy occurs at a rate of about 1.1%. In some embodiments, the peripheral neuropathy is Grade 3. In some embodiments, the Grade 3 peripheral neuropathy occurs at a rate of about 0.6%.

In some embodiments, the CAR-T cell neurotoxicity is a movement and neurocognitive treatment-emergent adverse event, wherein optionally the movement and neurocognitive treatment-emergent adverse event is Grade 1, further wherein optionally the Grade 1 movement and neurocognitive treatment-emergent adverse event in the subject occurs at a rate of about 0.6%.

Features which are described in the context of separate aspects and embodiments of the disclosure may be used together and/or be interchangeable. Similarly, features described in the context of a single embodiment may also be provided separately or in any suitable sub-combination. All methods described herein, however expressed, may be described as corresponding uses, in particular medical uses.

### BRIEF DESCRIPTION OF THE DRAWINGS

FIG. 1 shows the expression of BCMA antigen on the surface of GC, memory and plasmablast cells in the lymph node, long-lived plasma cells in the bone marrow LN and MALT, and on multiple myeloma cells. BAFF-R antigen is not expressed on plasmablast cells, long-lived plasma cells, or multiple myeloma cells. TACI is expressed on memory and plasmablast cells, long-lived plasma cells, and multiple myeloma cells. CD138 is expressed only on long-lived plasma cells and multiple myeloma cells.

FIG. 2 shows the design of the ciltacabtagene autoleucel CAR. Ciltacabtagene autoleucel comprises two VHH domains, as opposed to a single VL domain and a single VH domain found on various other CARs. Ciltacabtagene autoleucel comprises intracellular CD137 and human CD3 zeta domains.

FIG. 3 shows a schematic for preparing virus encoding ciltacabtagene autoleucel CAR, transduction of the virus into a T cell from the patient, and then preparation of CAR T cells expressing ciltacabtagene autoleucel.

FIG. 4 shows a schematic of study design for ciltacabtagene autoleucel CAR T-cells. The patient population includes those with relapsed or refractory multiple myeloma, with 1 to 3 prior lines of therapies including an immunomodulatory drug or double refractory to PI/IMiD and prior PI, IMiD, and anti-CD38 exposure. A primary objective is comparing efficacy and safety of ciltacabtagene autoleucel CAR T-cells with physician's choice between two highly effective standard-of-care therapies in the above-described patient population in a randomized controlled trial (Phase 3).

FIG. 5 is a graph showing study subject disposition at each treatment arm.

FIGS. 6A-6C show Kaplan-Meier Intent-to-Treat Analysis. FIG. 6A shows progression-free survival by treatment arm. FIG. 6B shows progression-free survival by treatment arm and stratified by number of prior lines of therapy. FIG. 6C shows overall survival by treatment arm.

FIG. 7 shows forest plot of subgroup analysis on progression-free survival. Abbreviations: cilta-cel, ciltacabtagene autoleucel; DPd, daratumumab-pomalidomide-dexamethasone; ECOG, Eastern Cooperative Oncology Group; IMID, immunomodulatory drug; ISS, international staging system; MM, multiple myeloma; NCI, National Cancer Institute; PI, proteasome inhibitor; PVd, pomalidomide-bortezomib-dexamethasone; SOC, standard-of-care. ^{a}Hazard ratio and 95% CI from a Cox proportional hazards model with treatment as the sole explanatory variable, including only PFS events that occurred >8 weeks post-randomization. A hazard ratio <1 indicates an advantage for the cilta-cel arm. ^{b}Based on the interactive web response system randomization strata. ^{c}Based on serum β-2 microglobulin and albumin. ^{d}Based on subjects with measurable disease in serum. ^{e}positive for del(17p), t(14;16), t(4;14), and/or gain/amp(1q) by FISH testing. Protocol-defined high-risk cytogenetics refers to any of 4 markers abnormal. ^{f}Based on the Modification of Diet in Renal Disease (MDRD) formula.

FIGS. 8A-8D show the comparison of PFS and OS responses in patients who received cilta-cel in CARTITUDE-1 and in CARTITUDE-4. FIG. 8A shows PFS in patients who received cilta-cel as study treatment in CARTITUDE-1. FIG. 8B shows PFS in patients who received cilta-cel as study treatment in CARTITUDE-4. FIG. 8C shows OS in patients who received cilta-cel as study treatment in CARTITUDE-1. FIG. 8D shows OS in patients who received cilta-cel as study treatment in CARTITUDE-4.

### DETAILED DESCRIPTION

The disclosure also provides related nucleic acids, recombinant expression vectors, host cells, populations of cells, antibodies, or antigen binding portions thereof, and pharmaceutical compositions relating to the immune cells and CAR-expressing T cells of the disclosure. Dosage regimens and dosage forms, and methods of treatment with the CAR-T cells are also provided.

Several aspects and embodiments of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the disclosure can be practiced without one or more of the specific details or practiced with other methods, protocols, reagents, cell lines and animals. The present disclosure is not limited by the illustrated ordering of acts or events, as some acts may occur in different orders and/or concurrently with other acts or events. Furthermore, not all illustrated acts, steps or events are required to implement a methodology in accordance with the present disclosure.

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The term “antibody” includes monoclonal antibodies (including full length 4-chain antibodies or full length heavy-chain only antibodies which have an immunoglobulin Fc region), antibody compositions with polyepitopic specificity, multispecific antibodies (e.g., bispecific antibodies, diabodies, and single-chain molecules), as well as antibody fragments (e.g., Fab, F(ab′)2, and Fv). The term “immunoglobulin” (Ig) is used interchangeably with “antibody” herein. Antibodies contemplated herein include single-domain antibodies, such as heavy chain only antibodies.

The term “heavy chain-only antibody” or “HCAb” refers to a functional antibody, which comprises heavy chains, but lacks the light chains usually found in 4-chain antibodies. Camelid animals (such as camels, llamas, or alpacas) are known to produce HCAbs.

The term “single-domain antibody” or “sdAb” refers to a single antigen-binding polypeptide having three complementary determining regions (CDRs). The sdAb alone is capable of binding to the antigen without pairing with a corresponding CDR-containing polypeptide. In some cases, single-domain antibodies are engineered from camelid HCAbs, and their heavy chain variable domains are referred herein as “VHHs”. Some VHHs may also be known as “Nanobodies”. A camelid sdAb is one of the smallest known antigen-binding antibody fragments (see, e.g., Hamers-Casterman et al., Nature 363:446-8 (1993); Greenberg et al., Nature 374:168-73 (1995); Hassanzadeh-Ghassabeh et al., Nanomedicine (Lond), 8:1013-26 (2013)). A basic VHH has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites. Heavy-chain only antibodies from the Camelid species have a single heavy chain variable region, which is referred to as “VHH” domain. VHH is thus a special type of variable region.

The term “variable” refers to the fact that certain segments of the variable domains differ extensively in sequence among antibodies. The V domain (i.e., variable domain) mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a j-sheet configuration, connected by three HVRs, which form loops connecting, and in some cases forming part of, the j-sheet structure. The HVRs in each chain are held together in close proximity by the FR regions and contribute to the formation of the antigen binding site of antibodies (with the HVRs from the other chain, if the antibody is not a sdAb or HCAb) (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The terms “fragment of an antibody”, “antibody fragment”, “functional fragment of an antibody”, and “antigen-binding portion” are used interchangeably herein to mean one or more fragments or portions of an antibody that retain the ability to specifically bind to an antigen (see, generally, Holliger et al., Nat. Biotech., 23(9): 1 126-1129 (2005)). The antigen recognition moiety of the CARs encoded by the nucleic acid sequences disclosed herein can contain any BCMA-binding antibody fragment. The antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the VL, VH, CL, and CHI domains; (ii) a F(ab′)2 fragment, which is a bivalent fragment comprising two Fab fragments linked by a disulfide bridge at the hinge region; (iii) a Fv fragment consisting of the VL and VH domains of a single arm of an antibody; (iv) a single chain Fv (scFv), which is a monovalent molecule consisting of the two domains of the Fv fragment (i.e., VL and VH) joined by a synthetic linker which enables the two domains to be synthesized as a single polypeptide chain (see, e.g., Bird et al., Science, 242: 423-426 (1988); Huston et al., Proc. Natl. Acad. Sci. USA, 85: 5879-5883 (1988); and Osbourn et al., Nat. Biotechnol, 16: 778 (1998)) and (v) a diabody, which is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites. Antibody fragments are known in the art and are described in more detail in, e.g., U.S. Patent Application Publication 2009/0093024 A1.

As used herein, the terms “specifically binds”, “specifically recognizes”, or “specific for” refer to measurable and reproducible interactions such as binding between a target and an antigen binding protein (such as a CAR or a VHH), which is determinative of the presence of the target in the presence of a heterogeneous population of molecules including biological molecules.

The term “specificity” refers to selective recognition of an antigen binding protein (such as a CAR or a VHH) for a particular epitope of an antigen. Natural antibodies, for example, are monospecific.

A “chimeric antigen receptor” or “CAR” is an artificially constructed hybrid protein or polypeptide containing the antigen binding domains of an antibody (or antibody fragment) linked to T-cell signaling domains. Characteristics of CARs can include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigens independent of antigen processing, thus bypassing a major mechanism of tumor evasion. Moreover, when expressed in T-cells, advantageously, CARs do not dimerize with endogenous T cell receptor (TCR) α- and β-chains. T cells expressing a CAR are referred to herein as CAR T cells, CAR-T cells or CAR modified T cells, and these terms are used interchangeably herein. The cell can be genetically modified to stably express an antibody binding domain on its surface, conferring novel antigen specificity that is MHC independent. “BCMA CAR” refers to a CAR having an extracellular binding domain specific for BCMA. “Bi-epitope CAR” refers to a CAR having an extracellular binding domain specific for two different epitopes on BCMA.

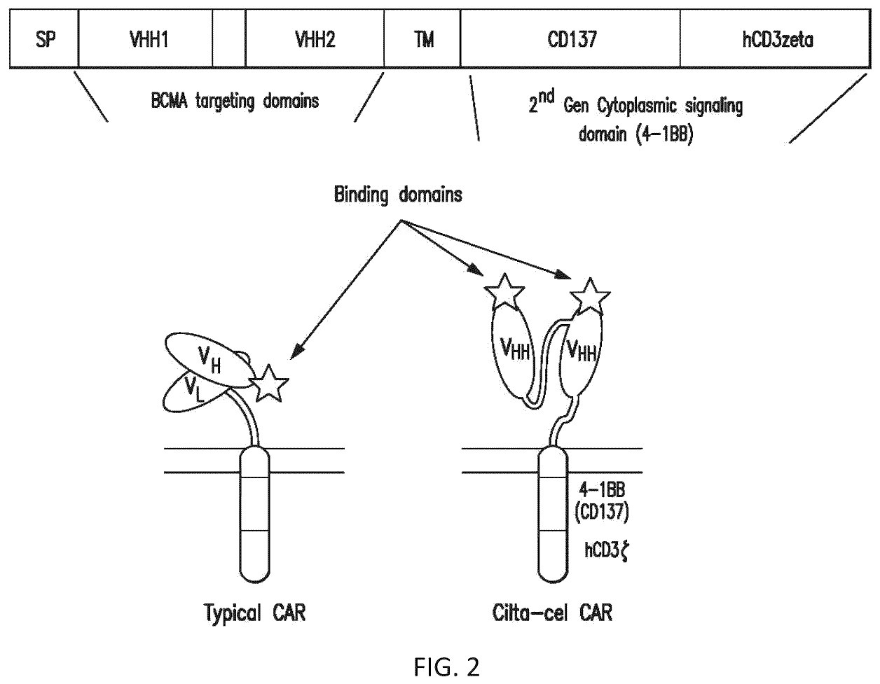

“Ciltacabtagene autoleucel” (“cilta-cel”) is a chimeric antigen receptor T cell (CAR-T) therapy comprising two B-cell maturation antigen (BCMA)-targeting VHH domains designed to confer avidity for BCMA. Cilta-cel can comprise T lymphocytes transduced with the ciltacabtagene autoleucel CAR, a CAR encoded by a lentiviral vector. The CAR targets the human B cell maturation antigen (BCMA CAR). A diagram of the lentiviral vector encoding cilta-cel CAR is provided in FIG. 2. The amino acid sequence of the cilta-cel CAR is the amino acid sequence of SEQ ID NO: 17.

The terms “express” and “expression” mean allowing for or causing the information in a gene or DNA sequence to become produced. For example, expression can take the form of producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular or transmembrane.

The terms “treat” or “treatment” refer to therapeutic treatment wherein the object is to slow down or lessen an undesired physiological change or disease, or provide a beneficial or desired clinical outcome during treatment. Beneficial or desired clinical outcomes include alleviation of symptoms, diminishment of extent of disease, stabilized (i.e., not worsening) state of disease, delay or slowing of disease progression, amelioration or palliation of the disease state, and/or remission (whether partial or total), whether detectable or undetectable. “Treatment” can also mean prolonging survival as compared to expected survival if a subject was not receiving treatment. Those in need of treatment include those subjects already with the undesired physiological change or disease as well as those subjects prone to having the physiological change or disease. Treatment may involve a treatment agent, also referred to herein as a “medicament” or “medication,” that may be intended to help achieve the beneficial or desired clinical outcome of interest by its action. Treatment agents or medicaments may be administered to a subject by many routes, including at least intravenous and oral routes. The term “intravenous,” in connection to the administration of treatment agents or medicaments, refers to the administration of said treatment agents or medicaments within one or more veins. The term “oral,” in connection to the administration of treatment agents or medicaments, refers to the administration of said treatment agents or medicaments via an oral passage such as the mouth.

As used herein, the term “subject” refers to an animal. The terms “subject” and “patient” may be used interchangeably herein in reference to a subject. As such, a “subject” includes a human that is being treated for a disease, or prevention of a disease, as a patient. The methods described herein may be used to treat an animal subject belonging to any classification. Examples of such animals include mammals. Mammals, include, but are not limited to, mammals of the order Rodentia, such as mice and hamsters, and mammals of the order Logomorpha, such as rabbits. The mammals may be of the order Carnivora, including felines (cats) and canines (dogs). The mammals may be of the order Artiodactyla, including bovine (cows) and swine (pigs) or of the order Perssodactyla, including equines (horses). The mammals may be of the order Primates, Ceboids, or Simoids (monkeys) or of the order Anthropoids (humans and apes). In some embodiments, the mammal is a human.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “line of therapy,” as used in connection with methods of treatment herein, refers to one or more cycles of a planned treatment program, which may have consisted of one or more planned cycles of single-agent therapy or combination therapy, as well as a sequence of treatments administered in a planned manner. For example, a planned treatment approach of induction therapy followed by autologous stem cell transplantation followed by maintenance is one line of therapy. A new line of therapy is considered to have started when a planned course of therapy has been modified to include other treatment agents or medicaments (alone or in combination) as a result of disease progression, relapse, or toxicity. A new line of therapy is also considered to have started when a planned period of observation off therapy had been interrupted by a need for additional treatment for the disease.

The term “refractory” as used in connection to treatment with a particular treatment agent or medicament herein, refers to diseases or disease subjects that fail to respond to said treatment agent or medicament. The phrase “refractory myeloma” refers to disease that is nonresponsive while on primary or salvage therapy, or progressed within 60 days of last therapy.

The phrase “nonresponsive disease” refers to either failure to achieve minimal response or development of progressive disease while on therapy.

The phrase “hazard ratio” refers to a measure of the relative rate of progression to an endpoint as compared to a control group. In outcome-based clinical trials, a reduction in the hazard ratio for a test arm as compared to the control indicates the treatment used in the test arm reduces the risk of the endpoint, in the case of the studies described herein, disease progression or death. Preferably hazard ratio is calculated per a stratified constant piecewise weighted log-rank test.

The terminology used herein is for the purpose of describing particular aspects or embodiments only and is not intended to be limiting. As used herein, the indefinite articles “a”, “an” and “the” should be understood to include plural reference unless the context clearly indicates otherwise.

Throughout this disclosure, various aspects and embodiments of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

### Vectors

Polynucleotide sequences encoding the CARs described in the present application can be obtained using standard recombinant techniques. Desired polynucleotide sequences may be isolated and sequenced from antibody producing cells such as hybridoma cells. Alternatively, polynucleotides can be synthesized using nucleotide synthesizers or PCR techniques.

The disclosure also provides a vector comprising the nucleic acid sequence encoding the CARs disclosed herein. The vector can be, for example, a plasmid, a cosmid, a viral vector (e.g., retroviral or adenoviral), or a phage. Suitable vectors and methods of vector preparation are well known in the art (see, e.g., Sambrook et al. and Ausubel et al.).

In addition to the nucleic acid sequences encoding the CARs disclosed herein, the vector preferably comprises expression control sequences, such as promoters, enhancers, polyadenylation signals, transcription terminators, internal ribosome entry sites (IRES), and the like, that provide for the expression of the nucleic acid sequence in a host cell. Exemplary expression control sequences are known in the art and described in, for example, Goeddel, Gene Expression Technology: Methods in Enzymology, Vol. 185, Academic Press, San Diego, Calif. (1990).

In some embodiments, the vector comprises a promoter. A large number of promoters recognized by a variety of potential host cells are well known. The selected promoter can be operably linked to cistron DNA encoding the CARs disclosed herein by removing the promoter from the source DNA via restriction enzyme digestion and inserting the isolated promoter sequence into the vector of the present application. A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, and the RSV promoter. Inducible promoters include, for example, the Tet system (U.S. Pat. Nos. 5,464,758 and 5,814,618), the Ecdysone inducible system (No et al., Proc. Natl. Acad. Sci., 93: 3346-3351 (1996)), the T-REX™ system (Invitrogen, Carlsbad, CA), LACSWITCH™ System (Stratagene, San Diego, CA), and the Cre-ERT tamoxifen inducible recombinase system (Indra et al., Nuc. Acid. Res., 27: 4324-4327 (1999); Nuc. Acid. Res., 28: e99 (2000); U.S. Pat. No. 7,112,715; and Kramer & Fussenegger, Methods Mol. Biol, 308: 123-144 (2005)).

In some embodiments, the vector comprises an “enhancer”. The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (e.g., from depositories such as the ATCC as well as other commercial or individual sources). A number of polynucleotides comprising promoters (such as the commonly used CMV promoter) also comprise enhancer sequences. Enhancers can be located upstream, within, or downstream of coding sequences. The term “Ig enhancers” refers to enhancer elements derived from enhancer regions mapped within the immunoglobulin (Ig) locus. Such Ig enhancers include for example, the heavy chain (mu) 5′ enhancers, light chain (kappa) 5′ enhancers, kappa and mu intronic enhancers, and 3′ enhancers (see generally Paul W. E. (ed), Fundamental Immunology, 3rd Edition, Raven Press, New York (1993), pages 353-363; and U.S. Pat. No. 5,885,827).

In some embodiments, the vector comprises a “selectable marker gene.” The term “selectable marker gene”, as used herein, refers to a nucleic acid sequence that allows cells expressing the nucleic acid sequence to be specifically selected for or against, in the presence of a corresponding selective agent. Suitable selectable marker genes are known in the art and described in, e.g., International Patent Application Publications WO 1992/08796 and WO 1994/28143; Wigler et al., Proc. Natl. Acad. Sci. USA, 77: 3567 (1980); O'Hare et al., Proc. Natl. Acad. Sci. USA, 78: 1527 (1981); Mulligan & Berg, Proc. Natl. Acad. Sci. USA, 78: 2072 (1981); Colberre-Garapin et al., J. Mol. Biol., 150: 1 (1981); Santerre et al., Gene, 30: 147 (1984); Kent et al., Science, 237: 901-903 (1987); Wigler et al., Cell, IP. 223 (1977); Szybalska & Szybalski, Proc. Natl. Acad. Sci. USA, 48: 2026 (1962); Lowy et al., Cell, 22: 817 (1980); and U.S. Pat. Nos. 5,122,464 and 5,770,359.

In some embodiments, the vector is an “episomal expression vector” or “episome,” which is able to replicate in a host cell, and persists as an extrachromosomal segment of DNA within the host cell in the presence of appropriate selective pressure (see, e.g., Conese et al., Gene Therapy, 11: 1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Invitrogen (Carlsbad, CA) and pB-CMV from Stratagene (La Jolla, CA) represent non-limiting examples of an episomal vector that uses T-antigen and the SV40 origin of replication in lieu of EBNAl and oriP.

In some embodiments, the vector is an “integrating expression vector,” which may randomly integrate into the host cell's DNA or may include a recombination site to enable recombination between the expression vector and a specific site in the host cell's chromosomal DNA. Such integrating expression vectors may utilize the endogenous expression control sequences of the host cell's chromosomes to effect expression of the desired protein. Examples of vectors that integrate in a site specific manner include, for example, components of the flp-in system from Invitrogen (Carlsbad, CA) (e.g., pcDNA™5/FRT), or the cre-lox system, such as can be found in the pExchange-6 Core Vectors from Stratagene (La Jolla, CA). Examples of vectors that randomly integrate into host cell chromosomes include, for example, pcDNA3.1 (when introduced in the absence of T-antigen) from Invitrogen (Carlsbad, CA), and pCI or pFNI OA (ACT) FLEXI™ from Promega (Madison, WI).

In some embodiments, the vector is a viral vector. Representative viral expression vectors include, but are not limited to, the adenovirus-based vectors (e.g., the adenovirus-based Per.C6 system available from Crucell, Inc. (Leiden, The Netherlands)), lentivirus-based vectors (e.g., the lentiviral-based pLPl from Life Technologies (Carlsbad, CA)), and retroviral vectors (e.g., the pFB-ERV plus pCFB-EGSH from Stratagene (La Jolla, CA)). In a preferred aspect, the viral vector is a lentivirus vector.

The vector comprising the inventive nucleic acid encoding the CAR can be introduced into a host cell that is capable of expressing the CAR encoded thereby, including any suitable prokaryotic or eukaryotic cell. Preferred host cells are those that can be easily and reliably grown, have reasonably fast growth rates, have well characterized expression systems, and can be transformed or transfected easily and efficiently.

As used herein, the term “host cell” refers to any type of cell that can contain the expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5a E. coli cells, Chinese hamster ovarian cells, monkey VERO cells, COS cells, HEK 293 cells, and the like. In a preferred aspect, the host cells are HEK 293 cells. In some embodiments, the HEK 293 cells are derived from the ATCC SD-3515 line. In some embodiments, the HEK 293 cells are derived from, the IU-VPF MCB line. In some embodiments, the HEK 293 cells are derived from the IU-VPF MWCB line. In some embodiments, the host cell can be a peripheral blood lymphocyte (PBL), a peripheral blood mononuclear cell (PBMC), or a natural killer (NK). Preferably, the host cell is a natural killer (NK) cell. More preferably, the host cell is a T-cell.

For purposes of amplifying or replicating the recombinant expression vector, the host cell may be a prokaryotic cell, e.g., a DH5a cell. For purposes of producing a virus from a viral expression vector, the host cell may be a eukaryotic cell, e.g., a HEK 293 cell. For purposes of producing a recombinant CAR, the host cell can be a mammalian cell. The host cell preferably is a human cell. The host cell can be of any cell type, can originate from any type of tissue, and can be of any developmental stage. Methods for selecting suitable mammalian host cells and methods for transformation, culture, amplification, screening, and purification of cells are known in the art.

In some embodiments, the disclosure provides an isolated host cell which expresses the nucleic acid sequence encoding the CARs described herein.

In some embodiments, the host cell is a T-cell. The T-cell of the disclosure can be any T-cell, such as a cultured T-cell, e.g., a primary T-cell, or a T-cell from a cultured T-cell line, or a T-cell obtained from a mammal. If obtained from a mammal, the T-cell can be obtained from numerous sources, including but not limited to blood, bone marrow, lymph node, the thymus, or other tissues or fluids. T-cells can also be enriched for or purified. The T-cell preferably is a human T-cell (e.g., isolated from a human). The T-cell can be of any developmental stage, including but not limited to, a CD4+/CD8+ double positive T-cell, a CD4+ helper T-cell, e.g., Th, and Th2 cells, a CD8+T− cell (e.g., a cytotoxic T-cell), a tumor infiltrating cell, a memory T-cell, a naive T-cell, and the like. In one aspect, the T-cell is a CD8+ T-cell or a CD4+ T-cell. T-cell lines are available from, e.g., the American Type Culture Collection (ATCC, Manassas, VA), and the German Collection of Microorganisms and Cell Cultures (DSMZ) and include, for example, Jurkat cells (ATCC TIB-152), Sup-Tl cells (ATCC CRL-1942), RPMI 8402 cells (DSMZ ACC-290), Karpas 45 cells (DSMZ ACC-545), and derivatives thereof.

In some embodiments, the host cell is a natural killer (NK) cell. NK cells are a type of cytotoxic lymphocyte that plays a role in the innate immune system. NK cells are defined as large granular lymphocytes and constitute a third kind of cells differentiated from the common lymphoid progenitor which also gives rise to B and T lymphocytes (see, e.g., Immunobiology, 5th ed., Janeway et al., eds., Garland Publishing, New York, NY (2001)). NK cells differentiate and mature in the bone marrow, lymph node, spleen, tonsils, and thymus. Following maturation, NK cells enter into the circulation as large lymphocytes with distinctive cytotoxic granules. NK cells are able to recognize and kill some abnormal cells, such as, for example, some tumor cells and virus-infected cells, and are thought to be important in the innate immune defense against intracellular pathogens. As described above with respect to T-cells, the NK cell can be any NK cell, such as a cultured NK cell, e.g., a primary NK cell, or an NK cell from a cultured NK cell line, or an NK cell obtained from a mammal. If obtained from a mammal, the NK cell can be obtained from numerous sources, including but not limited to blood, bone marrow, lymph node, the thymus, or other tissues or fluids. NK cells can also be enriched for or purified. The NK cell preferably is a human NK cell (e.g., isolated from a human). NK cell lines are available from, e.g., the American Type Culture Collection (ATCC, Manassas, VA) and include, for example, NK-92 cells (ATCC CRL-2407), NK92MI cells (ATCC CRL-2408), and derivatives thereof.

In some embodiments, the nucleic acid sequences encoding a CAR may be introduced into a cell by “transfection”, “transformation”, or “transduction”. “Transfection”, “transformation”, or transduction”, as used herein, refer to the introduction of one or more exogenous polynucleotides into a host cell by using physical or chemical methods.

Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)). Phage or viral vectors can be introduced into host cells, after growth of infectious particles in suitable packaging cells, many of which are commercially available.

### Chimeric Antigen Receptors

International Patent Publication No. WO 2018/028647 is incorporated by reference herein in its entirety. US Patent Publication No. 2018/0230225 is incorporated by reference herein in its entirety. Both publications describe chimeric antigen receptors (CARs) targeting BCMA useful in the present disclosure.

The disclosure provides for methods of treating a subject with cells expressing a chimeric antigen receptor (CAR). The CAR comprises an extracellular antigen binding domain comprising one or more single-domain antibodies. In various aspects and embodiments, there is provided a CAR targeting BCMA (also referred herein as “BCMA CAR”) comprising a polypeptide comprising: (a) an extracellular antigen binding domain comprising an anti-BCMA binding moiety; (b) a transmembrane domain; and (c) an intracellular signaling domain. In some embodiments, the anti-BCMA binding moiety is camelid, chimeric, human, or humanized. In some embodiments, the intracellular signaling domain comprises a primary intracellular signaling domain of an immune effector cell (such as T cell). In some embodiments, the primary intracellular signaling domain is derived from CD4. In some embodiments, the primary intracellular signaling domain is derived from CD3-zeta. In some embodiments, the intracellular signaling domain comprises a co-stimulatory signaling domain. In some embodiments, the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 and combinations thereof. In certain embodiments, the transmembrane domain is derived from CD137.

In some embodiments, the BCMA CAR further comprises a hinge domain (such as a CD8-alpha hinge domain) located between the C-terminus of the extracellular antigen binding domain and the N-terminus of the transmembrane domain. In some embodiments, the BCMA CAR further comprises a signal peptide (such as a CD8-alpha signal peptide) located at the N-terminus of the polypeptide. In some embodiments, the polypeptide comprises from the N-terminus to the C-terminus: a CD8-alpha signal peptide, the extracellular antigen-binding domain, a CD8-alpha hinge domain, a CD28 transmembrane domain, a first co-stimulatory signaling domain derived from CD28, a second co-stimulatory signaling domain derived from CD137, and a primary intracellular signaling domain derived from CD4. In some embodiments, the polypeptide comprises from the N-terminus to the C-terminus: a CD8-alpha signal peptide, the extracellular antigen-binding domain, a CD8-alpha hinge domain, a CD8-alpha transmembrane domain, a second co-stimulatory signaling domain derived from CD137, and a primary intracellular signaling domain derived from CD3-zeta. In some embodiments, the BCMA CAR is monospecific. In some embodiments, the BCMA CAR is monovalent.

The present application also provides CARs that have two or more (including, but not limited to, any one of 2, 3, 4, 5, 6, or more) binding moieties that specifically bind to an antigen, such as BCMA. In some embodiments, one or more of the binding moieties are antigen binding fragments. In some embodiments, one or more of the binding moieties comprise single-domain antibodies. In some embodiments, one or more of the binding moieties comprise a VHH.

In some embodiments, the CAR is a multivalent (such as bivalent, trivalent, or of higher number of valencies) CAR comprising a polypeptide comprising: (a) an extracellular antigen binding domain comprising a plurality (such as at least about any one of 2, 3, 4, 5, 6, or more) of binding moieties specifically binding to an antigen (such as a tumor antigen); (b) a transmembrane domain; and (c) an intracellular signaling domain.

In some embodiments, the binding moieties, such as VHHs (including the plurality of VHHs, or the first VHH and/or the second VHH) are camelid, chimeric, human, or humanized. In some embodiments, the binding moieties or VHHs are connected to each other via peptide bonds or peptide linkers. In some embodiments, each peptide linker is no more than about 50 (such as no more than about any one of 35, 25, 20, 15, 10, or 5) amino acids long.

In some embodiments, the first BCMA binding moiety and/or the second BCMA binding moiety is an anti-BCMA VHH. In some embodiments, the first BCMA binding moiety is a first anti-BCMA VHH and the second BCMA binding moiety is a second anti-BCMA VHH.

In some embodiments, the first BCMA binding moiety and the second BCMA binding moiety are connected to each other via a peptide linker. In some embodiments, the peptide linker comprises the amino acid sequence of SEQ ID NO: 3. In some embodiments, the peptide linker comprises a polypeptide encoded by the nucleic acid sequence of SEQ ID NO: 11.

In some embodiments, the CAR further comprises a hinge domain (such as a CD8-alpha hinge domain) located between the C-terminus of the extracellular antigen binding domain and the N-terminus of the transmembrane domain. In some embodiments, the CAR further comprises a signal peptide (such as a CD8-alpha signal peptide) located at the N-terminus of the polypeptide.

Without wishing to be bound by theory, the CARs that are multivalent, or those CARs comprising an extracellular antigen binding domain comprising a first BCMA binding moiety and a second BCMA binding moiety, may be specially suitable for targeting multimeric antigens via synergistic binding by the different antigen binding sites, or for enhancing binding affinity or avidity to the antigen. Improved avidity may allow for a substantial reduction in the dose of CAR-T cells needed to achieve a therapeutic effect, such as a dose ranging from 4.0×10⁴ to 1.0×10⁶ CAR-T cells per kilogram of the mass of the subject, or 3.0×10⁶ to 1.0×10⁸ total CAR-T expressing cells. Monovalent CARs, such as bb2121, may need to be dosed at 5 to 10 times these amounts to achieve a comparable effect. In various embodiments, reduced dosage ranges may provide for substantial reduction in cytokine release syndrome (CRS) and other potentially dangerous side-effects of CAR-T therapy.

The various binding moieties (e.g., an extracellular antigen binding domain comprising a first BCMA binding moiety and a second BCMA binding moiety) in the CARs described herein may be connected to each other via peptide linkers. The peptide linkers connecting different binding moieties (such as VHHs) may be the same or different. Different domains of the CARs may also be connected to each other via peptide linkers. In some embodiments, the binding moieties (such as VHHs) are directly connected to each other without any peptide linkers.

The peptide linker in the CARs described herein can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, 75, 100 or more amino acids long. In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acids to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, about 50 amino acids to about 100 amino acids, or about 1 amino acid to about 100 amino acids.

The CARs of the present application comprise a transmembrane domain that can be directly or indirectly connected to the extracellular antigen binding domain.

The CAR may comprise a T-cell activation moiety. The T-cell activation moiety can be any suitable moiety derived or obtained from any suitable molecule. In one aspect, for example, the T-cell activation moiety comprises a transmembrane domain. The transmembrane domain can be any transmembrane domain derived or obtained from any molecule known in the art. For example, the transmembrane domain can be obtained or derived from a CD8a molecule or a CD28 molecule. Without wishing to be bound by theory, CD8 is a transmembrane glycoprotein that serves as a co-receptor for the T-cell receptor (TCR), and is expressed primarily on the surface of cytotoxic T-cells. The most common form of CD8 exists as a dimer composed of a CD8 alpha (CD8α) and CD8 beta (CD8β) chain. CD28 is expressed on T-cells and provides co-stimulatory signals required for T-cell activation. CD28 is the receptor for CD80 (B7.1) and CD86 (B7.2). In a preferred aspect, the CD8a and CD28 are human.

In addition to the transmembrane domain, the T-cell activation moiety may further comprise an intracellular (i.e., cytoplasmic) T-cell signaling domain. The intercellular T-cell signaling domain can be obtained or derived from a CD28 molecule, a CD3 zeta (ζ) molecule or modified versions thereof, a human Fc receptor gamma (FcRγ) chain, a CD27 molecule, an OX40 molecule, a 4-1BB molecule, or other intracellular signaling molecules known in the art. Without wishing to be bound by theory: (1) CD28 is a T-cell marker important in T-cell co-stimulation; (2) CD3ζ associates with TCRs to produce a signal and contains immunoreceptor tyrosine-based activation motifs (ITAMs); and (3) 4-1BB, also known as CD137, transmits a potent costimulatory signal to T-cells, promoting differentiation and enhancing long-term survival of T lymphocytes. In a preferred aspect, the CD28, CD3 zeta, 4-1BB, OX40, and CD27 are human.

The T-cell activation domain of a CAR encoded by the nucleic acid sequences disclosed herein can comprise any one of aforementioned transmembrane domains and any one or more of the aforementioned intercellular T-cell signaling domains in any combination. For example, the nucleic acid sequences disclosed herein can encode a CAR comprising a CD28 transmembrane domain and intracellular T-cell signaling domains of CD28 and CD3 zeta. Alternatively, for example, the nucleic acid sequences disclosed herein can encode a CAR comprising a CD8a transmembrane domain and intracellular T-cell signaling domains of CD28, CD3 zeta, the Fc receptor gamma (FcRγ) chain, and/or 4-1BB.

In some embodiments, the CAR polypeptide further comprises a signal peptide located at the N-terminus of the polypeptide. In some embodiments, the signal peptide is derived from CD8-alpha. In some embodiments, the signal peptide comprises the amino acid sequence of SEQ ID NO: 1. In some embodiments, signal peptide comprises a polypeptide encoded by the nucleic acid sequence of SEQ ID NO: 9.

In certain embodiments, the transmembrane domain comprises the amino acid sequence of SEQ ID NO: 6. In certain embodiments, the transmembrane domain comprises a polypeptide encoded by the nucleic acid sequence of SEQ ID NO: 14.

In some embodiments, the intracellular signaling domain comprises a primary intracellular signaling domain of an immune effector cell. In some embodiments, the intracellular signaling domain is derived from CD3. In some embodiments, the intracellular signaling domain comprises at least one co-stimulatory signaling domain. In some embodiments, the intracellular signaling domain comprises the amino acid sequence of SEQ ID NO: 8. In some embodiments, the intracellular signaling domain comprises a polypeptide encoded by the nucleic acid sequence of SEQ ID NO: 16. In some embodiments, the intracellular signaling domain comprises the amino acid sequence of SEQ ID NO: 7. In some embodiments, the intracellular signaling domain comprises a polypeptide encoded by the nucleic acid sequence of SEQ ID NO: 15.

In some embodiments, the CAR polypeptide further comprises a hinge domain located between the C-terminus of the extracellular antigen binding domain and the N-terminus of the transmembrane domain. In some embodiments, the hinge domain comprises the amino acid sequence of SEQ ID NO: 5. In some embodiments, the hinge domain comprises a polypeptide encoded by the nucleic acid sequence of SEQ ID NO: 13.

In some embodiments, the CAR comprises one or more of, or all of, SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8. In one aspect, the CAR comprises SEQ ID NO: 17. In some embodiments, the CAR comprises a polypeptide encoded by the nucleic acid sequence of one or more of, or all of, SEQ ID NO: 9, SEQ ID NO: 10, SEQ ID NO: 11, SEQ ID NO: 12, SEQ ID NO: 13, SEQ ID NO: 14, SEQ ID NO: 15 and SEQ ID NO: 16.

In preferred embodiments, the CAR comprises a first VHH domain comprising a CDR1, a CDR2 and a CDR3 of the VHH domain comprising the amino acid sequence of SEQ ID NO: 2, and a second VHH domain comprising a CDR1, a CDR2 and a CDR3 of the VHH domain comprising the amino acid sequence of SEQ ID NO: 4. In preferred embodiments, the first VHH domain is linked to the second VHH domain via a linker comprising the amino acid sequence of SEQ ID NO: 3. In particularly preferred embodiments, the first VHH domain comprises a CDR1 comprising the amino acid sequence of SEQ ID NO: 18, a CDR2 comprising the amino acid sequence of SEQ ID NO: 19, a CDR3 comprising the amino acid sequence of SEQ ID NO: 20, and the second VHH domain comprises a CDR1 comprising the amino acid sequence of SEQ ID NO: 21, a CDR2 comprising the amino acid sequence of SEQ ID NO: 22, and a CDR3 comprising the amino acid sequence of SEQ ID NO: 23. In further preferred embodiments, the CAR comprises a first VHH domain comprising the amino acid sequence of SEQ ID NO: 2, and a second VHH domain comprising the amino acid sequence of SEQ ID NO: 4.

### Immune Effector Cell Compositions

“Immune effector cells” are immune cells that can perform immune effector functions. In some embodiments, the immune effector cells express at least FcγRIII and perform ADCC effector function. Examples of immune effector cells which mediate ADCC include peripheral blood mononuclear cells (PBMC), natural killer (NK) cells, monocytes, cytotoxic T cells, neutrophils, and eosinophils. In some embodiments, the immune effector cells are T cells. In some embodiments, the T cells are autologous T cells. In some embodiments, the T cells are allogeneic T cells. In some embodiments, the T cells are CD4+/CD8−, CD4−/CD8+, CD4+/CD8+, CD4−/CD8−, or combinations thereof. In some embodiments, the T cells produce IL-2, TFN, and/or TNF upon expressing the CAR and binding to the target cells, such as CD20+ or CD19+ tumor cells. In some embodiments, the CD8+ T cells lyse antigen-specific target cells upon expressing the CAR and binding to the target cells.

Biological methods for introducing the vector into an immune effector cell include the use of DNA and RNA vectors. Viral vectors have become the most widely used method for inserting genes into mammalian, e.g., human cells. Chemical means for introducing the vector into an immune effector cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle).

Provided herein are dosage forms comprising 3.0×10⁷ to 1.0×10⁸ CAR-T cells comprising a CAR comprising a polypeptide comprising: (a) an extracellular antigen binding domain comprising a first BCMA binding moiety specifically binding to a first epitope of BCMA, and a second BCMA binding moiety specifically binding to a second epitope of BCMA; (b) a transmembrane domain; and (c) an intracellular signaling domain, wherein the first epitope and the second epitope are different. In certain embodiments, the dosage form comprises 3.0×10⁷ to 4.0×10⁷ of the CAR-T cells. In certain embodiments, the dosage form comprises 3.5×10⁷ to 4.5×10⁷ of the CAR-T cells. In certain embodiments, the dosage form comprises 4.0×10⁷ to 5.0×10⁷ of the CAR-T cells. In certain embodiments, the dosage form comprises 4.5×10⁷ to 5.5×10⁷ of the CAR-T cells. In certain embodiments, the dosage form comprises 5.0×10⁷ to 6.0×10⁷ of the CAR-T cells. In certain embodiments, the dosage form comprises 5.5×10⁷ to 6.5×10⁷ of the CAR-T cells. In certain embodiments, the dosage form comprises 6.0×10⁷ to 7.0×10⁷ of the CAR-T cells. In certain embodiments, the dosage form comprises 6.5×10⁷ to 7.5×10⁷ of the CAR-T cells. In certain embodiments, the dosage form comprises 7.0×10⁷ to 8.0×10⁷ of the CAR-T cells. In certain embodiments, the dosage form comprises 7.5×10⁷ to 8.5×10⁷ of the CAR-T cells. In certain embodiments, the dosage form comprises 8.0×10⁷ to 9.0×10⁷ of the CAR-T cells. In certain embodiments, the dosage form comprises 8.5×10⁷ to 9.5×10⁷ of the CAR-T cells. In certain embodiments, the dosage form comprises 9.0×10⁷ to 1.0×10⁸ of the CAR-T cells.

In some embodiments, there are provided dosage forms comprising 3.0×10⁷ to 1.0×10⁸ engineered immune effector cells (such as T-cells) comprising a CAR comprising a polypeptide comprising: (a) an extracellular antigen binding domain comprising a first anti-BCMA VHH specifically binding to a first epitope of BCMA, and a second anti-BCMA VHH specifically binding to a second epitope of BCMA; (b) a transmembrane domain; and (c) an intracellular signaling domain, wherein the first epitope and the second epitope are different. In certain embodiments, the dosage form comprises 3.0×10⁷ to 4.0×10⁷ of the CAR-T cells. In certain embodiments, the dosage form comprises 3.5×10⁷ to 4.5×10⁷ of the CAR-T cells. In certain embodiments, the dosage form comprises 4.0×10⁷ to 5.0×10⁷ of the CAR-T cells. In certain embodiments, the dosage form comprises 4.5×10⁷ to 5.5×10⁷ of the CAR-T cells. In certain embodiments, the dosage form comprises 5.0×10⁷ to 6.0×10⁷ of the CAR-T cells. In certain embodiments, the dosage form comprises 5.5×10⁷ to 6.5×10⁷ of the CAR-T cells. In certain embodiments, the dosage form comprises 6.0×10⁷ to 7.0×10⁷ of the CAR-T cells. In certain embodiments, the dosage form comprises 6.5×10⁷ to 7.5×10⁷ of the CAR-T cells. In certain embodiments, the dosage form comprises 7.0×10⁷ to 8.0×10⁷ of the CAR-T cells. In certain embodiments, the dosage form comprises 7.5×10⁷ to 8.5×10⁷ of the CAR-T cells. In certain embodiments, the dosage form comprises 8.0×10⁷ to 9.0×10⁷ of the CAR-T cells. In certain embodiments, the dosage form comprises 8.5×10⁷ to 9.5×10⁷ of the CAR-T cells. In certain embodiments, the dosage form comprises 9.0×10⁷ to 1.0×10⁸ of the CAR-T cells.

In some embodiments, the cell population of the CAR-T dosage forms described herein comprise a T cell or population of T cells, e.g., at various stages of differentiation. Stages of T cell differentiation include naïve T cells, stem central memory T cells, central memory T cells, effector memory T cells, and terminal effector T cells, from least to most differentiated. After antigen exposure, naïve T cells proliferate and differentiate into memory T cells, e.g., stem central memory T cells and central memory T cells, which then differentiate into effector memory T cells. Upon receiving appropriate T cell receptor, costimulatory, and inflammatory signals, memory T cells further differentiate into terminal effector T cells. See, e.g., Restifo. Blood. 124.4(2014):476-77; and Joshi et al. J. Immunol. 180.3(2008):1309-15.

Naïve T cells can have the following expression pattern of cell surface markers: CCR7+, CD62L+, CD45RO−, CD95−. Stem central memory T cells (Tscm) can have the following expression pattern of cell surface markers: CCR7+, CD62L+, CD45RO−, CD95+. Central memory T cells (Tcm) can have the following expression pattern of cell surface markers: CCR7+, CD62L+, CD45RO+, CD95+. Effector memory T cells (Tem) can have the following expression pattern of cell surface markers: CCR7−, CD62L−, CD45RO+, CD95+. Terminal effector T cells (Teff) can have the following expression pattern of cell surface markers: CCR7−, CD62L−, CD45RO−, CD95+. See, e.g., Gattinoni et al. Nat. Med. 17(2011):1290-7; and Flynn et al. Clin. Translat. Immunol. 3(2014):e20.

### Pharmaceutical Compositions and Formulations

Further provided by the present application are pharmaceutical compositions comprising any one of the anti-BCMA antibodies of the disclosure, or any one of the engineered immune effector cells comprising any one of the CARs (such as BCMA CARs) as described herein, and a pharmaceutically acceptable carrier. Pharmaceutical compositions can be prepared by mixing any of the immune effector cells described herein, having the desired degree of purity, with optional pharmaceutically acceptable carriers, excipients or stabilizers (Remington's Pharmaceutical Sciences 16th edition, Osol, A. Ed. (1980)), in the form of lyophilized formulations or aqueous solutions. In certain embodiments, a pharmaceutical composition of CAR-T cells further comprises an excipient selected from dimethyl sulfoxide (DMSO) or dextran-40. In some embodiments, the formulation provided herein comprises 5% DMSO.

The compositions described herein may be administered as part of a pharmaceutical composition comprising one or more carriers. The choice of carrier will be determined in part by the particular nucleic acid sequence, vector, or host cells expressing the CARs disclosed herein, as well as by the particular method used to administer the nucleic acid sequence, vector, or host cells expressing the CARs disclosed herein. Accordingly, there are a variety of suitable formulations of the pharmaceutical compositions of the disclosure.

For example, the pharmaceutical compositions can contain preservatives. Suitable preservatives may include, for example, methylparaben, propylparaben, sodium benzoate, and benzalkonium chloride. A mixture of two or more preservatives optionally may be used. The preservative or mixtures thereof are typically present in an amount of about 0.0001% to about 2% by weight of the total composition.

In addition, buffering agents may be used in the compositions. Suitable buffering agents include, for example, citric acid, sodium citrate, phosphoric acid, potassium phosphate, and various other acids and salts. A mixture of two or more buffering agents optionally may be used. The buffering agent or mixtures thereof are typically present in an amount of about 0.001% to about 4% by weight of the total composition.

The compositions comprising the nucleic acid sequence encoding the CARs disclosed herein, or host cells expressing the CARs disclosed herein, can be formulated as an inclusion complex, such as cyclodextrin inclusion complex, or as a liposome. Liposomes can serve to target the host cells (e.g., T-cells or NK cells) or the nucleic acid sequences disclosed herein to a particular tissue. Liposomes also can be used to increase the half-life of the nucleic acid sequences disclosed herein. Many methods are available for preparing liposomes, such as those described in, for example, Szoka et al., Ann. Rev. Biophys. Bioeng., 9: 467 (1980), and U.S. Pat. Nos. 4,235,871; 4,501,728; 4,837,028; and 5,019,369. The compositions can employ time-released, delayed release, and sustained release delivery systems such that the delivery of the compositions disclosed herein occurs prior to, and with sufficient time to cause, sensitization of the site to be treated. Many types of release delivery systems are available and known to those of ordinary skill in the art. Such systems can avoid repeated administrations of the composition, thereby increasing convenience to the subject and the physician, and may be particularly suitable for certain composition aspects and embodiments of the disclosure.

In certain embodiments, the CAR-T cells are formulated at a dose of about 1.0×10⁵ to 2.0×10⁵ cells/kg, 1.5×10⁵ to 2.5×10⁵ cells/kg, 2.0×10⁵ to 3.0×10⁵ cells/kg, 2.5×10⁵ to 3.5×10⁵ cells/kg, 3.0×10⁵ to 4.0×10⁵ cells/kg, 3.5×10⁵ to 4.5×10⁵ cells/kg, 4.0×10⁵ to 5.0×10⁵ cells/kg, 4.5×10⁵ to 5.5×10⁵ cells/kg, 5.0×10⁵ to 6.0×10⁵ cells/kg, 5.5×10⁵ to 6.5×10⁵ cells/kg, 6.0×10⁵ to 7.0×10⁵ cells/kg, 6.5×10⁵ to 7.5×10⁵ cells/kg, 7.0×10⁵ to 8.0×10⁵ cells/kg, 7.5×10⁵ to 8.5×10⁵ cells/kg, 8.0×10⁵ to 9.0×10⁵ cells/kg, 8.5×10⁵ to 9.5×10⁵ cells/kg, 9.0×10⁵ to 1.0×10⁶ cells/kg. In a preferred aspect, the dose is formulated at approximately 0.75×10⁶ cells/kg. In certain embodiments, the CAR-T cells are formulated at a dose of less than 1.0×10⁸ cells per subject. Preferably, the dose is administered as a single infusion.

### Methods of Treatment and Uses

The present application further relates to methods and compositions for use in cell immunotherapy.

In some aspects, provided herein is a method for treating cancer in a subject with the compositions provided herein, who has multiple myeloma, has received 1 to 3 prior lines of therapy including a therapy with an immunomodulatory drug (IMiD), and is refractory to the IMiD. In some aspects, provided herein is a composition for use in treating cancer in a subject, who has multiple myeloma, has received 1 to 3 prior lines of therapy including a therapy with an immunomodulatory drug (IMiD), and is refractory to the IMiD. In some embodiments, the subject has received one prior line of therapy. In some embodiments, the subject has received two prior lines of therapy. In some embodiments, the subject has received three prior lines of therapy.

In preferred embodiments, the subject has received prior treatment with an IMiD as part of one or more of the 1 to 3 prior lines of therapy. In some embodiments, the IMiD is lenalidomide. In preferred embodiments, the patient is lenalidomide-refractory. In some embodiments, the prior treatment comprises pomalidomide. In some embodiments the IMiD prior line of therapy comprises a combination of lenalidomide and pomalidomide. In some embodiments, the subject has received prior treatment with a proteasome inhibitor as part of one or more of the 1 to 3 prior lines of therapy. In some embodiments, the proteasome inhibitor is bortezomib, carfilzomib, ixazomib, or any combination thereof. In some embodiments, the subject has received prior treatment with an anti-CD38 antibody as part of one or more of the 1 to 3 prior lines of therapy. In some embodiments, the anti-CD38 antibody is daratumumab and/or isatuximab. In some embodiments, the prior treatment comprises an IMiD (e.g. lenalidomide), a proteasome inhibitor and an anti-CD38 antibody (i.e. 3 prior lines of therapy). In some embodiments, the subject has received 1 prior line of therapy including lenalidomide and is lenalidomide-refractory, and optionally has received one or two further lines of therapy. In some embodiments, the subject has received at least one prior lines of therapy including lenalidomide and a proteasome inhibitor, and optionally has received one or two further lines of therapy.

The therapy can optionally be used to treat the subject that has a high-risk feature, including, for example, a cytogenetic abnormality, International Staging System (ISS) stage III, and/or soft tissue plasmacytomas. In some embodiments, the high-risk feature is a cytogenetic abnormality. In some embodiments, the cytogenetic abnormality is a high-risk cytogenetic abnormality. In some embodiments, the subject has one or more high-risk cytogenetic abnormality selected from a group comprising Gain/amp(1q), del(17p), t(4;14), t(14;16), or any combination thereof. In some embodiments, the cytogenetic abnormality comprises Gain/amp(1q). In some embodiments, the cytogenetic abnormality comprises del(17p). In some aspects, the cytogenetic abnormality comprises t(4;14). In some embodiments, the cytogenetic abnormality comprises t(14;16). t(4;14) and t(14;16) are translocations, where parts of the chromosome are swapped. del(17p) is a loss of part of the short arm of chromosome 17. Gain/amp(1q) indicates gain (e.g., 3 total copies) or amplification (e.g., >3 total copies) of part of the long arm of chromosome 1. In some embodiments, the subject has at least two cytogenetic abnormalities. In other embodiments, the subject has two, three, four, five, or more cytogenetic abnormalities. In other embodiments, the cytogenetic abnormality is a standard-risk cytogenetic abnormality. In some embodiments, the high-risk feature is International Staging System (ISS) stage III. In some embodiments, the high-risk feature is soft tissue plasmacytomas.

In other aspects, the method provided herein comprises first determining whether the subject has a high-risk feature, the high-risk feature being a cytogenetic abnormality, International Staging System (ISS) stage III, and/or soft tissue plasmacytomas; and then administering to the subject who is determined to have the high-risk feature with the compositions provided herein. In some aspects, the subject has multiple myeloma, has received 1 to 3 prior lines of therapy including a therapy with an immunomodulatory drug (IMiD), and is refractory to the IMiD. In some embodiments, the IMiD is lenalidomide. In some embodiments, the high-risk feature is a cytogenetic abnormality. In some embodiments, the cytogenetic abnormality is a high-risk cytogenetic abnormality. In some embodiments, the subject has one or more high-risk cytogenetic abnormality selected from a group comprising Gain/amp(1q), del(17p), t(4;14), t(14;16), or any combination thereof. In some embodiments, the cytogenetic abnormality comprises Gain/amp(1q). In some embodiments, the cytogenetic abnormality comprises del(17p). In some embodiments, the cytogenetic abnormality comprises t(4;14). In some embodiments, the cytogenetic abnormality comprises t(14;16). In some embodiments, the subject has at least two cytogenetic abnormalities. In other embodiments, the subject has two, three, four, five, or more cytogenetic abnormalities. In other embodiments, the cytogenetic abnormality is a standard-risk cytogenetic abnormality. In some embodiments, the high-risk feature is International Staging System (ISS) stage III. In some embodiments, the high-risk feature is soft tissue plasmacytomas. In some embodiments, the subject has received one prior line of therapy. In some embodiments, the subject has received two prior lines of therapies. In some embodiments, the subject has received three prior lines of therapies. In some embodiments, the prior treatment comprises pomalidomide. In some aspects, the prior treatment further comprises a proteasome inhibitor, wherein optionally the proteasome inhibitor is bortezomib, carfilzomib, ixazomib, or any combination thereof. In some embodiments, the prior treatment further comprises an anti-CD38 antibody, and wherein optionally the anti-CD38 antibody is daratumumab and/or isatuximab. In some embodiments, the prior treatment comprises an IMiD (e.g. lenalidomide), a proteasome inhibitor and an anti-CD38 antibody.

In yet other aspects, provided herein is a method of selectively treating a subject with the compositions provided herein, comprising administering to the subject who has been determined to have a high-risk feature such as a cytogenetic abnormality, International Staging System (ISS) stage III, and/or soft tissue plasmacytomas. In some embodiments, the composition provided herein is for use in treating a subject who has been determined to have a high-risk feature such as a cytogenetic abnormality, International Staging System (ISS) stage III, and/or soft tissue plasmacytomas. In some embodiments, the subject has multiple myeloma, has received 1 to 3 prior lines of therapy including a therapy with an immunomodulatory drug (IMiD), and is refractory to the IMiD. In some embodiments, the IMiD is lenalidomide. In some embodiments, the high-risk feature is a cytogenetic abnormality. In some embodiments, the cytogenetic abnormality is a high-risk cytogenetic abnormality. In some embodiments, the subject has one or more high-risk cytogenetic abnormality selected from a group comprising Gain/amp(1q), del(17p), t(4;14), t(14;16), or any combination thereof. In some embodiments, the cytogenetic abnormality comprises Gain/amp(1q). In some embodiments, the cytogenetic abnormality comprises del(17p). In some embodiments, the cytogenetic abnormality comprises t(4;14). In some embodiments, the cytogenetic abnormality comprises t(14;16). In some embodiments, the subject has at least two cytogenetic abnormalities. In other embodiments, the subject has two, three, four, five, or more cytogenetic abnormalities. In other embodiments, the cytogenetic abnormality is a standard-risk cytogenetic abnormality. In some embodiments, the high-risk feature is International Staging System (ISS) stage III. In some embodiments, the high-risk feature is soft tissue plasmacytomas. In some embodiments, the subject has received one prior line of therapy. In some embodiments, the subject has received two prior lines of therapies. In some embodiments, the subject has received three prior lines of therapies. In some embodiments, the prior treatment comprises pomalidomide. In some embodiments, the prior treatment further comprises a proteasome inhibitor, wherein optionally the proteasome inhibitor is bortezomib, carfilzomib, ixazomib, or any combination thereof. In some embodiments, the prior treatment further comprises an anti-CD38 antibody, and wherein optionally the anti-CD38 antibody is daratumumab and/or isatuximab. In some embodiments, the prior treatment comprises an IMiD (e.g. lenalidomide), a proteasome inhibitor and an anti-CD38 antibody.

In some embodiments of the various methods or uses provided herein, the subject has further received a bridging therapy, and the bridging therapy can optionally be of the physician's choice. The bridging therapy can include pomalidomide, bortezomib, dexamethasone, daratumumab, or any combination thereof. It can include pomalidomide, bortezomib and dexamethasone. Another exemplary bridging therapy comprises daratumumab, pomalidomide and dexamethasone. In some embodiments, the subject has received the bridging therapy from about every 20 days to about every 30 days, for example, about every 21 days, or about every 28 days. In some embodiments, the subject has received at least one, two, three, four, or more bridging therapies.

In certain embodiments, the bridging therapy comprises a 28-day cycle comprising daratumumab on days 1, 8, 15, and 22, pomalidomide for 21 days and dexamethasone on days 1, 8, 15, and 22. For example, the bridging therapy may comprise 1800 mg daratumumab on days 1, 8, 15, and 22; 4 mg/day pomalidomide for 21 days, and 40 mg dexamethasone on days 1, 8, 15, and 22 In said embodiments, the daratumumab may be subcutaneous, the pomalidomide may be oral, and the dexamethasone may be oral or intravenous.

In other embodiments, the bridging therapy comprises a 21-day cycle comprising bortezomib on days 1, 4, 8, and 11, pomalidomide for 14 days and dexamethasone on days 1, 2, 4, 5, 8, 9, 11, and 12. For example, the bridging therapy may comprise 1.3 mg/m² bortezomib on days 1, 4, 8, and 11; 4 mg/day pomalidomide for 14 days, and 20 mg dexamethasone on bridging days 1, 2, 4, 5, 8, 9, 11, and 12. In said embodiments, the bortezomib may be subcutaneous, the pomalidomide may be oral, and the dexamethasone may be oral.

In some embodiments, the subject has further received a lymphodepletion therapy, for example following a bridging therapy disclosed herein. In some embodiments, the lymphodepletion therapy comprises cyclophosphamide and/or fludarabine daily. In some aspect, the lymphodepletion therapy comprises cyclophosphamide and fludarabine daily. In some embodiments, the lymphodepletion therapy comprises cyclophosphamide at a concentration of about 300 mg/m² and fludarabine at a concentration of about 30 mg/m² daily for 3 days.

In some embodiments of the various methods or uses provided herein, the dose of the CAR T cells is 0.5-1.0×10⁶ cells/kg of body weight of the subject. In a preferred aspect, the dose of the CAR T cells is about 0.75×10⁶ cells/kg of body weight of the subject. In some embodiments, the method comprises administering the dose of the CAR T cells about 5 to about 7 days after the start of the lymphodepletion therapy. Preferably, the dose is administered as a single infusion. In some embodiments, a single intravenous infusion of CAR T cells of 0.75×10⁶ cells/kg is administered 5-7 days after the start of lymphodepletion.

Any of the anti-BCMA VHHs, CARs, and engineered immune effector cells (such as CAR-T cells) described herein may be used in the method of treating cancer. In some embodiments, the immune effector cells are autologous. In some embodiments, the immune effector cells are allogeneic.

In certain embodiments, the CAR-T cells are administered at a dose of about 1.0×10⁵ to 2.0×10⁵ cells/kg, 1.5×10⁵ to 2.5×10⁵ cells/kg, 2.0×10⁵ to 3.0×10⁵ cells/kg, 2.5×10⁵ to 3.5×10⁵ cells/kg, 3.0×10⁵ to 4.0×10⁵ cells/kg, 3.5×10⁵ to 4.5×10⁵ cells/kg, 4.0×10⁵ to 5.0×10⁵ cells/kg, 4.5×10⁵ to 5.5×10⁵ cells/kg, 5.0×10⁵ to 6.0×10⁵ cells/kg, 5.5×10⁵ to 6.5×10⁵ cells/kg, 6.0×10⁵ to 7.0×10⁵ cells/kg, 6.5×10⁵ to 7.5×10⁵ cells/kg, 7.0×10⁵ to 8.0×10⁵ cells/kg, 7.5×10⁵ to 8.5×10⁵ cells/kg, 8.0×10⁵ to 9.0×10⁵ cells/kg, 8.5×10⁵ to 9.5×10⁵ cells/kg, 9.0×10⁵ to 1.0×10⁵ cells/kg, 1.0×10⁶ to 2.0×10⁶ cells/kg, 1.5×10⁶ to 2.5×10⁶ cells/kg, 2.0×10⁶ to 3.0×10⁶ cells/kg, 2.5×10⁶ to 3.5×10⁶ cells/kg, 3.0×10⁶ to 4.0×10⁶ cells/kg, 3.5×10⁶ to 4.5×10⁶ cells/kg, 4.0×10⁶ to 5.0×10⁶ cells/kg, 4.5×10⁶ to 5.5×10⁶ cells/kg, or 5.0×10⁶ to 6.0×10⁶ cells/kg. In a preferred aspect, the dose comprises approximately 0.75×10⁶ cells/kg. In certain embodiments, the CAR-T cells are administered at a dose of about 1.0×10⁸ cells per subject. Preferably, the dose is administered as a single infusion.

In certain embodiments, the CAR-T cells are administered at a dose of less than 1.0×10⁸ cells per subject. In certain embodiments, the CAR-T cells are administered at a dose of about 3.0 to 4.0×10⁷ cells. In certain embodiments, the CAR-T cells are administered at a dose of about 3.5 to 4.5×10⁷ cells. In certain embodiments, the CAR-T cells are administered at a dose of about 4.0 to 5.0×10⁷ cells. In certain embodiments, the CAR-T cells are administered at a dose of about 4.5 to 5.5×10⁷ cells. In certain embodiments, the CAR-T cells are administered at a dose of about 5.0 to 6.0×10⁷ cells. In certain embodiments, the CAR-T cells are administered at a dose of about 5.5 to 6.5×10⁷ cells. In certain embodiments, the CAR-T cells are administered at a dose of about 6.0 to 7.0×10⁷ cells. In certain embodiments, the CAR-T cells are administered at a dose of about 6.5 to 7.5×10⁷ cells. In certain embodiments, the CAR-T cells are administered at a dose of about 7.0 to 8.0×10⁷ cells. In certain embodiments, the CAR-T cells are administered at a dose of about 7.5 to 8.5×10⁷ cells. In certain embodiments, the CAR-T cells are administered at a dose of about 8.0 to 9.0×10⁷ cells. In certain embodiments, the CAR-T cells are administered at a dose of about 8.5 to 9.5×10⁷ cells. In certain embodiments, the CAR-T cells are administered at a dose of about 9.0×10⁷ to 1.0×10⁸ cells.

In certain embodiments, the CAR-T cells are administered at a dose of about 0.693×10⁶ CAR-positive viable T-cells/kg. In certain embodiments, the CAR-T cells are administered at a dose of about 0.52×10⁶ CAR-positive viable T-cells/kg. In certain embodiments, the CAR-T cells are administered at a dose of about 0.94×10⁶ CAR-positive viable T-cells/kg. In certain embodiments, the CAR-T cells are administered at a dose of about 0.709×10⁶ CAR-positive viable T-cells/kg. In certain embodiments, the CAR-T cells are administered at a dose of about 0.51×10⁶ CAR-positive viable T-cells/kg. In certain embodiments, the CAR-T cells are administered at a dose of about 0.95×10⁶ CAR-positive viable T-cells/kg. In certain embodiments, the CAR-T cells are administered in an outpatient setting.

In some embodiments, the composition comprising CAR-T cells administered to the subject further comprises an excipient selected from dimethyl sulfoxide (DMSO) or dextran-40. In some embodiments, the composition comprises 5% DMSO.

In certain embodiments, the CAR-T cells (e.g., at any of the foregoing doses) are administered in one or more intravenous infusions. In certain embodiments, said administration of said CAR-T cells is via a single intravenous infusion. In certain embodiments, said single intravenous infusion is administered using a single bag of said CAR-T cells. In certain embodiments, said administration of said single bag of said CAR-T cells is completed between the time at which said single bag of CAR-T cells is thawed and three hours after said single bag of CAR-T cells is thawed. In certain embodiments, single intravenous administration is administered using two bags of said CAR-T cells. In certain embodiments, said administration of each of said two bags of said CAR-T cells is completed between the time at which a first bag of said two bags of CAR-T cells is thawed and three hours after said first bag of CAR-T cells is thawed.

In certain embodiments, the time since the initial apheresis to the administration of CAR-T cells is less than 41, 47, 54, 61, 68, 75, 82, 89, 96, 103, 110, 117, 124, 131, 138, 145, 152, 159, 166 or 167 days. In certain embodiments, the time since the initial apheresis to the administration of CAR-T cells is greater than 41, 47, 54, 61, 68, 75, 82, 89, 96, 103, 110, 117, 124, 131, 138, 145, 152, 159, 166 or 167 days.

In certain embodiments, a lymphodepleting regimen precedes the administration of CAR-T cells. In certain embodiments, the lymphodepleting regimen comprises administration of cyclophosphamide and/or administration of fludarabine. In certain embodiments, the lymphodepleting regimen is administered intravenously. In certain embodiments, the lymphodepleting regimen precedes the administration of CAR-T cells by 5 to 7 days. In certain embodiments, the lymphodepleting regimen precedes the administration of CAR-T cells by 2 to 4 days. In certain embodiments, the lymphodepleting regimen comprises intravenous administration of cyclophosphamide and fludarabine 5 to 7 days prior to the administration of CAR-T cells. In certain embodiments, the lymphodepleting regimen comprises intravenous administration of cyclophosphamide and fludarabine 2 to 4 days prior to the administration of CAR-T cells. In certain embodiments, the lymphodepleting regimen comprises cyclophosphamide administered intravenously at 300 mg/m². In certain embodiments, the lymphodepleting regimen comprises fludarabine administered intravenously at 30 mg/m². In some embodiments, the lymphodepleting regimen is performed daily for 3 days. In situations wherein the administration of the CAR-T cells is delayed by more than 14 days, the lymphodepleting regimen may be repeated.

In certain embodiments, the method of treatment with CAR-T cells further comprises treating the subject for cytokine release syndrome (CRS) within 3 days of CAR-T cell administration without significantly reducing CAR-T cell expansion in vivo. In certain embodiments, the treatment of CRS comprises administering the subject with an IL-6R inhibitor. In certain embodiments, the IL-6R inhibitor is an antibody. In certain embodiments, the IL-6 inhibitor inhibits IL-6R by binding its extracellular domain. In certain embodiments, the IL-6R inhibitor prevents the binding of IL-6 to IL-6R. In certain embodiments, the IL-6R inhibitor is tocilizumab. CRS can be identified based on clinical presentation. In some embodiments, other causes of fever, hypoxia and hypotension are evaluated and treated. Laboratory testing to monitor for disseminated intravascular coagulation, hematology parameters, as well as pulmonary, cardiac, renal, and hepatic function can be used. CRS can be managed according to the recommendations in Table 12. The methods can comprise administering anti-seizure prophylaxis with levetiracetam in patients who experience CRS. In some embodiments, the methods comprises monitoring patients who experience Grade 2 or higher CRS (e.g., hypotension not responsive to fluids, or hypoxia requiring supplemental oxygenation) with continuous cardiac telemetry and pulse oximetry. In some embodiments, intensive care unit level monitoring and supportive therapy can be used for severe or life-threatening CRS. For CRS refractory to first line interventions such as tocilizumab or tocilizumab and corticosteroids, the methods comprise alternate treatment options (i.e., higher corticosteroid dose, alternative anti-cytokine agents, e.g. anti-IL1 and/or anti-TNFα, anti-T cell therapies). Refractory CRS is characterized by fevers, end-organ toxicity (e.g., hypoxia, hypotension) not improving within 12 hours of first line interventions or development of HLH/MAS.

In certain embodiments, the method of treatment with CAR-T cells further comprises treating the subject with pre-infusion medication comprising an antipyretic and an antihistamine up to 1 hour prior to the administration of CAR-T cells. In certain embodiments, the antipyretic comprises either paracetamol or acetaminophen. In certain embodiments, the antipyretic is administered to the subject either orally or intravenously. In certain embodiments, the antipyretic is administered to the subject at a dosage of between 650 mg and 1000 mg. In certain embodiments, the antihistamine comprises diphenhydramine. In certain embodiments, the antihistamine is administered to the subject either orally or intravenously. In certain embodiments, the antihistamine is administered at a dosage of between 25 mg and 50 mg, or its equivalent. The composition comprising the host cells expressing the CAR-encoding nucleic acid sequences disclosed herein, or a vector comprising the CAR-encoding nucleic acid sequences disclosed herein, can be administered to a mammal using standard administration techniques, including oral, intravenous, intraperitoneal, subcutaneous, pulmonary, transdermal, intramuscular, intranasal, buccal, sublingual, or suppository administration. The composition preferably is suitable for parenteral administration. The term “parenteral”, as used herein, includes intravenous, intramuscular, subcutaneous, rectal, vaginal, and intraperitoneal administration. More preferably, the composition is administered to a mammal using peripheral systemic delivery by intravenous, intraperitoneal, or subcutaneous injection. Most preferably, the composition is administered by intravenous infusion.

The composition comprising the host cells expressing the CAR-encoding nucleic acid sequences disclosed herein, or a vector comprising the CAR-encoding nucleic acid sequences disclosed herein, can be administered with one or more additional therapeutic agents, which can be coadministered to the mammal. By “coadministering” is meant administering one or more additional therapeutic agents and the composition comprising the host cells disclosed herein or the vectors disclosed herein sufficiently close in time such that the CARs disclosed herein can enhance the effect of one or more additional therapeutic agents, or vice versa. In this regard, the composition comprising the host cells disclosed herein or the vectors disclosed herein can be administered first, and the one or more additional therapeutic agents can be administered second, or vice versa.

A CAR-expressing cell described herein and the at least one additional therapeutic agent can be administered simultaneously, in the same or in separate compositions, or sequentially. For sequential administration, the CAR-expressing cell described herein can be administered first, and the additional agent can be administered second, or the order of administration can be reversed.

In certain embodiments, a lymphodepleting regimen precedes the administration of CAR-T cells. In certain embodiments, the lymphodepleting regimen precedes said administration of CAR-T cells by approximately 2 days to approximately 7 days. In certain embodiments, lymphodepleting regimen is administered intravenously. In certain embodiments, said lymphodepleting regimen comprises administration of cyclophosphamide or administration of fludarabine. In certain embodiments, said cyclophosphamide is administered intravenously at 300 mg/m². In certain embodiments, said fludarabine is administered intravenously at 30 mg/m².

In certain embodiments, a lymphodepleting regimen comprising cyclophosphamide administered intravenously at 300 mg/m² and fludarabine administered intravenously at 30 mg/m² precedes said administration of CAR-T cells by approximately 2 days to approximately 7 days.

In certain embodiments, the subject further receives bridging therapy, wherein said bridging therapy comprises short-term treatment with at least one bridging medicament between apheresis and said lymphodepleting regimen, and wherein said at least one bridging medicament had previously obtained an outcome of stable disease, minimal response, partial response, very good partial response, complete response or stringent complete response for the subject. In certain embodiments, the subject had an increase in tumor burden despite said bridging therapy. In certain embodiments, the subject had an increase in tumor burden of approximately 25% or greater despite said bridging therapy. Suitable bridging therapies include, for example, dexamethasone, daratumumab, bortezomib, cyclophosphamide, and pomalidomide. In some embodiments, the bridging therapy comprises pomalidomide, bortezomib, dexamethasone, daratumumab, or any combination thereof. In some embodiments, the bridging therapy comprises dexamethasone. In some embodiments, the bridging therapy comprises daratumumab. In some embodiments, the bridging therapy comprises bortezomib. In some embodiments, the bridging therapy comprises cyclophosphamide. In some embodiments, the bridging therapy comprises pomalidomide. In some embodiments, the bridging therapy comprises pomalidomide, bortezomib and dexamethasone. In some embodiments, the bridging therapy comprises daratumumab, pomalidomide and dexamethasone. In some embodiments, the subject has received the bridging therapy from about every 10 days to about every 40 days. In some embodiments, the subject has received the bridging therapy from about every 20 days to about every 30 days. In some embodiments, the subject has received the bridging therapy about every 21 days. In some embodiments, the subject has received the bridging therapy about every 15 days. In some embodiments, the subject has received the bridging therapy about every 25 days. In some embodiments, the subject has received the bridging therapy about every 21 days. In some embodiments, the subject has received the bridging therapy about every 28 days. In some embodiments, the subject has received the bridging therapy about every 30 days. In some embodiments, the subject has received the bridging therapy about every 35 days. In some embodiments, the subject has received at least one, two, three, four, or more bridging therapies. In some embodiments, the subject has received at least one bridging therapy. In some embodiments, the subject has received at least two bridging therapies. In some embodiments, the subject has received at least three bridging therapies. In some embodiments, the subject has received at least four bridging therapies. In some embodiments, the subject has received at least five bridging therapies. In some embodiments, the subject has received at least six bridging therapies.

In certain embodiments, the subject is treated with pre-administration medication comprising an antipyretic and an antihistamine up to approximately 1 hour before said administration of said CAR-T cells. In certain embodiments, said antipyretic comprises either paracetamol or acetaminophen. In certain embodiments, said antipyretic is administered to the subject either orally or intravenously. In certain embodiments, said antipyretic is administered to the subject at a dosage of between 650 mg and 1000 mg. In certain embodiments, said antihistamine comprises diphenhydramine. In certain embodiments, said antihistamine is administered to the subject either orally or intravenously. In certain embodiments, said antihistamine is administered at a dosage of between 25 mg and 50 mg, or its equivalent. In certain embodiments, said antipyretic comprises either paracetamol or acetaminophen and said antipyretic is administered to the subject either orally or intravenously at a dosage of between 650 mg and 1000 mg, and wherein said antihistamine comprises diphenhydramine and said antihistamine is administered to the subject either orally or intravenously at a dosage of between 25 mg and 50 mg, or its equivalent.

In some embodiments, the methods comprise, prior to administration of the CAR-T cells, administering a lymphodepleting chemotherapy regimen comprising cyclophosphamide 300 mg/m² intravenously (IV) and fludarabine 30 mg/m² IV daily for 3 days, and administering pre-infusion medications comprising an antipyretic (such as oral or intravenous acetaminophen 650 to 1000 mg) and antihistamine (such as oral or intravenous diphenhydramine 25 to 50 mg or equivalent), wherein:
the CAR-T cells are administered 2-4 days after completion of the lymphodepleting chemotherapy and
the CAR-T cells are administered 30-60 minutes after the administration of the pre-infusion medications.

In some embodiments, the CAR-T cells are not administered, or the administration of the CAR-T cells is delayed, if the patient has any of the following conditions: clinically significant active infection or inflammatory disorder; or Grade ≥3 non-hematologic toxicities of cyclophosphamide and fludarabine conditioning, except for Grade 3 nausea, vomiting, diarrhea, or constipation. Administration of the CAR-T cells should be delayed until resolution of these events to Grade ≤1. In some embodiments, prophylactic systemic corticosteroids are not administered.

In some embodiments, the method further comprises diagnosing said subject for cytokine release syndrome (CRS). In preferred embodiments, the diagnosis is made according to the American Society of Transplantation and Cellular Therapy (ASTCT), formerly the American Society for Blood and Marrow Transplantation (ASBMT) consensus grading. A non-limiting summary of the ASTCT consensus grading for CRS diagnosis is provided in Table 13.

In some embodiments, the method further comprises treating said subject for cytokine release syndrome (CRS). In some embodiments, the treatment of CRS is with an antipyretic. In some examples, the treatment of CRS is with anticytokine therapy. In some embodiments, the treatment of CRS occurs more than approximately 3 days following the infusion. In some embodiments, the treatment of CRS occurs without significantly reducing CAR-T cell expansion in vivo. In certain embodiments, said method further comprises treating said subject for cytokine release syndrome more than approximately 3 days following said administration of said CAR-T cells without significantly reducing expansion of said CAR-T cells in vivo. In some embodiments, the treatment of CRS comprises administering to the subject an IL-6R inhibitor. In some embodiments, the IL-6R inhibitor is an antibody. In some embodiments, the antibody inhibits IL-6R by binding its extracellular domain. In some embodiments, the IL-6R inhibitor prevents the binding of IL-6 to IL-6R. In some embodiments, the IL-6R inhibitor is tocilizumab. In some embodiments, the anticytokine therapy comprises administration of tocilizumab. In some embodiments, the anticytokine therapy comprises administration of steroids. In some embodiments, treatment for CRS comprises treatment with monoclonal antibodies other than tocilizumab. In some embodiments, the antibodies other than tocilizumab target cytokines. In some embodiments, the cytokine that the antibodies other than tocilizumab target is IL-1. In some embodiments, the IL-1 targeting antibody is Anakinra. In some embodiments, the cytokine that the antibodies other than tocilizumab target is TNFα. In some embodiments, the treatment of CRS comprises administering to the subject a corticosteroid. In some embodiments, the treatment of CRS comprises using a vasopressor. In some embodiments, the treatment of CRS comprises intubation or mechanical ventilation. In some embodiments, the treatment of CRS comprises administering to the subject cyclophosphamide. In some embodiments, the treatment of CRS comprises administering to the subject etanercept. In some embodiments, the treatment of CRS comprises administering to the subject levetiracetam. In some embodiments, the treatment of CRS comprises supportive care.

In some embodiments, the method further comprises diagnosing said subject for immune cell effector-associated neurotoxicity (ICANS). In some embodiments, the diagnosis is made according to the National Cancer Institute Common Terminology Criteria for Adverse Events (NCI CTCAE) criteria. In some embodiments, the diagnosis is made according to the NCI CTCAE criteria, Version 5.0. In some embodiments, the diagnosis is made according to the American Society of Transplantation and Cellular Therapy (ASTCT) consensus grading system. In some embodiments, the embodiments, there is neurotoxicity consistent with ICAN. A non-limiting summary of the ASTCT consensus grading system for ICANS diagnosis is provided in Table 14. In some embodiments, the treatment of ICANS comprises administering to the subject an IL-6R inhibitor. In some embodiments, the IL-6R inhibitor is an antibody. In some embodiments, the antibody inhibits IL-6R by binding its extracellular domain. In some embodiments, the IL-6R inhibitor prevents the binding of IL-6 to IL-6R. In some embodiments, the IL-6R inhibitor is tocilizumab. In some embodiments, the treatment of ICANS comprises administering to the subject an IL-1 inhibitor. In some embodiments the IL-1 inhibitor is an antibody. In a preferred aspect, the IL-1 inhibiting antibody is Anakinra. In some embodiments, the treatment of ICANS comprises administering to the subject a corticosteroid. In some embodiments, the treatment of ICANS comprises administering to the subject levetiracetam. In some embodiments, the treatment of ICANS comprises administering to the subject dexamethasone. In some embodiments, the treatment of ICANS comprises administering to the subject methylprednisone sodium succinate. In some embodiments, the treatment of ICANS comprises administering to the subject pethidine. In some embodiments, the treatment of ICANS comprises administering to the subject one or more of, or all of, tocilizumab, Anakinra, a corticosteroid, levetiracetam, dexamethasone, methylprednisone sodium succinate or pethidine.

If concurrent neurologic toxicity is suspected during CRS or vice versa, the methods can comprise administering:
Corticosteroids according to the more aggressive intervention based on the CRS and neurologic toxicity grades in Tables 1 and 2 of the Approved Label.
Tocilizumab according to the CRS grade in Table 1 of the Approved Label.
Anti-seizure medication according to the neurologic toxicity in Table 2 of the Approved Label.

In some embodiments, the method further comprises diagnosing said subject for cytopenias. In some embodiments, the cytopenias comprise one or more of, or all of, lymphopenia, neutropenia, and thrombocytopenia. Without being bound by theory, a Grade 3 or Grade 4 but not a Grade 2 or lower lymphopenia is characterized by to a lymphocyte count less than 0.5×10⁹ cells per liter of a subject's blood sample, a Grade 3 or Grade 4 but not a Grade 2 or lower neutropenia is characterized by a neutrophil count less than 1000 cells per microliter of a subject's blood sample, and a Grade 3 or Grade 4 but not a Grade 2 or lower thrombocytopenia is characterized by a platelet count less than 50,000 cells per microliter of a subject's blood sample. In some embodiments, greater than 75% of subjects with Grade 3 or Grade 4 lymphopenia following CAR-T cell administration recover to Grade 2 or lower lymphopenia 60 days following CAR-T cell administration. In some embodiments, greater than 80% of subjects with Grade 3 or Grade 4 lymphopenia following CAR-T cell administration recover to Grade 2 or lower lymphopenia 60 days following CAR-T cell administration. In some embodiments, greater than 85% of subjects with Grade 3 or Grade 4 lymphopenia following CAR-T cell administration recover to Grade 2 or lower lymphopenia 60 days following CAR-T cell administration. In some embodiments, greater than 90% of subjects with Grade 3 or Grade 4 lymphopenia following CAR-T cell administration recover to Grade 2 or lower lymphopenia 60 days following CAR-T cell administration. In some embodiments, greater than 70% of subjects with Grade 3 or Grade 4 neutropenia following CAR-T cell administration recover to Grade 2 or lower neutropenia 60 days following CAR-T cell administration. In some embodiments, greater than 75% of subjects with Grade 3 or Grade 4 neutropenia following CAR-T cell administration recover to Grade 2 or lower neutropenia 60 days following CAR-T cell administration. In some embodiments, greater than 80% of subjects with Grade 3 or Grade 4 neutropenia following CAR-T cell administration recover to Grade 2 or lower neutropenia 60 days following CAR-T cell administration. In some embodiments, greater than 85% of subjects with Grade 3 or Grade 4 neutropenia following CAR-T cell administration recover to Grade 2 or lower neutropenia 60 days following CAR-T cell administration. In some embodiments, greater than 30% of subjects with Grade 3 or Grade 4 thrombocytopenia following CAR-T cell administration recover to Grade 2 or lower thrombocytopenia 60 days following CAR-T cell administration. In some embodiments, greater than 34% of subjects with Grade 3 or Grade 4 thrombocytopenia following CAR-T cell administration recover to Grade 2 or lower thrombocytopenia 60 days following CAR-T cell administration. In some embodiments, greater than 38% of subjects with Grade 3 or Grade 4 thrombocytopenia following CAR-T cell administration recover to Grade 2 or lower thrombocytopenia 60 days following CAR-T cell administration. In some embodiments, greater than 42% of subjects with Grade 3 or Grade 4 thrombocytopenia following CAR-T cell administration recover to Grade 2 or lower thrombocytopenia 60 days following CAR-T cell administration.

Once the composition comprising host cells expressing the CAR-encoding nucleic acid sequences disclosed herein, or a vector comprising the CAR-encoding nucleic acid sequences disclosed herein, is administered to a mammal (e.g., a human), the biological activity of the CAR can be measured by any suitable method known in the art. In accordance with the methods disclosed herein, the CAR binds to BCMA on the multiple myeloma cells, and the multiple myeloma cells are destroyed. Binding of the CAR to BCMA on the surface of multiple myeloma cells can be assayed using any suitable method known in the art, including, for example, ELISA and flow cytometry. The ability of the CAR to destroy multiple myeloma cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). The biological activity of the CAR also can be measured by assaying expression of certain cytokines, such as CD 107a, IFNγ, IL-2, and TNF.

The methods described herein may be used for treating various cancers, including both solid cancer and liquid cancer. In certain embodiments, the methods are used to treat multiple myeloma. The methods described herein may be used as a first therapy, second therapy, third therapy, or combination therapy with other types of cancer therapies known in the art, such as chemotherapy, surgery, radiation, gene therapy, immunotherapy, bone marrow transplantation, stem cell transplantation, targeted therapy, cryotherapy, ultrasound therapy, photodynamic therapy, radio-frequency ablation or the like, in an adjuvant setting or a neoadjuvant setting.

In certain embodiments, the cancer is multiple myeloma. In certain embodiments, the cancer is stage I, stage II or stage III, and/or stage A or stage B multiple myeloma based on the Durie-Salmon staging system. In certain embodiments, the cancer is stage I, stage II or stage III multiple myeloma based on the International staging system published by the International Myeloma Working Group (IMWG). In some embodiments, the multiple myeloma is progressive.

In certain aspects, the subject received prior treatment with one or more line of therapy. In some embodiments, the number of lines of prior therapies is 1. In certain embodiments, the number of lines of prior therapy is 2. In some embodiments, the number of lines of prior therapies is 3. In some embodiments, the number of lines of prior therapies is 4. In some embodiments, the number of lines of prior therapies is 5. In certain embodiments, prior lines of therapy include surgery, radiotherapy, or autologous or allogeneic transplant, or any combination of such treatments. In certain embodiments, the prior treatment comprises treatment with a medicament that is a proteasomal inhibitor (PI). Non-limiting examples of a PI include bortezomib, carfilzomib and ixazomib. In certain embodiments, the prior treatment comprises treatment with a medicament that is an immunomodulatory drug (IMiD). Non-limiting examples of an IMiD include lenalidomide, pomalidomide and thalidomide. In preferred embodiments, the prior treatment comprises treatment with lenalidomide, optionally treatment comprising lenalidomide and a proteasome inhibitor. In preferred embodiments, the subject received at least one prior line of therapy comprising lenalidomide, optionally comprising lenalidomide and a proteasome inhibitor, and optionally received one or two further prior lines of therapy. In certain embodiments, the prior treatment comprises treatment with a medicament that is a corticosteroid. Non-limiting examples of a corticosteroid include dexamethasone and prednisone. In certain embodiments, the prior treatment comprises treatment with a medicament that is an alkylating agent. In certain embodiments, the prior treatment comprises treatment with a medicament that is an anthracycline. In certain embodiments, the prior treatment comprises treatment with a medicament that is an anti-CD38 antibody. Non-limiting examples of an anti-CD38 antibody include daratumumab, isatuximab and the investigational antibody TAK-079. In certain embodiments, the prior treatment comprises treatment with a medicament that is elotuzumab. In certain embodiments, the prior treatment comprises treatment with a medicament that is panobinostat. In certain embodiments, the prior treatment comprises treatment with at least one medicament, said at least one medicament comprising at least one of a proteasome inhibitor (PI), an IMiD, and/or an anti-CD38 antibody. In certain embodiments, the prior line of therapy comprises treatment with at least one medicament, said at least one medicament comprising of at least one of PI, an IMiD, and/or an alkylating agent. In certain embodiments, the subject has relapsed after the prior line of therapy.

In certain embodiments, the multiple myeloma is refractory to one or more of, or all of, bortezomib, carfilzomib, ixazomib, lenalidomide, pomalidomide, thalidomide, dexamethasone, prednisone, alkylating agents, daratumumab, isatuximab, TAK-079, elotuzumab and/or panobinostat. In certain embodiments, the multiple myeloma is refractory to at least one medicament following the one or more prior lines of therapy. In certain embodiments, the at least one medicament to which the multiple myeloma is refractory comprise an IMiD. In some embodiments, the IMiD comprises lenalidomide, pomalidomide or thalidomide. In some embodiments, the IMiD comprises lenalidomide. In preferred embodiments, the multiple myeloma is lenalidomide-refractory multiple myeloma. In some embodiments, the IMiD is lenalidomide. In certain embodiments, the multiple myeloma is refractory to at least two medicaments following the prior of therapy. In certain embodiments, the at least two medicaments to which the multiple myeloma is refractory comprise a proteasome inhibitor (PI) and an IMiD (e.g. lenalidomide). In certain embodiments, the multiple myeloma is refractory to at least three medicaments following the prior line of therapy. In certain embodiments, the multiple myeloma is refractory to at least four medicaments following the prior line of therapy. In certain embodiments, the at least four prior lines of therapy comprise treatment with at least one medicament, said at least one medicament comprising of at least one of PI, an IMiD, anti-CD38 antibody, and/or an alkylating agent. In certain embodiments, the multiple myeloma is refractory to at least five medicaments following the prior line of therapy.

In some embodiments, the subject has bone marrow plasma cells of between approximately 10% and approximately 30% before said administration of said CAR-T cells.

In certain embodiments, bone marrow aspirate or biopsy may be performed for clinical assessments or bone marrow aspirate may be performed for biomarker evaluations. In certain embodiments, clinical staging (morphology, cytogenetics, and immunohistochemistry or immunofluorescence or flow cytometry) may be done. In certain embodiments, a portion of the bone marrow aspirate may be immunophenotyped and monitored for BCMA, checkpoint ligand expression in CD138-positive multiple myeloma cells, and checkpoint expression on T cells. In certain embodiments, minimal residual disease (MRD) may be monitored in subjects using next generation sequencing (NGS) of bone marrow aspirate DNA. The NGS of bone marrow aspirate DNA is known to one of ordinary skill in the art. In certain embodiments, the NGS is performed via clonoSeq. In certain embodiments, baseline bone marrow aspirates may be used to define the myeloma clones, and post-treatment samples may be used to evaluate MRD negativity. In certain embodiments, the MRD negativity status may be based on samples that are evaluable. In certain embodiments, evaluable samples are those that passed one or more of, or all of, calibration, quality control, and sufficiency of cells evaluable at a particular sensitivity level. In some embodiments, the sensitivity level is 10⁻⁶. In certain embodiments, the sensitivity level is 10⁻⁶, the sensitivity level is 10⁻⁵. In certain embodiments, the sensitivity level is 10⁻⁴. In certain embodiments, the sensitivity level is 10⁻³.

In certain embodiments, a subject's response to the method of treatment is assessed using the International Myeloma Working Group (IMWG)-based response criteria, which are summarized in Table 6. In certain embodiments, the response may be classified as a stringent complete response (sCR). In certain embodiments, the response may be classified as a complete response (CR), which is worse than a stringent complete response (sCR). In certain embodiments, the response may be classified as a very good partial response (VGPR), which is worse than a complete response (CR). In certain embodiments, the response may be classified as a partial response (PR), which is worse than a very good partial response (VGPR). In certain embodiments, the response may be classified as a minimal response (MR), which is worse than a partial response (PR). In certain embodiments, the response may be classified as a stable disease (SD), which is worse than a minimal response (MR). In certain embodiments, the response may be classified as a progressive disease (PD), which is worse than a stable disease.

In certain embodiments, the tests used to assess International Myeloma Working Group (IMWG)-based response criteria are Myeloma protein (M-protein) measurements in serum and urine, serum calcium corrected for albumin, bone marrow examination, skeletal survey and documentation of extramedullary plasmacytomas.

Non-limiting examples of tests for M-protein measurement in blood and urine are known to one of ordinary skill in the art and comprise serum quantitative Ig, serum protein electrophoresis (SPEP), serum immunofixation electrophoresis, serum FLC assay, 24-hour urine M-protein quantitation by electrophoresis (UPEP), urine immunofixation electrophoresis, and serum β2-microglobulin.

Calculating serum calcium corrected for albumin in blood samples for detection of hypercalcemia is known to one of ordinary skill in the art. Without wishing to be bound by theory, calcium binds to albumin and only the unbound (free) calcium is biologically active; therefore, the serum calcium level must be adjusted for abnormal albumin levels (“corrected serum calcium”).

In certain embodiments, a skeletal survey of any one of, or all of, the skull, the entire vertebral column, the pelvis, the chest, the humeri, the femora, and any other bones, may be performed and evaluated by either roentgenography (“X-rays”) or low-dose computed tomography (CT) diagnostic quality scans without the use of IV contras, both of which are known to one of ordinary skill in the art. In certain embodiments, following T cell administration and before disease progression is confirmed, X-rays or CT scans may be performed locally, whenever clinically indicated based on symptoms, to document response or progression. In certain embodiments, magnetic resonance imaging (MRI) may be used for evaluating bone disease but does not replace a skeletal survey. MRI is known to one of ordinary skill in the art. In certain embodiments, if a radionuclide bone scan is used at screening, in addition to the complete skeletal survey, both methods may be used to document disease status. Radionuclide bone scans are known to one of ordinary skill in the art. In certain embodiments, the radionuclide bone scan and complete skeletal survey may be performed at the same time. In certain embodiments, a radionuclide bone scan may not replace a complete skeletal survey. In certain embodiments, if a subject presents with disease progression manifested by symptoms of pain due to bone changes, then disease progression may be documented by skeletal survey or other radiographs, depending on the symptoms that the subject experiences.

In certain embodiments, extramedullary plasmacytomas may be documented by clinical examination or MRI. In certain embodiments, if there was no contraindication to the use of IV contrast, extramedullary plasmacytomas may be documented by CT scan. In certain embodiments, extramedullary plasmacytomas may be documented by a fusion of positron emission tomography (PET) and CT scans if the CT component is of sufficient diagnostic quality. In certain embodiments, assessment of measurable sites of extramedullary disease may be performed, measured, or evaluated locally every 4 weeks for subjects until development of confirmed CR or confirmed disease progression. In certain embodiments, evaluation of extramedullary plasmacytomas may be done every 12 weeks.

In certain embodiments, to qualify for VGPR or PR or MR, the sum of products of the perpendicular diameters of the existing extramedullary plasmacytomas may have decreased by over 90% or at least 50%, respectively. In certain embodiments, to qualify for disease progression, either the sum of products of the perpendicular diameters of the existing extramedullary plasmacytomas must have increased by at least 50%, or the longest diameter of previous lesion >1 cm in short axis must have increased at least 50%, or a new plasmacytoma must have developed. In certain embodiments, to qualify for disease progression when not all existing extramedullary plasmacytomas are reported, the sum of products of the perpendicular diameters of the reported plasmacytomas had increased by at least 50%. In certain embodiments, if the study treatment interferes with the immunofixation assay, CR may be defined as the disappearance of the original M-protein associated with multiple myeloma on immunofixation.

In certain embodiments, a subject's response to the method of treatment is assessed in terms of change in disease burden or tumor burden. Disease burden or tumor burden represents the type of measurable disease in the subject. In some embodiments, the change in tumor burden may be assessed in terms of paraprotein level changes upon treatment. In some embodiments, the paraprotein is an M-protein in the serum. In some embodiments, the paraprotein is an M-protein in the serum. In some embodiments, the change in tumor burden is assessed in terms of the difference between involved and uninvolved free light chain (dFLC). In some embodiments, the change in tumor burden is assessed in terms of the maximum paraprotein reduction from baseline, i.e., from prior to the administration of the CAR-T cells. In some embodiments, the change in tumor burden is assessed at a median follow-up time of greater than or equal to 28 days following the administration of CAR-T cells. In some embodiments, the change in tumor burden is assessed at a median follow-up time of greater than or equal to 1 month following the administration of CAR-T cells. In some embodiments, the change in tumor burden is assessed at a median follow-up time of greater than or equal to 3 months following the administration of CAR-T cells. In some embodiments, the change in tumor burden is assessed at a median follow-up time of greater than or equal to 6 months following the administration of CAR-T cells. In some embodiments, the change in tumor burden is assessed at a median follow-up time of greater than or equal to 9 months following the administration of CAR-T cells. In some embodiments, the change in tumor burden is assessed at a median follow-up time of greater than or equal to 12 months following the administration of CAR-T cells.

In certain embodiments, the subject is re-treated by administration via a second intravenous infusion of a second dose of CAR-T cells. In certain embodiments, the re-treatment dose comprises 1.0×10⁵ to 5.0×10⁶ of CAR-T cells per kilogram of the mass of the subject. In certain embodiments, the re-treatment dose comprises approximately 0.75×10⁵ of CAR-T cells per kilogram of the mass of the subject. In certain embodiments, the subject is re-treated upon exhibiting progressive disease after a best response of minimal response or better following the first infusion of CAR-T cells. In certain embodiments, the time between the first infusion of CAR-T cells and the detection of the progressive disease comprises at least six months.

In one aspect is provided a method of treating a subject who has multiple myeloma, said method comprising administering to the subject via a single intravenous infusion a composition comprising a therapeutically effective number of T cells comprising a chimeric antigen receptor (CAR) to deliver to the subject a dose of CAR expressing T cells (CAR-T cells).

In some embodiments, the subject received prior treatment with at least one to three prior lines of therapy. In some embodiments, the prior line of therapy comprises treatment with at least one medicament, said at least one medicament comprising of at least one of a proteasome inhibitor (PI), an IMiD, and an anti-CD38 antibody. In some embodiments, the subject has relapsed after the prior line of therapy.

In some embodiments, the multiple myeloma is refractory to at least two medicaments following the prior line of therapy. In some embodiments, said at least two medicaments to which the subject is refractory comprise proteasome inhibitor (PI) and an IMiD (e.g. lenalidomide). In some embodiments, the multiple myeloma is refractory to at least three medicaments following the prior line of therapy. In some embodiments, the multiple myeloma is refractory to at least four medicaments following the prior line of therapy. In some embodiments, the multiple myeloma is refractory to at least five medicaments following the prior line of therapy.

In some embodiments, the subject is greater than 65 years of age. In some embodiments, the subject is Black or African American. In some embodiments, the subject has received 1-3 prior lines of therapy. In some embodiments, the subject has received at least 1 prior line of therapy. In some embodiments, the subject has received at least 2 prior lines of therapy. In some embodiments, the subject has received at least 3 prior lines of therapy. In some embodiments, the subject has received at least 4 prior lines of therapy. In some embodiments, the multiple myeloma or the subject is refractory to three classes of medicaments, i.e., the multiple myeloma or the subject is triple-class refractory. In some embodiments, the multiple myeloma or the subject is refractory to five medicaments or drugs, i.e., the multiple myeloma or the subject is penta-drug refractory. In some embodiments, the subject has high-risk disease factors, including high-risk cytogenetic abnormality, soft tissue plasmacytomas, triple-class-refractory, or other high-risk disease factors. In some embodiments, the subject has standard-risk cytogenetics. In some embodiments, the subject has high-risk cytogenetics. In some embodiments, the cytogenetic abnormality is a standard-risk cytogenetic abnormality. In some embodiments, the cytogenetic abnormality is a high-risk cytogenetic abnormality. In some embodiments, the subject has one or more high-risk cytogenetic abnormality selected from a group comprising Gain/amp(1q), del(17p), t(4;14), t(14;16), or any combination thereof. In some embodiments, the cytogenetic abnormality comprises Gain/amp(1q). In some embodiments, the cytogenetic abnormality comprises del(17p). In some embodiments, the cytogenetic abnormality comprises t(4;14). In some embodiments, the cytogenetic abnormality comprises t(14;16). In some embodiments, the subject has two, three, four, five, or more cytogenetic abnormalities. In some embodiments, the subject has at least two cytogenetic abnormalities. In some embodiments, the subject has at least three cytogenetic abnormalities. In some embodiments, the subject has at least four cytogenetic abnormalities. In some embodiments, the subject has at least five cytogenetic abnormalities. In some embodiments, the subject has at least six cytogenetic abnormalities. In some embodiments, the subject has at least seven cytogenetic abnormalities. In some embodiments, the subject or multiple myeloma has been characterized as stage III per the International Staging System. In some embodiments, the subject has soft tissue plasmacytomas. In some embodiments, the subject has bone marrow plasma cells of between approximately 10% and approximately 30% before said administration of said CAR-T cells. In some embodiments, the subject has bone marrow plasma cells of between approximately 31% and approximately 59% before said administration of said CAR-T cells. In some embodiments, the subject has bone marrow plasma cells of between approximately 60% and approximately 100% before said administration of said CAR-T cells. In some embodiments, the subject has BCMA expression in the tumor less than the median in a population of multiple myeloma patients, or in any randomly selected population. In some embodiments, the subject has BCMA expression in the tumor greater than or equal to the median in a population of multiple myeloma patients, or in any randomly selected population. In some embodiments, plasmacytomas are present in the subject. In some embodiments, the plasmacytomas are bone based. In some embodiments, the plasmacytomas are extramedullary. In some embodiments, the plasmacytomas are both bone based and extramedullary.

In certain embodiments, the method of treatment is effective in obtaining in the subject a reduction in tumor burden. In certain embodiments, the method of treatment is effective in obtaining in the subject a reduction in tumor burden of between approximately between approximately 1% and approximately 100%, between approximately 60% and approximately 100%, between approximately 65% and approximately 100%, between approximately 70% and approximately 100%, between approximately 75% and approximately 100%, between approximately 80% and approximately 100%, between approximately 85% and approximately 100%, between approximately 90% and approximately 100%, between approximately 92% and approximately 100%, between approximately 95% and approximately 100%, between approximately 96% and approximately 100%, between approximately 97% and approximately 100%, between approximately 98% and approximately 100%, or between approximately 99% and approximately 100%. In certain embodiments, the method of treatment is effective in obtaining in the subject a reduction in tumor burden of approximately 100%. In certain embodiments, the method of treatment is effective in obtaining in the subject a reduction in tumor burden of between approximately 1% and approximately 100% at a rate of between approximately 1% and approximately 100%. In certain embodiments, the method of treatment is effective in obtaining in the subject a reduction in tumor burden of between approximately 60% and approximately 100% at a rate of between approximately 1% and approximately 100%. In certain embodiments, the method of treatment is effective in obtaining in the subject a reduction in tumor burden of between approximately 65% and approximately 100% at a rate of between approximately 1% and approximately 92%. In certain embodiments, the method of treatment is effective in obtaining in the subject a reduction in tumor burden of between approximately 70% and approximately 100% at a rate of between approximately 1% and approximately 88%. In certain embodiments, the method of treatment is effective in obtaining in the subject a reduction in tumor burden of between approximately 90% and approximately 100% at a rate of between approximately 1% and approximately 88%. In certain embodiments, the method of treatment is effective in obtaining in the subject a reduction in tumor burden of between approximately 95% and approximately 100% at a rate of between approximately 1% and approximately 88%. In certain embodiments, the method of treatment is effective in obtaining in the subject a reduction in tumor burden of between approximately 99% and approximately 100% at a rate of between approximately 1% and approximately 88%. In certain embodiments, the method of treatment is effective in obtaining in the subject a reduction in tumor burden of approximately 100% at a rate of between approximately 1% and approximately 83%.

In certain embodiments, the method of treatment is effective in obtaining in the subject minimal residual disease (MRD) negative status or maintaining said minimal residual disease (MRD) status. In certain embodiments, the method of treatment is effective in obtaining in the subject a minimal residual disease (MRD) negative status at a sensitivity level of 10⁻⁶. In certain embodiments, the method of treatment is effective in obtaining in the subject minimal residual disease (MRD) negative status at a sensitivity level of 10⁻⁵. In certain embodiments, the method of treatment is effective in obtaining in the subject minimal residual disease (MRD) negative status at a sensitivity level of 10⁻⁴. In certain embodiments, the method of treatment is effective in obtaining in the subject minimal residual disease (MRD) negative status at a sensitivity level of 10⁻³. In certain embodiments, the method of treatment is effective in obtaining MRD negative status when assessed in the bone marrow. In certain embodiments, the method of treatment is effective in maintaining the MRD negative status when assessed using a bone marrow sample that is evaluable. In certain embodiments, the method of treatment is effective in obtaining MRD negative status when assessed using bone marrow DNA. In some embodiments, said method is effective in obtaining minimal residual disease (MRD) negative status in said subject assessed in the bone marrow at a follow-up time of approximately 28 days or later after said administration of said CAR-T cells, approximately 2 months or later after said administration of said CAR-T cells, approximately 3 months or later after said administration of said CAR-T cells, approximately 6 months or later after said administration of said CAR-T cells, approximately 9 months or later after said administration of said CAR-T cells, or approximately 12 months or later after said administration of said CAR-T cells. In some embodiments, said minimal residual disease (MRD) negative status is obtained at a first follow-up time of between approximately 28 days and approximately 179 days after said infusion of said CAR-T cells.

In certain embodiments, the method of treatment is effective in maintaining in the subject a first obtained minimal residual disease (MRD) negative status. In certain embodiments, the method of treatment is effective in maintaining MRD negative status at a sensitivity level of 10⁻⁵. In certain embodiments, the method of treatment is effective in obtaining in the subject minimal residual disease (MRD) negative status at a sensitivity level of 10⁻⁶. In certain embodiments, the method of treatment is effective in maintaining MRD negative status at a sensitivity level of 10⁴. In certain embodiments, the method of treatment is effective in maintaining MRD negative status at a sensitivity level of 10⁻³. In certain embodiments, the method of treatment is effective in maintaining the MRD negative status when assessed using a bone marrow sample. In certain embodiments, the method of treatment is effective in maintaining the MRD negative status when assessed using a bone marrow sample that is evaluable. In certain embodiments, the method of treatment is effective in maintaining MRD negative status is maintained when assessed using bone marrow DNA. In some embodiments, said method is effective in maintaining said minimal residual disease (MRD) negative status in said subject assessed in the bone marrow at a second follow-up time of between approximately 29 days and approximately 359 days after said administration of said CAR-T cells, between approximately 29 days and approximately 9 months after said administration of said CAR-T cells, between approximately 29 days and approximately 6 months after said administration of said CAR-T cells, between approximately 29 days and approximately 3 months after said administration of said CAR-T cells, or between approximately 29 days and approximately 2 months after said administration of said CAR-T cells. In some embodiments, said method is effective in maintaining said minimal residual disease (MRD) negative status in said subject assessed in the bone marrow at a second follow-up time of between approximately 180 days and approximately 359 days after said infusion of said CAR-T cells. In some embodiments, said method is effective in maintaining said minimal residual disease (MRD) negative status in said subject assessed in the bone marrow at a second follow-up time of between approximately 360 days and approximately 539 days after said infusion of said CAR-T cells.

In certain embodiments, the efficacy of the method of treatment is assessed by evaluating the proportion of subjects with MRD negative status. In certain embodiments, the efficacy of the method of treatment is assessed by evaluating the proportion of subjects with MRD negative status at a sensitivity level of 10⁻⁶. In certain embodiments, the efficacy of the method of treatment is assessed by evaluating the proportion of subjects with MRD negative status at a sensitivity level of 10⁻⁵. In certain embodiments, the efficacy of the method of treatment is assessed by evaluating the proportion of subjects with MRD negative status at a sensitivity level of 10⁴. In certain embodiments, the efficacy of the method of treatment is assessed by evaluating the proportion of subjects with MRD negative status at a sensitivity level of 10⁻³. In certain embodiments, the efficacy of the method of treatment is assessed by evaluating the proportion of subjects with MRD negative status at a median follow-up time between the administration of the CAR-T cells and approximately 359 days after the administration of the CAR-T cells, between the administration of the CAR-T cells and approximately 9 months after the administration of the CAR-T cells, between the administration of the CAR-T cells and approximately 6 months after the administration of the CAR-T cells, between the administration of the CAR-T cells and approximately 3 months after the administration of the CAR-T cells, between the administration of the CAR-T cells and approximately 2 months after the administration of the CAR-T cells, or between the administration of the CAR-T cells and approximately 29 days after the administration of the CAR-T cells. In some embodiments, said method is effective in obtaining said minimal residual disease (MRD) negative status at a rate of approximately 44% or less at a sensitivity threshold level of 10⁻⁵ at a follow-up time of approximately 12 months after said infusion of said CAR-T cells, a rate of approximately 55% at a sensitivity threshold level of 10⁻⁵ at a follow-up time of approximately 12 months after said administration of said CAR-T cells, a rate of approximately 65% or less at a sensitivity threshold level of 10⁻⁵ at a follow-up time of approximately 12 months after said infusion of said CAR-T cells, a rate of approximately 57% or less at a sensitivity threshold level of 10⁻⁴ at a follow-up time of approximately 18 months after said infusion of said CAR-T cells, a rate of approximately 67% at a sensitivity threshold level of 10⁻⁴ at a follow-up time of approximately 18 months after said infusion of said CAR-T cells, a rate of between approximately 76% or less at a sensitivity threshold level of 10⁻⁴ at a follow-up time of approximately 18 months after said infusion of said CAR-T cells, a rate of approximately 47% or less at a sensitivity threshold level of 10⁻⁵ at a follow-up time of approximately 18 months after said infusion of said CAR-T cells, a rate of approximately 58% at a sensitivity threshold level of 10⁻⁵ at a follow-up time of approximately 18 months after said infusion of said CAR-T cells, a rate of approximately 68% or less at a sensitivity threshold level of 10⁻⁵ at a follow-up time of approximately 18 months after said infusion of said CAR-T cells, a rate of approximately 29% or less at a sensitivity threshold level of 10⁻⁶ at a follow-up time of approximately 18 months after said infusion of said CAR-T cells, a rate of approximately 39% at a sensitivity threshold level of 10⁻⁶ at a follow-up time of approximately 18 months after said infusion of said CAR-T cells or a rate of approximately 50% or less at a sensitivity threshold level of 10⁻⁶ at a follow-up time of approximately 18 months after said infusion of said CAR-T cells. In some embodiments, said method is effective in obtaining said minimal residual disease (MRD) negative status at a rate of between approximately 44% and approximately 65% at a sensitivity threshold level of 10⁻⁵ at a follow-up time of approximately 12 months after said infusion of said CAR-T cells, a rate of between approximately 57% and approximately 76% at a sensitivity threshold level of 10⁻⁴ at a follow-up time of approximately 18 months after said infusion of said CAR-T cells, a rate of between approximately 47% and approximately 68% at a sensitivity threshold level of 10⁻⁵ at a follow-up time of approximately 18 months after said infusion of said CAR-T cells, or a rate of between approximately 29% and approximately 50% at a sensitivity threshold level of 10⁻⁶ at a follow-up time of approximately 18 months after said infusion of said CAR-T cells. In some embodiments, said method is effective in obtaining said minimal residual disease (MRD) negative status at a rate of approximately 55% at a sensitivity threshold level of 10⁻⁵ at a follow-up time of approximately 12 months after said administration of said CAR-T cells, a rate of approximately 67% at a sensitivity threshold level of 10⁻⁴ at a follow-up time of approximately 18 months after said infusion of said CAR-T cells, a rate of approximately 58% at a sensitivity threshold level of 10⁻⁵ at a follow-up time of approximately 18 months after said infusion of said CAR-T cells, or a rate of approximately 39% at a sensitivity threshold level of 10⁻⁶ at a follow-up time of approximately 18 months after said infusion of said CAR-T cells.

In certain embodiments, the efficacy of the method of treatment is assessed by evaluating the proportion of subjects with evaluable bone marrow and MRD negative status. In certain embodiments, the efficacy of the method of treatment is assessed by evaluating the proportion of subjects with evaluable bone marrow and MRD negative status at a sensitivity level of 10⁻⁶. In certain embodiments, the efficacy of the method of treatment is assessed by evaluating the proportion of subjects with evaluable bone marrow and MRD negative status at a sensitivity level of 10⁻⁵. In certain embodiments, the efficacy of the method of treatment is assessed by evaluating the proportion of subjects with evaluable bone marrow and MRD negative status at a sensitivity level of 10⁴. In certain embodiments, the efficacy of the method of treatment is assessed by evaluating the proportion of subjects with evaluable bone marrow and MRD negative status at a sensitivity level of 10⁻³. In certain embodiments, the efficacy of the method of treatment is assessed by evaluating the proportion of subjects with evaluable bone marrow and MRD negative status at a median follow-up time between the administration of the CAR-T cells and approximately 359 days after the administration of the CAR-T cells, between the administration of the CAR-T cells and approximately 9 months after the administration of the CAR-T cells, between the administration of the CAR-T cells and approximately 6 months after the administration of the CAR-T cells, between the administration of the CAR-T cells and approximately 3 months after the administration of the CAR-T cells, between the administration of the CAR-T cells and approximately 2 months after the administration of the CAR-T cells, or between the administration of the CAR-T cells and approximately 29 days after the administration of the CAR-T cells. In some embodiments, said method is effective in obtaining said minimal residual disease (MRD) negative status at a rate of approximately 83% or less in subjects with evaluable samples at a sensitivity threshold level of 10⁻⁵ at a follow-up time of approximately 12 months after said infusion of said CAR-T cells, a rate of approximately 93% in subjects with evaluable samples at a sensitivity threshold level of 10⁻⁵ at a follow-up time of approximately 12 months after said infusion of said CAR-T cells, a rate of approximately 98% or less in subjects with evaluable samples at a sensitivity threshold level of 10⁻⁵ at a follow-up time of approximately 12 months after said infusion of said CAR-T cells, a rate of approximately 82% or less in subjects with evaluable samples at a sensitivity threshold level of 10⁻⁵ at a follow-up time of approximately 18 months after said infusion of said CAR-T cells, a rate of approximately 92% in subjects with evaluable samples at a sensitivity threshold level of 10⁻⁵ at a follow-up time of approximately 18 months after said infusion of said CAR-T cells, or a rate of approximately 97% or less in subjects with evaluable samples at a sensitivity threshold level of 10⁻⁵ at a follow-up time of approximately 18 months after said infusion of said CAR-T cells. In some embodiments, said method is effective in obtaining said minimal residual disease (MRD) negative status at a rate of between approximately 83% and approximately 98% in subjects with evaluable samples at a sensitivity threshold level of 10⁻⁵ at a follow-up time of approximately 12 months after said infusion of said CAR-T cells, or at a rate of between approximately 82% and approximately 97% in subjects with evaluable samples at a sensitivity threshold level of 10⁻⁵ at a follow-up time of approximately 18 months after said infusion of said CAR-T cells. In some embodiments, said method is effective in obtaining said minimal residual disease (MRD) negative status at a rate of approximately 93% in subjects with evaluable samples at a sensitivity threshold level of 10⁻⁵ at a follow-up time of approximately 12 months after said infusion of said CAR-T cells, or at a rate of approximately 92% in subjects with evaluable samples at a sensitivity threshold level of 10⁻⁵ at a follow-up time of approximately 18 months after said infusion of said CAR-T cells.

In some embodiments, said method is effective in obtaining at least one response in the subject after said infusion of said CAR-T cells, wherein said at least one response comprises, in order from better to worse, a stringent complete response, a complete response, a very good partial response, a partial response, or a minimal response.

In some embodiments, said method is effective in obtaining a first response within approximately 27 days or later, approximately 29 days or later, approximately 42 days or later, approximately 89 days or later, or approximately 321 days or later after said infusion of said CAR-T cells. In some embodiments, said method is effective in obtaining a first response before a time of between approximately 27 days and approximately 321 days after said infusion of said CAR-T cells. In some embodiments, said method is effective in obtaining a first response before a time of between approximately 27 days and approximately 89 days after said infusion of said CAR-T cells. In some embodiments, said method is effective in obtaining a first response before approximately 42 days after said infusion of said CAR-T cells. In some embodiments, said method is effective in obtaining a first response before approximately 29 days after said infusion of said CAR-T cells.

In certain embodiments, the efficacy of the method of treatment is assessed by evaluating the proportion of subjects with a stringent complete response. In certain embodiments, the efficacy of the method of treatment is assessed by evaluating the proportion of subjects with a complete response or better. In certain embodiments, the efficacy of the method of treatment is assessed by evaluating the proportion of subjects with a very good partial response or better. In certain embodiments, the efficacy of the method of treatment is assessed by evaluating the proportion of subjects with a partial response or better. In certain embodiments, the efficacy of the method of treatment is assessed by evaluating the proportion of subjects with a minimal response or better.

In some embodiments, said method is effective in obtaining a best response of any one of minimal response, partial response, very good partial response, complete response or stringent complete response, i.e., a best response of minimal response or better. In some embodiments, the rate at which said method is effective in obtaining a best response of minimal response or better is called the clinical benefit rate. In some embodiments, said method is effective in obtaining said best response of minimal response or better at a rate of approximately 91% or less at a follow-up time of approximately 12 months after said infusion of said CAR-T cells, a rate of approximately 97% or less at a follow-up time of approximately 12 months after said infusion of said CAR-T cells, a rate of between approximately 99% or less at a follow-up time of approximately 12 months after said infusion of said CAR-T cells, at a rate of approximately 93% or less at a follow-up time of approximately 18 months after said infusion of said CAR-T cells, a rate of approximately 98% or less at a follow-up time of approximately 18 months after said infusion of said CAR-T cells, or a rate of between approximately 100% or less at a follow-up time of approximately 18 months after said infusion of said CAR-T cells. In some embodiments, said method is effective in obtaining said best response of any one of minimal response, partial response, very good partial response, complete response or stringent complete response at a rate of between approximately 91% and approximately 99% at a follow-up time of approximately 12 months after said infusion of said CAR-T cells or at a rate of between approximately 93% and approximately 100% at a follow-up time of approximately 18 months after said infusion of said CAR-T cells. In some embodiments, said method is effective in obtaining said best response of any one of minimal response, partial response, very good partial response, complete response or stringent complete response at a rate of approximately 97% at a follow-up time of approximately 12 months after said infusion of said CAR-T cells or at a rate of approximately 98% at a follow-up time of approximately 18 months after said infusion of said CAR-T cells.

In some embodiments, the method is effective in obtaining the stringent complete response at a rate of about 40% to about 90%. In some embodiments, the method is effective in obtaining the stringent complete response at a rate of about 50% to about 80%. In some embodiments, the method is effective in obtaining the stringent complete response at a rate of about 58.2%. In some embodiments, the method is effective in obtaining the stringent complete response at a rate of about 68.8%.

In some embodiments, the method is effective in obtaining the complete response at a rate of about 10% to about 20%. In some embodiments, the method is effective in obtaining the complete response at a rate of about 14.9%. In some embodiments, the method is effective in obtaining the complete response at a rate of about 17.6%.

In some embodiments, the method is effective in obtaining a very good partial response.

In some embodiments, the method is effective in obtaining a partial response.

In some embodiments, the method is effective in obtaining a best response of any one of partial response, very good partial response, complete response or stringent complete response, i.e., a best response of partial response or better. In some embodiments, the rate at which said method is effective in obtaining a best response of partial response or better is called the overall survival rate or the overall response rate. In some embodiments, said method is effective in obtaining a best response of partial response or better at a rate of approximately 91% or less at a follow-up time of approximately 12 months after said infusion of said CAR-T cells, a rate of approximately 97% or less at a follow-up time of approximately 12 months after said infusion of said CAR-T cells, a rate of approximately 99% or less at a follow-up time of approximately 12 months after said infusion of said CAR-T cells, a rate of approximately 93% or less at a follow-up time of approximately 18 months after said infusion of said CAR-T cells, a rate of approximately 97% or less at a follow-up time of approximately 18 months after said infusion of said CAR-T cells, or a rate of approximately 100% or less at a follow-up time of approximately 18 months after said infusion of said CAR-T cells. In some embodiments, said method is effective in obtaining said best response of any one of partial response, very good partial response, complete response or stringent complete response at a rate of between approximately 91% and approximately 99% at a follow-up time of approximately 12 months after said infusion of said CAR-T cells or at a rate of between approximately 93% and approximately 100% at a follow-up time of approximately 18 months after said infusion of said CAR-T cells. In some embodiments, said method is effective in obtaining said best response of any one of partial response, very good partial response, complete response or stringent complete response at a rate of approximately 97% at a follow-up time of approximately 12 months after said infusion of said CAR-T cells or at a rate of approximately 97% at a follow-up time of approximately 18 months after said infusion of said CAR-T cells.

In some embodiments, said method is effective in obtaining a best response of any one of very good partial response, complete response or stringent complete response, i.e., a best response of very good partial response or better. In some embodiments, said method is effective in obtaining said best response of very good partial response or better at a rate of approximately 86% or less at a follow-up time of approximately 12 months after said infusion of said CAR-T cells, a rate of approximately 93% or less at a follow-up time of approximately 12 months after said infusion of said CAR-T cells, a rate of approximately 97% or less at a follow-up time of approximately 12 months after said infusion of said CAR-T cells, a rate of approximately 88% or less at a follow-up time of approximately 18 months after said infusion of said CAR-T cells, a rate of approximately 95% or less at a follow-up time of approximately 18 months after said infusion of said CAR-T cells, or a rate of approximately 98% or less at a follow-up time of approximately 18 months after said infusion of said CAR-T cells. In some embodiments, said method is effective in obtaining said best response of any one of very good partial response, complete response or stringent complete response at a rate of between approximately 86% and approximately 97% at a follow-up time of approximately 12 months after said infusion of said CAR-T cells or at a rate of between approximately 88% and approximately 98% at a follow-up time of approximately 18 months after said infusion of said CAR-T cells. In some embodiments, said method is effective in obtaining said best response of any one of very good partial response, complete response or stringent complete response at a rate of approximately 93% at a follow-up time of approximately 12 months after said infusion of said CAR-T cells or at a rate of approximately 95% at a follow-up time of approximately 18 months after said infusion of said CAR-T cells.

In some embodiments, said method is effective in obtaining a best response of complete response or stringent complete response, i.e., a best response of complete response or better. In some embodiments, said method is effective in obtaining said best response of complete response or better at a rate of approximately 57% or less at a follow-up time of approximately 12 months after said infusion of said CAR-T cells, a rate of approximately 67% or less at a follow-up time of approximately 12 months after said infusion of said CAR-T cells, a rate of approximately 76% or less at a follow-up time of approximately 12 months after said infusion of said CAR-T cells, a rate of approximately 73% or less at a follow-up time of approximately 18 months after said infusion of said CAR-T cells, a rate of approximately 83% or less at a follow-up time of approximately 18 months after said infusion of said CAR-T cells, or a rate of approximately 89% or less at a follow-up time of approximately 18 months after said infusion of said CAR-T cells. In some embodiments, said method is effective in obtaining said best response of complete response or stringent complete response at a rate of between approximately 57% and approximately 76% at a follow-up time of approximately 12 months after said infusion of said CAR-T cells or at a rate of between approximately 73% and approximately 89% at a follow-up time of approximately 18 months after said infusion of said CAR-T cells. In some embodiments, said method is effective in obtaining said best response of complete response or stringent complete response at a rate of approximately 67% at a follow-up time of approximately 12 months after said infusion of said CAR-T cells or at a rate of approximately 83% at a follow-up time of approximately 18 months after said infusion of said CAR-T cells.

In some embodiments, said method is effective in obtaining a best response of stringent complete response. In some embodiments, said method is effective in obtaining said best response of stringent complete response at a rate of approximately 57% or less at a follow-up time of approximately 12 months after said infusion of said CAR-T cells, a rate of approximately 67% or less at a follow-up time of approximately 12 months after said infusion of said CAR-T cells, a rate of approximately 76% or less at a follow-up time of approximately 12 months after said infusion of said CAR-T cells, a rate of approximately 73% or less at a follow-up time of approximately 18 months after said infusion of said CAR-T cells, a rate of approximately 83% or less at a follow-up time of approximately 18 months after said infusion of said CAR-T cells, or a rate of approximately 89% or less at a follow-up time of approximately 18 months after said infusion of said CAR-T cells. In some embodiments, said method is effective in obtaining said best response of stringent complete response at a rate of between approximately 57% and approximately 76% at a follow-up time of approximately 12 months after said infusion of said CAR-T cells or at a rate of between approximately 73% and approximately 89% at a follow-up time of approximately 18 months after said infusion of said CAR-T cells. In some embodiments, said method is effective in obtaining said best response of stringent complete response at a rate of approximately 67% at a follow-up time of approximately 12 months after said infusion of said CAR-T cells or at a rate of approximately 83% at a follow-up time of approximately 18 months after said infusion of said CAR-T cells.

In some embodiments, said method is effective in obtaining said best response within approximately 27 days or later, 78 days or later, 153 days or later, 293 days or later, or approximately 534 days or later after said infusion of said CAR-T cells. In some embodiments, said method is effective in obtaining said best response before a time of between approximately 27 days and approximately 534 days after said infusion of said CAR-T cells. In some embodiments, said method is effective in obtaining said best response before a time of between approximately 27 days and approximately 293 days after said infusion of said CAR-T cells. In some embodiments, said method is effective in obtaining said best response before approximately 153 days after said infusion of said CAR-T cells. In some embodiments, said method is effective in obtaining said best response before approximately 78 days after said infusion of said CAR-T cells.

In some embodiments, said method is effective in maintaining a response in the subject at a follow-up time between the time of said first response and approximately 180 days after said infusion of said CAR-T cells, between the time of said first response and approximately 357 days after said infusion of said CAR-T cells, between the time of said first response and approximately 606 days after said infusion of said CAR-T cells, or between the time of said first response and approximately 654 days after said infusion of said CAR-T cells. In some embodiments, said method is effective in maintaining a response at a rate of approximately 77% or less at a follow-up time of approximately 6 months after said infusion of said CAR-T cells, a rate of approximately 85% or less at a follow-up time of approximately 6 months after said infusion of said CAR-T cells, a rate of approximately 91% or less at a follow-up time of approximately 6 months after said infusion of said CAR-T cells, a rate of approximately 63% or less at a follow-up time of approximately 12 months after said infusion of said CAR-T cells, a rate of approximately 74% or less at a follow-up time of approximately 12 months after said infusion of said CAR-T cells, a rate of approximately 81% or less at a follow-up time of approximately 12 months after said infusion of said CAR-T cells, a rate of approximately 56% or less at a follow-up time of approximately 18 months after said infusion of said CAR-T cells, a rate of approximately 67% or less at a follow-up time of approximately 18 months after said infusion of said CAR-T cells, a rate of approximately 75% or less at a follow-up time of approximately 18 months after said infusion of said CAR-T cells, a rate of approximately 52% or less at a follow-up time of approximately 21 months after said infusion of said CAR-T cells, a rate of approximately 63% or less at a follow-up time of approximately 21 months after said infusion of said CAR-T cells, a rate of approximately 72% or less at a follow-up time of approximately 21 months after said infusion of said CAR-T cells, a rate of approximately 48% or less at a follow-up time of approximately 24 months after said infusion of said CAR-T cells, a rate of approximately 60% or less at a follow-up time of approximately 24 months after said infusion of said CAR-T cells, or a rate of approximately 70% or less at a follow-up time of approximately 24 months after said infusion of said CAR-T cells. In some embodiments, said method is effective in maintaining a response at a rate of between approximately 77% and approximately 91% at a follow-up time of approximately 6 months after said infusion of said CAR-T cells, a rate of between approximately 63% and approximately 81% at a follow-up time of approximately 12 months after said infusion of said CAR-T cells, a rate of between approximately 56% and approximately 75% at a follow-up time of approximately 18 months after said infusion of said CAR-T cells, a rate of between approximately 52% and approximately 72% at a follow-up time of approximately 21 months after said infusion of said CAR-T cells, or a rate of between approximately 48% and approximately 70% at a follow-up time of approximately 24 months after said infusion of said CAR-T cells. In some embodiments, said method is effective in maintaining a response at a rate of approximately 85% at a follow-up time of approximately 6 months after said infusion of said CAR-T cells, a rate of approximately 74% at a follow-up time of approximately 12 months after said infusion of said CAR-T cells, a rate of approximately 67% at a follow-up time of approximately 18 months after said infusion of said CAR-T cells, a rate of approximately 63% at a follow-up time of approximately 21 months after said infusion of said CAR-T cells, or a rate of approximately 60% at a follow-up time of approximately 24 months after said infusion of said CAR-T cells.

In some embodiments, wherein said method is effective in obtaining minimal residual disease (MRD) negative status in said subject assessed in the bone marrow at a sensitivity threshold level of 10⁻⁵ between the time of said administration of said CAR-T cells and approximately 3 months after said administration of said CAR-T cells. In some embodiments, said method is effective in obtaining either minimal residual disease (MRD) negative complete response or minimal residual disease (MRD) negative stringent complete response at a rate of approximately 25% or less at a follow-up time of approximately 12 months after said infusion of said CAR-T cells, a rate of approximately 34% or less at a follow-up time of approximately 12 months after said infusion of said CAR-T cells, a rate of approximately 44% or less at a follow-up time of approximately 12 months after said infusion of said CAR-T cells, a rate of approximately 33% or less at a follow-up time of approximately 18 months after said infusion of said CAR-T cells, a rate of approximately 43% or less at a follow-up time of approximately 18 months after said infusion of said CAR-T cells, or a rate of approximately 54% or less at a follow-up time of approximately 18 months after said infusion of said CAR-T cells. In some embodiments, said method is effective in obtaining either minimal residual disease (MRD) negative complete response or minimal residual disease (MRD) negative stringent complete response at a rate of between approximately 25% and approximately 44% at a follow-up time of approximately 12 months after said infusion of said CAR-T cells or at a rate of between approximately 33% and approximately 54% at a follow-up time of approximately 18 months after said infusion of said CAR-T cells. In some embodiments, said method is effective in obtaining either minimal residual disease (MRD) negative complete response or minimal residual disease (MRD) negative stringent complete response at a rate of approximately 34% at a follow-up time of approximately 12 months after said infusion of said CAR-T cells or at a rate of approximately 43% at a follow-up time of approximately 18 months after said infusion of said CAR-T cells.

In some embodiments, said method is effective in obtaining progression-free survival of the subject. In some embodiments, said method is effective in obtaining said progression-free survival of the subject at a time between said infusion of said CAR-T cells and approximately 209 days after said infusion of said CAR-T cells, between said infusion of said CAR-T cells and approximately 386 days after said infusion of said CAR-T cells, between said infusion of said CAR-T cells and approximately 632 days after said infusion of said CAR-T cells, or between said infusion of said CAR-T cells and approximately 684 days after said infusion of said CAR-T cells. In some embodiments, said method is effective in obtaining said progression-free survival at a rate of approximately 79% or more at a follow-up time of approximately 6 months after said infusion of said CAR-T cells, a rate of approximately 88% or more at a follow-up time of approximately 6 months after said infusion of said CAR-T cells, a rate of approximately 93% or more at a follow-up time of approximately 6 months after said infusion of said CAR-T cells, a rate of approximately 67% or more at a follow-up time of approximately 12 months after said infusion of said CAR-T cells, a rate of approximately 76% or more at a follow-up time of approximately 12 months after said infusion of said CAR-T cells, a rate of approximately 84% or more at a follow-up time of approximately 12 months after said infusion of said CAR-T cells, a rate of approximately 57% or more at a follow-up time of approximately 18 months after said infusion of said CAR-T cells, a rate of approximately 67% or more at a follow-up time of approximately 18 months after said infusion of said CAR-T cells, a rate of approximately 75% or more at a follow-up time of approximately 18 months after said infusion of said CAR-T cells, a rate of approximately 57% or more at a follow-up time of approximately 21 months after said infusion of said CAR-T cells, a rate of approximately 67% or more at a follow-up time of approximately 21 months after said infusion of said CAR-T cells, a rate of approximately 75% or more at a follow-up time of approximately 21 months after said infusion of said CAR-T cells, a rate of approximately 49% or more at a follow-up time of approximately 24 months after said infusion of said CAR-T cells, a rate of approximately 61% or more at a follow-up time of approximately 24 months after said infusion of said CAR-T cells, or a rate of approximately 70% or more at a follow-up time of approximately 24 months after said infusion of said CAR-T cells. In some embodiments, said method is effective in obtaining said progression-free survival at a rate of between approximately 79% and approximately 93% at a follow-up time of approximately 6 months after said infusion of said CAR-T cells, a rate of between approximately 67% and approximately 84% at a follow-up time of approximately 12 months after said infusion of said CAR-T cells, a rate of between approximately 57% and approximately 75% at a follow-up time of approximately 18 months after said infusion of said CAR-T cells, a rate of between approximately 57% and approximately 75% at a follow-up time of approximately 21 months after said infusion of said CAR-T cells, or a rate of between approximately 49% and approximately 70% at a follow-up time of approximately 24 months after said infusion of said CAR-T cells. In some embodiments, said method is effective in obtaining said progression-free survival at a rate of approximately 88% at a follow-up time of approximately 6 months after said infusion of said CAR-T cells, a rate of approximately 76% at a follow-up time of approximately 12 months after said infusion of said CAR-T cells, a rate of approximately 67% at a follow-up time of approximately 18 months after said infusion of said CAR-T cells, a rate of approximately 67% at a follow-up time of approximately 21 months after said infusion of said CAR-T cells, or a rate of approximately 61% at a follow-up time of approximately 24 months after said infusion of said CAR-T cells.

In some embodiments, said method further comprises treating said subject for cytokine release syndrome more than approximately 1 day after said infusion of said CAR-T cells. In some embodiments, said method is effective in obtaining a rate of recovery from said cytokine release syndrome of between approximately 1% and approximately 99% at a time of approximately 1, 3, 4, 6, 16 or 97 days after first observance of said cytokine release syndrome.

In some embodiments, said method further comprises treating said subject for immune effector cell-associated neurotoxicity more than approximately 3 days after said infusion of said CAR-T cells. In some embodiments, said method is effective in obtaining a rate of recovery from said immune effector cell-associated neurotoxicity of between approximately 1% and approximately 17% at a time of approximately 1, 4, 5, 8, 12 or 16 days after first observance of said immune effector cell-associated neurotoxicity.

In certain embodiments, the method of treatment is effective in obtaining in the subject a reduction in tumor burden. In certain embodiments, the method of treatment is effective in obtaining a reduction in tumor burden in greater than 90% of the subjects. In certain embodiments, the method of treatment is effective in obtaining a reduction in tumor burden in greater than 91% of the subjects. In certain embodiments, the method of treatment is effective in obtaining a reduction in tumor burden in greater than 92% of the subjects. In certain embodiments, the method of treatment is effective in obtaining a reduction in tumor burden in greater than 93% of the subjects. In certain embodiments, the method of treatment is effective in obtaining a reduction in tumor burden in greater than 94% of the subjects. In certain embodiments, the method of treatment is effective in obtaining a reduction in tumor burden in greater than 95% of the subjects. In certain embodiments, the method of treatment is effective in obtaining a reduction in tumor burden in greater than 96% of the subjects. In certain embodiments, the method of treatment is effective in obtaining a reduction in tumor burden in greater than 97% of the subjects. In certain embodiments, the method of treatment is effective in obtaining a reduction in tumor burden in greater than 98% of the subjects. In certain embodiments, the method of treatment is effective in obtaining a reduction in tumor burden in greater than 99% of the subjects. In some embodiments, the method of treatment is effective in obtaining a reduction in tumor burden in 100% of the subjects.

In certain embodiments, the method of treatment is effective in obtaining in the subject minimal residual disease (MRD) negative status or maintaining said minimal residual disease (MRD) status. In certain embodiments, the method of treatment is effective in obtaining in the subject minimal residual disease (MRD) negative status. In certain embodiments, the method of treatment is effective in obtaining in the subject a minimal residual disease (MRD) negative status at a sensitivity level of 10⁻⁶. In certain embodiments, the method of treatment is effective in obtaining in the subject minimal residual disease (MRD) negative status at a sensitivity level of 10⁻⁵. In certain embodiments, the method of treatment is effective in obtaining in the subject minimal residual disease (MRD) negative status at a sensitivity level of 10⁻⁴. In certain embodiments, the method of treatment is effective in obtaining in the subject minimal residual disease (MRD) negative status at a sensitivity level of 10⁻³. In certain embodiments, the method of treatment is effective in obtaining MRD negative status when assessed in the bone marrow. In certain embodiments, the method of treatment is effective in maintaining the MRD negative status when assessed using a bone marrow sample that is evaluable. In certain embodiments, the method of treatment is effective in obtaining MRD negative status when assessed using bone marrow DNA. In certain embodiments, the method of treatment is effective in obtaining MRD negative status when assessed at a follow-up time of greater than or equal to 28 days following the administration of CAR-T cells. In certain embodiments, the method of treatment is effective in obtaining MRD negative status when assessed at a follow-up time of greater than or equal to 1 month following the administration of CAR-T cells. In certain embodiments, the method of treatment is effective in obtaining MRD negative status when assessed at a follow-up time of greater than or equal to 3 months following the administration of CAR-T cells. In certain embodiments, the method of treatment is effective in obtaining MRD negative status when assessed at a follow-up time of greater than or equal to 6 months following the administration of CAR-T cells. In certain embodiments, the method of treatment is effective in obtaining MRD negative status when assessed at a follow-up time of greater than or equal to 9 months following the administration of CAR-T cells. In certain embodiments, the method of treatment is effective in obtaining MRD negative status when assessed at a follow-up time of greater than or equal to 12 months following the administration of CAR-T cells.

In certain embodiments, the method of treatment is effective in maintaining in the subject a first obtained minimal residual disease (MRD) negative status. In certain embodiments, the method of treatment is effective in maintaining MRD negative status at a sensitivity level of 10⁻⁵. In certain embodiments, the method of treatment is effective in obtaining in the subject minimal residual disease (MRD) negative status at a sensitivity level of 10⁻⁶. In certain embodiments, the method of treatment is effective in maintaining MRD negative status at a sensitivity level of 10⁻⁴. In certain embodiments, the method of treatment is effective in maintaining MRD negative status at a sensitivity level of 10⁻³. In certain embodiments, the method of treatment is effective in maintaining the MRD negative status when assessed using a bone marrow sample. In certain embodiments, the method of treatment is effective in maintaining the MRD negative status when assessed using a bone marrow sample that is evaluable. In certain embodiments, the method of treatment is effective in maintaining MRD negative status is maintained when assessed using bone marrow DNA. In certain embodiments, the method of treatment is effective in maintaining MRD negative status when assessed at a follow-up time of greater than or equal to 1 month following the administration of CAR-T cells. In certain embodiments, the method of treatment is effective in maintaining MRD negative status when assessed at a follow-up time of greater than or equal to 3 months following the administration of CAR-T cells. In certain embodiments, the method of treatment is effective in maintaining MRD negative status when assessed at a follow-up time of greater than or equal to 6 months following the administration of CAR-T cells. In certain embodiments, the method of treatment is effective in maintaining MRD negative status when assessed at a follow-up time of greater than or equal to 9 months following the administration of CAR-T cells. In certain embodiments, the method of treatment is effective in maintaining MRD negative status when assessed at a follow-up time of greater than or equal to 12 months following the administration of CAR-T cells.

In certain embodiments, the efficacy of the method of treatment is assessed by evaluating the proportion of subjects with MRD negative status. In certain embodiments, the efficacy of the method of treatment is assessed by evaluating the proportion of subjects with MRD negative status at a sensitivity level of 10⁻⁶. In certain embodiments, the efficacy of the method of treatment is assessed by evaluating the proportion of subjects with MRD negative status at a sensitivity level of 10⁻⁵. In certain embodiments, the efficacy of the method of treatment is assessed by evaluating the proportion of subjects with MRD negative status at a sensitivity level of 10⁻⁴. In certain embodiments, the efficacy of the method of treatment is assessed by evaluating the proportion of subjects with MRD negative status at a sensitivity level of 10⁻³. In certain embodiments, the efficacy of the method of treatment is assessed by evaluating the proportion of subjects with MRD negative status at a median follow-up time of greater than or equal to 28 days following the administration of CAR-T cells. In certain embodiments, the efficacy of the method of treatment is assessed by evaluating the proportion of subjects with MRD negative status at a median follow-up time of greater than or equal to 1 month following the administration of CAR-T cells. In certain embodiments, the efficacy of the method of treatment is assessed by evaluating the proportion of subjects with MRD negative status at a median follow-up time of greater than or equal to 3 months following the administration of CAR-T cells. In certain embodiments, the efficacy of the method of treatment is assessed by evaluating the proportion of subjects with MRD negative status at a median follow-up time of greater than or equal to 6 months following the administration of CAR-T cells. In certain embodiments, the efficacy of the method of treatment is assessed by evaluating the proportion of subjects with MRD negative status at a median follow-up time of greater than or equal to 9 months following the administration of CAR-T cells. In certain embodiments, the efficacy of the method of treatment is assessed by evaluating the proportion of subjects with MRD negative status at a median follow-up time of greater than or equal to 12 months following the administration of CAR-T cells.

In certain embodiments, the efficacy of the method of treatment is assessed by evaluating the proportion of subjects with evaluable bone marrow and MRD negative status. In certain embodiments, the efficacy of the method of treatment is assessed by evaluating the proportion of subjects with evaluable bone marrow and MRD negative status at a sensitivity level of 10⁻⁶. In certain embodiments, the efficacy of the method of treatment is assessed by evaluating the proportion of subjects with evaluable bone marrow and MRD negative status at a sensitivity level of 10⁻⁵. In certain embodiments, the efficacy of the method of treatment is assessed by evaluating the proportion of subjects with evaluable bone marrow and MRD negative status at a sensitivity level of 10⁻⁴. In certain embodiments, the efficacy of the method of treatment is assessed by evaluating the proportion of subjects with evaluable bone marrow and MRD negative status at a sensitivity level of 10⁻³. In certain embodiments, the efficacy of the method of treatment is assessed by evaluating the proportion of subjects with evaluable bone marrow and MRD negative status at a median follow-up time of greater than or equal to 28 days following the administration of CAR-T cells. In certain embodiments, the efficacy of the method of treatment is assessed by evaluating the proportion of subjects with evaluable bone marrow and MRD negative status at a median follow-up time of greater than or equal to 1 month following the administration of CAR-T cells. In certain embodiments, the efficacy of the method of treatment is assessed by evaluating the proportion of subjects with evaluable bone marrow and MRD negative status at a median follow-up time of greater than or equal to 3 months following the administration of CAR-T cells. In certain embodiments, the efficacy of the method of treatment is assessed by evaluating the proportion of subjects with evaluable bone marrow and MRD negative status at a median follow-up time of greater than or equal to 6 months following the administration of CAR-T cells. In certain embodiments, the efficacy of the method of treatment is assessed by evaluating the proportion of subjects with evaluable bone marrow and MRD negative status at a median follow-up time of greater than or equal to 9 months following the administration of CAR-T cells. In certain embodiments, the efficacy of the method of treatment is assessed by evaluating the proportion of subjects with evaluable bone marrow and MRD negative status at a median follow-up time of greater than or equal to 12 months following the administration of CAR-T cells.

In certain embodiments, the efficacy of the method of treatment is assessed by evaluating the proportion of subjects with a stringent complete response. In certain embodiments, the efficacy of the method of treatment is assessed by evaluating the proportion of subjects with a complete response or better. In certain embodiments, the efficacy of the method of treatment is assessed by evaluating the proportion of subjects with a very good partial response or better. In certain embodiments, the efficacy of the method of treatment is assessed by evaluating the proportion of subjects with a partial response or better. In certain embodiments, the efficacy of the method of treatment is assessed using an overall response rate. In some embodiments, the overall response rate is the proportion of subjects with a partial response or better.

In certain embodiments, the method is effective in obtaining a minimal residual disease (MRD) negativity rate of greater than 39% at a sensitivity threshold level of 10⁻⁵. In certain embodiments, the method is effective in obtaining a minimal residual disease (MRD) negativity rate of greater than 44% at a sensitivity threshold level of 10⁻⁵. In certain embodiments, the method is effective in obtaining a minimal residual disease (MRD) negativity rate of greater than 49% at a sensitivity threshold level of 10⁻⁵. In certain embodiments, the method is effective in obtaining a minimal residual disease (MRD) negativity rate of greater than 54% at a sensitivity threshold level of 10⁻⁵. In certain embodiments, the method is effective in obtaining a minimal residual disease (MRD) negativity rate of greater than 59% at a sensitivity threshold level of 10⁻⁵. In certain embodiments, the method is effective in obtaining a minimal residual disease (MRD) negativity rate of greater than 64% at a sensitivity threshold level of 10⁻⁵. In certain embodiments, the method is effective in obtaining a minimal residual disease (MRD) negativity rate of greater than 69% at a sensitivity threshold level of 10⁻⁵. In certain embodiments, the method is effective in obtaining a minimal residual disease (MRD) negativity rate of greater than 74% at a sensitivity threshold level of 10⁻⁵. In certain embodiments, the method is effective in obtaining a minimal residual disease (MRD) negativity rate of greater than 70% at a sensitivity threshold level of 10⁻⁵. In certain embodiments, the method is effective in obtaining a minimal residual disease (MRD) negativity rate of greater than 75% at a sensitivity threshold level of 10⁻⁵ in evaluable bone marrow. In certain embodiments, the method is effective in obtaining a minimal residual disease (MRD) negativity rate of greater than 80% at a sensitivity threshold level of 10⁻⁵ in evaluable bone marrow. In certain embodiments, the method is effective in obtaining a minimal residual disease (MRD) negativity rate of greater than 85% at a sensitivity threshold level of 10⁻⁵ in evaluable bone marrow. In certain embodiments, the method is effective in obtaining a minimal residual disease (MRD) negativity rate of greater than 90% at a sensitivity threshold level of 10⁻⁵ in evaluable bone marrow. In certain embodiments, the method is effective in obtaining a minimal residual disease (MRD) negativity rate of greater than 95% at a sensitivity threshold level of 10⁻⁵ in evaluable bone marrow. In certain embodiments, the method is effective in obtaining a minimal residual disease (MRD) negativity rate of 100% at a sensitivity threshold level of 10⁻⁵ in evaluable bone marrow.

In certain embodiments, the method of treatment is effective in obtaining an overall response rate of greater than 75%. In certain embodiments, the method of treatment is effective in obtaining an overall response rate of greater than 80%. In certain embodiments, the method of treatment is effective in obtaining an overall response rate of greater than 85%. In certain embodiments, the method of treatment is effective in obtaining an overall response rate of greater than 90%. In certain embodiments, the method of treatment is effective in obtaining an overall response rate of greater than 91%. In certain embodiments, the method is effective in obtaining an overall response rate of greater than 93%. In certain embodiments, the method is effective in obtaining an overall response rate of greater than 95%. In certain embodiments, the method is effective in obtaining an overall response rate of greater than 97%. In certain embodiments, the method is effective in obtaining an overall response rate of greater than 99%. In some embodiments, the method is effective in obtaining an overall response rate of 100%. In certain embodiments, the method of treatment is effective in obtaining an overall response rate of about 84.6%. In certain embodiments, the method of treatment is effective in obtaining an overall response rate of about 99.4%. In certain embodiments, the overall response rate is assessed at a median follow-up time of at least 6 months following said infusion of said CAR-T cells. In certain embodiments, the overall response rate is assessed at a median follow-up time of at least 12 months following said infusion of said CAR-T cells.

In certain embodiments, more than 70% of subjects are responding to the method of treatment at 9 months after the administration of CAR-T cells. In certain embodiments, more than 72% of subjects are responding to the method of treatment at 9 months after the administration of CAR-T cells. In certain embodiments, more than 74% of subjects are responding to the method of treatment at 9 months after the administration of CAR-T cells. In certain embodiments, more than 76% of subjects are responding to the method of treatment at 9 months after the administration of CAR-T cells. In certain embodiments, more than 78% of subjects are responding to the method of treatment at 9 months after the administration of CAR-T cells. In certain embodiments, more than 80% of subjects are responding to the method of treatment at 9 months after the administration of CAR-T cells. In certain embodiments, more than 82% of subjects are responding to the method of treatment at 9 months after the administration of CAR-T cells. In certain embodiments, more than 84% of subjects are responding to the method of treatment at 9 months after the administration of CAR-T cells. In certain embodiments, more than 86% of subjects are responding to the method of treatment at 9 months after the administration of CAR-T cells.

In certain embodiments, more than 54% of responding subjects are responding to the method of treatment at 12 months after the administration of CAR-T cells. In certain embodiments, more than 58% of responding subjects are responding to the method of treatment at 12 months after the administration of CAR-T cells. In certain embodiments, more than 62% of responding subjects are responding to the method of treatment at 12 months after the administration of CAR-T cells. In certain embodiments, more than 66% of responding subjects are responding to the method of treatment at 12 months after the administration of CAR-T cells. In certain embodiments, more than 70% of responding subjects are responding to the method of treatment at 12 months after the administration of CAR-T cells. In certain embodiments, more than 74% of responding subjects are responding to the method of treatment at 12 months after the administration of CAR-T cells. In certain embodiments, more than 78% of responding subjects are responding to the method of treatment at 12 months after the administration of CAR-T cells.

In certain embodiments, the method of treatment is effective in obtaining a duration of response greater than 9 months, 10 months, 11 months, 12 months, 13 months, 14 months, 15 months, 16 months, 17 months, 18 months, 19 months, 20 months, 21 months, 22 months, 23 months, 24 months, or longer. In certain embodiments, the method of treatment is effective in obtaining a duration of response greater than 12.4 months. In certain embodiments, the method of treatment is effective in obtaining a duration of response greater than 15.9 months.

In certain embodiments, the method of treatment is effective in obtaining a median duration of response greater than 9 months, 10 months, 11 months, 12 months, 13 months, 14 months, 15 months, 16 months, 17 months, 18 months, 19 months, 20 months, 21 months, 22 months, 23 months, 24 months, or longer. In certain embodiments, the method of treatment is effective in obtaining a median duration of response greater than 12.4 months. In certain embodiments, the method of treatment is effective in obtaining a median duration of response greater than 15.9 months.

In certain embodiments, the method of treatment is effective in obtaining a complete response or better in greater than 60% of the subjects. In certain embodiments, the method of treatment is effective in obtaining a complete response or better in greater than 61% of the subjects. In certain embodiments, the method of treatment is effective in obtaining a complete response or better in greater than 62% of the subjects. In certain embodiments, the method of treatment is effective in obtaining a complete response or better in greater than 63% of the subjects. In certain embodiments, the method of treatment is effective in obtaining a complete response or better in greater than 64% of the subjects. In certain embodiments, the method of treatment is effective in obtaining a complete response or better in greater than 65% of the subjects. In certain embodiments, the method of treatment is effective in obtaining a complete response or better in greater than 66% of the subjects. In certain embodiments, the method of treatment is effective in obtaining a complete response or better in greater than 67% of the subjects. In certain embodiments, the complete response or better is assessed less than 1 month after the administration of CAR-T cells. In certain embodiments, the complete response or better is assessed less than 3 months after the administration of CAR-T cells. In certain embodiments, the complete response or better is assessed less than 6 months after the administration of CAR-T cells. In certain embodiments, the complete response or better is assessed less than 9 months after the administration of CAR-T cells. In certain embodiments, the complete response or better is assessed less than 12 months after the administration of CAR-T cells. In certain embodiments, the complete response or better is assessed less than 15 months after the administration of CAR-T cells. In certain embodiments, the complete response or better is assessed more than 15 months the administration of CAR-T cells.

In certain embodiments, the method of treatment is effective in obtaining a very good partial response or better in greater than 80% of the subjects. In certain embodiments, the method of treatment is effective in obtaining a very good partial response or better in greater than 85% of the subjects. In certain embodiments, the method of treatment is effective in obtaining a very good partial response or better in greater than 86% of the subjects. In certain embodiments, the method of treatment is effective in obtaining a very good partial response or better in greater than 87% of the subjects. In certain embodiments, the method of treatment is effective in obtaining a very good partial response or better in greater than 88% of the subjects. In certain embodiments, the method of treatment is effective in obtaining a very good partial response or better in greater than 89% of the subjects. In certain embodiments, the method of treatment is effective in obtaining a very good partial response or better in greater than 90% of the subjects. In certain embodiments, the method of treatment is effective in obtaining a very good partial response or better in greater than 91% of the subjects. In certain embodiments, the method of treatment is effective in obtaining a very good partial response or better in greater than 92% of the subjects. In certain embodiments, the very good partial response or better is assessed less than 1 month after the administration of CAR-T cells. In certain embodiments, the very good partial response or better is assessed less than 3 months after the administration of CAR-T cells. In certain embodiments, the very good partial response or better is assessed less than 6 months after the administration of CAR-T cells. In certain embodiments, the very good partial response or better is assessed less than 9 months after the administration of CAR-T cells. In certain embodiments, the very good partial response or better is assessed less than 12 months after the administration of CAR-T cells. In certain embodiments, the very good partial response or better is assessed less than 15 months after the administration of CAR-T cells. In certain embodiments, the very good partial response or better is assessed more than 15 months the administration of CAR-T cells.

In certain embodiments, the method of treatment is effective in obtaining a median time to first response of less than 1.15 months. In certain embodiments, the method of treatment is effective in obtaining a median time to first response of less than 1.10 months. In certain embodiments, the method of treatment is effective in obtaining a median time to first response of less than 1.05 months. In certain embodiments, the method of treatment is effective in obtaining a median time to first response of less than 1.00 months. In certain embodiments, the method of treatment is effective in obtaining a median time to first response of less than 0.95 months.

In certain embodiments, the method of treatment is effective in obtaining a median time to best response of less than 2.96 months. In certain embodiments, the method of treatment is effective in obtaining a median time to best response of less than 2.86 months. In certain embodiments, the method of treatment is effective in obtaining a median time to best response of less than 2.76 months. In certain embodiments, the method of treatment is effective in obtaining a median time to best response of less than 2.66 months. In certain embodiments, the method of treatment is effective in obtaining a median time to best response of less than 2.56 months.

In certain embodiments, the method is effective in obtaining an overall survival rate of greater than 80% at 9 months after the administration of CAR-T cells. In certain embodiments, the method is effective in obtaining an overall survival rate of greater than 82% at 9 months after the administration of CAR-T cells. In certain embodiments, the method is effective in obtaining an overall survival rate of greater than 85% at 9 months after the administration of CAR-T cells. In certain embodiments, the method is effective in obtaining an overall survival rate of greater than 87% at 9 months after the administration of CAR-T cells. In certain embodiments, the method is effective in obtaining an overall survival rate of greater than 90% at 9 months after the administration of CAR-T cells. In certain embodiments, the method is effective in obtaining an overall survival rate of greater than 92% at 9 months after the administration of CAR-T cells. In certain embodiments, the method is effective in obtaining an overall survival rate of greater than 95% at 9 months after the administration of CAR-T cells.

In certain embodiments, the method is effective in obtaining an overall survival rate of greater than 80% at 12 months after the administration of CAR-T cells. In certain embodiments, the method is effective in obtaining an overall survival rate of greater than 83% at 12 months after the administration of CAR-T cells. In certain embodiments, the method is effective in obtaining an overall survival rate of greater than 86% at 12 months after the administration of CAR-T cells. In certain embodiments, the method is effective in obtaining an overall survival rate of greater than 89% at 12 months after the administration of CAR-T cells. In certain embodiments, the method is effective in obtaining an overall survival rate of greater than 92% at 12 months after the administration of CAR-T cells. In certain embodiments, the method is effective in obtaining an overall survival rate of greater than 93% at 12 months after the administration of CAR-T cells.

In certain embodiments, the method is effective in obtaining a progression free survival rate of greater than 70% at 9 months after the administration of CAR-T cells. In certain embodiments, the method is effective in obtaining a progression free survival rate of greater than 72% at 9 months after the administration of CAR-T cells. In certain embodiments, the method is effective in obtaining a progression free survival rate of greater than 75% at 9 months after the administration of CAR-T cells. In certain embodiments, the method is effective in obtaining a progression free survival rate of greater than 77% at 9 months after the administration of CAR-T cells. In certain embodiments, the method is effective in obtaining a progression free survival rate of greater than 80% at 9 months after the administration of CAR-T cells. In certain embodiments, the method is effective in obtaining a progression free survival rate of greater than 82% at 9 months after the administration of CAR-T cells. In certain embodiments, the method is effective in obtaining a progression free survival rate of greater than 85% at 9 months after the administration of CAR-T cells. In certain embodiments, the method is effective in obtaining a progression free survival rate of greater than or equal to 87% at 9 months after the administration of CAR-T cells.

In certain embodiments, the method is effective in obtaining a progression free survival rate of greater than 66% at 12 months after the administration of CAR-T cells. In certain embodiments, the method is effective in obtaining a progression free survival rate of greater than 69% at 12 months after the administration of CAR-T cells. In certain embodiments, the method is effective in obtaining a progression free survival rate of greater than 72% at 12 months after the administration of CAR-T cells. In certain embodiments, the method is effective in obtaining a progression free survival rate of greater than 76% at 12 months after the administration of CAR-T cells. In certain embodiments, the method is effective in obtaining a progression free survival rate of greater than 80% at 12 months after the administration of CAR-T cells. In certain embodiments, the method is effective in obtaining a progression free survival rate of greater than 84% at 12 months after the administration of the CAR-T cells.

In certain embodiments, the method of treatment is effective in obtaining that greater than 86% of subjects recover from cytokine release syndrome. In certain embodiments, the method of treatment is effective in obtaining that greater than 88% of subjects recover from cytokine release syndrome. In certain embodiments, the method of treatment is effective in obtaining that greater than 90% of subjects recover from cytokine release syndrome. In certain embodiments, the method of treatment is effective in obtaining that greater than 92% of subjects recover from cytokine release syndrome. In certain embodiments, the method of treatment is effective in obtaining that greater than 94% of subjects recover from cytokine release syndrome. In certain embodiments, the method of treatment is effective in obtaining that greater than 96% of subjects recover from cytokine release syndrome. In certain embodiments, the method of treatment is effective in obtaining that greater than 98% of subjects recover from cytokine release syndrome. In certain embodiments, the method of treatment is effective in obtaining that greater than 99% of subjects recover from cytokine release syndrome. In certain embodiments, the method of treatment is effective in obtaining that 100% of subjects recover from cytokine release syndrome.

In certain embodiments, the method of treatment is effective in obtaining that greater than 90% of subjects recover from immune effector cell-associated neurotoxicity, if any. In certain embodiments, the method of treatment is effective in obtaining that greater than 92% of subjects recover from immune effector cell-associated neurotoxicity, if any. In certain embodiments, the method of treatment is effective in obtaining that greater than 94% of subjects recover from immune effector cell-associated neurotoxicity, if any. In certain embodiments, the method of treatment is effective in obtaining that greater than 96% of subjects recover from immune effector cell-associated neurotoxicity, if any. In certain embodiments, the method of treatment is effective in obtaining that greater than 98% of subjects recover from immune effector cell-associated neurotoxicity, if any. In certain embodiments, the method of treatment is effective in obtaining that 100% of subjects recover from immune effector cell-associated neurotoxicity, if any.

In some embodiments, the method further comprises diagnosing said subject for cytopenias. In some embodiments, the cytopenias comprise one or more of, or all of, lymphopenia, neutropenia, and thrombocytopenia. Without being bound by theory, a Grade 3 or Grade 4 but not a Grade 2 or lower lymphopenia is characterized by to a lymphocyte count less than 0.5×10⁹ cells per liter of a subject's blood sample, a Grade 3 or Grade 4 but not a Grade 2 or lower neutropenia is characterized by a neutrophil count less than 1000 cells per microliter of a subject's blood sample, and a Grade 3 or Grade 4 but not a Grade 2 or lower thrombocytopenia is characterized by a platelet count less than 50,000 cells per microliter of a subject's blood sample. In some embodiments, greater than 75% of subjects with Grade 3 or Grade 4 lymphopenia following CAR-T cell administration recover to Grade 2 or lower lymphopenia 60 days following CAR-T cell administration. In some embodiments, greater than 80% of subjects with Grade 3 or Grade 4 lymphopenia following CAR-T cell administration recover to Grade 2 or lower lymphopenia 60 days following CAR-T cell administration. In some embodiments, greater than 85% of subjects with Grade 3 or Grade 4 lymphopenia following CAR-T cell administration recover to Grade 2 or lower lymphopenia 60 days following CAR-T cell administration. In some embodiments, greater than 90% of subjects with Grade 3 or Grade 4 lymphopenia following CAR-T cell administration recover to Grade 2 or lower lymphopenia 60 days following CAR-T cell administration. In some embodiments, greater than 70% of subjects with Grade 3 or Grade 4 neutropenia following CAR-T cell administration recover to Grade 2 or lower neutropenia 60 days following CAR-T cell administration. In some embodiments, greater than 75% of subjects with Grade 3 or Grade 4 neutropenia following CAR-T cell administration recover to Grade 2 or lower neutropenia 60 days following CAR-T cell administration. In some embodiments, greater than 80% of subjects with Grade 3 or Grade 4 neutropenia following CAR-T cell administration recover to Grade 2 or lower neutropenia 60 days following CAR-T cell administration. In some embodiments, greater than 85% of subjects with Grade 3 or Grade 4 neutropenia following CAR-T cell administration recover to Grade 2 or lower neutropenia 60 days following CAR-T cell administration. In some embodiments, greater than 30% of subjects with Grade 3 or Grade 4 thrombocytopenia following CAR-T cell administration recover to Grade 2 or lower thrombocytopenia 60 days following CAR-T cell administration. In some embodiments, greater than 34% of subjects with Grade 3 or Grade 4 thrombocytopenia following CAR-T cell administration recover to Grade 2 or lower thrombocytopenia 60 days following CAR-T cell administration. In some embodiments, greater than 38% of subjects with Grade 3 or Grade 4 thrombocytopenia following CAR-T cell administration recover to Grade 2 or lower thrombocytopenia 60 days following CAR-T cell administration. In some embodiments, greater than 42% of subjects with Grade 3 or Grade 4 thrombocytopenia following CAR-T cell administration recover to Grade 2 or lower thrombocytopenia 60 days following CAR-T cell administration.

In certain embodiments, the subject is re-treated by administration via a second intravenous infusion of a second dose of CAR-T cells. In certain embodiments, the re-treatment dose comprises 1.0×10⁵ to 5.0×10⁶ of CAR-T cells per kilogram of the mass of the subject. In certain embodiments, the re-treatment dose comprises approximately 0.75×10⁵ of CAR-T cells per kilogram of the mass of the subject. In certain embodiments, the subject is re-treated upon exhibiting progressive disease after a best response of minimal response or better following the first infusion of CAR-T cells. In certain embodiments, the time between the first infusion of CAR-T cells and the detection of the progressive disease comprises at least six months.

### Kits and Articles of Manufacture

Any of the compositions described herein may be comprised in a kit. In some embodiments, engineered immortalized CAR-T cells are provided in the kit, which also may include reagents suitable for expanding the cells, such as media.

In a non-limiting example, a chimeric receptor expression construct, one or more reagents to generate a chimeric receptor expression construct, cells for transfection of the expression construct, and/or one or more instruments to obtain immortalized T cells for transfection of the expression construct (such an instrument may be a syringe, pipette, forceps, and/or any such medically approved apparatus).

In some embodiments, the kit comprises reagents or apparatuses for electroporation of cells.

In some embodiments, the kit comprises artificial antigen presenting cells.

The kits may comprise one or more suitably aliquoted compositions of the present disclosure or reagents to generate compositions of the disclosure. The components of the kits may be packaged either in aqueous media or in lyophilized form. The container means of the kits may include at least one vial, test tube, flask, bottle, syringe, or other container means, into which a component may be placed, and preferably, suitably aliquoted. Where there is more than one component in the kit, the kit also will generally contain a second, third, or other additional container into which the additional components may be separately placed. However, various combinations of components may be comprised in a vial. The kits of the present disclosure also will typically include a means for containing the chimeric receptor construct and any other reagent containers in close confinement for commercial sale. Such containers may include injection or blow molded plastic containers into which the desired vials are retained, for example.

The exemplary embodiments below are intended to be purely exemplary of the disclosure and should therefore not be considered to limit the disclosure in any way.

### EXEMPLARY EMBODIMENTS

1. A method of treating a subject, comprising administering to the subject a dose of T cells comprising a chimeric antigen receptor (CAR) comprising:
(a) an extracellular antigen binding domain capable of specifically binding to an epitope of B-cell maturation antigen (BCMA),
(b) a transmembrane domain, and
(c) an intracellular signaling domain,
wherein the subject has multiple myeloma, has received one to three prior lines of therapies, including a therapy with an immunomodulatory drug (IMiD), and is refractory to the IMiD.

2. The method of embodiment 1, wherein the subject has a high-risk feature, and wherein optionally the high-risk feature is a cytogenetic abnormality, International Staging System (ISS) stage III, and/or soft tissue plasmacytomas.

3. A method of selectively treating a subject, comprising:
(1) determining whether the subject has a high-risk feature, the high-risk feature being a cytogenetic abnormality, International Staging System (ISS) stage III, and/or soft tissue plasmacytomas; and
(2) administering to the subject who is determined to have the high-risk feature in step (1) a dose of T cells comprising a chimeric antigen receptor (CAR) comprising:
   (a) an extracellular antigen binding domain capable of specifically binding to an epitope of BCMA,
   (b) a transmembrane domain, and
   (c) an intracellular signaling domain,
wherein optionally the subject has multiple myeloma, has received one to three prior lines of therapies, including a therapy with an IMiD, and is refractory to the IMiD.

4. A method of selectively treating a subject, comprising administering to the subject who has been determined to have a high-risk feature a dose of T cells comprising a chimeric antigen receptor (CAR) comprising:
(a) an extracellular antigen binding domain capable of specifically binding to an epitope of BCMA,
(b) a transmembrane domain, and
(c) an intracellular signaling domain,
wherein the high-risk feature is a cytogenetic abnormality, International Staging System (ISS) stage III, and/or soft tissue plasmacytomas,
wherein optionally the subject has multiple myeloma, has received one to three prior lines of therapies, including a therapy with an IMiD, and is refractory to the IMiD.

5. The method of any one of embodiments 1-4, wherein the IMiD is lenalidomide.

6. The method of any one of embodiments 2-5, wherein the high-risk feature is a cytogenetic abnormality.

7. The method of embodiment 6, wherein the cytogenetic abnormality is a high-risk cytogenetic abnormality.

8. The method of embodiment 7, wherein the subject has one or more high-risk cytogenetic abnormality selected from a group comprising Gain/amp(1q), del(17p), t(4;14), t(14;16), or any combination thereof.

9. The method of embodiment 8, wherein the cytogenetic abnormality comprises Gain/amp(1q).

10. The method of embodiment 8, wherein the cytogenetic abnormality comprises del(17p).

11. The method of embodiment 8, wherein the cytogenetic abnormality comprises t(4;14).

12. The method of embodiment 8, wherein the cytogenetic abnormality comprises t(14;16).

13. The method of any one of embodiments 6-12, wherein the subject has at least two cytogenetic abnormalities, and wherein optionally the subject has two, three, four, five, or more cytogenetic abnormalities.

14. The method of embodiment 6, wherein the cytogenetic abnormality is a standard-risk cytogenetic abnormality.

15. The method of any one of embodiments 2-5, wherein the high-risk feature is International Staging System (ISS) stage III.

16. The method of any one of embodiments 2-5, wherein the high-risk feature is soft tissue plasmacytomas.

17. The method of any one of embodiments 1-16, wherein the subject has received one prior line of therapy.

18. The method of any one of embodiments 1-16, wherein the subject has received two prior lines of therapies.

19. The method of any one of embodiments 1-16, wherein the subject has received three prior lines of therapies.

20. The method of any one of embodiments 1-19, wherein the one, two or three prior lines of therapy comprises treatment with pomalidomide.

21. The method of any one of embodiments 1-20, wherein the one, two or three prior lines of therapy further comprises treatment with an anti-CD38 antibody, and wherein optionally the anti-CD38 antibody is daratumumab and/or isatuximab.

22. The method of any one of embodiments 1-21, wherein the one, two or three prior lines of therapy further comprises treatment with a proteasome inhibitor, wherein optionally the proteasome inhibitor is bortezomib, carfilzomib, ixazomib, or any combination thereof.

23. The method of any one of embodiments 1-22, wherein the subject has further received a bridging therapy, wherein optionally the bridging therapy is of the physician's choice, wherein optionally the bridging therapy comprises pomalidomide, bortezomib, dexamethasone, daratumumab, or any combination thereof, further wherein optionally the bridging therapy comprises pomalidomide, bortezomib and dexamethasone, and further wherein optionally the bridging therapy comprises daratumumab, pomalidomide and dexamethasone.

24. The method of embodiment 23, wherein the subject has received the bridging therapy from about every 20 days to about every 30 days, wherein optionally the subject has received the bridging therapy about every 21 days, wherein optionally the subject has received the bridging therapy about every 28 days, and further wherein optionally the subject has received at least one, two, three, four, or more bridging therapies.

25. The method of any one of embodiments 1-24, wherein the subject has further received a lymphodepletion therapy, wherein optionally the lymphodepletion therapy comprises cyclophosphamide and/or fludarabine daily, wherein optionally the lymphodepletion therapy comprises cyclophosphamide and fludarabine daily, further wherein optionally the lymphodepletion therapy comprises cyclophosphamide at a concentration of about 300 mg/m² and fludarabine at a concentration of about 30 mg/m² daily for 3 days.

26. The method of any one of embodiments 1-25, wherein the dose of the T cells is 0.5-1.0×10⁶ cells/kg of body weight of the subject, wherein optionally the dose of the T cells is about 0.75×10⁶ cells/kg of body weight of the subject, wherein optionally the method comprises administering the dose of the T cells about 5 to about 7 days after the start of the lymphodepletion therapy, wherein optionally, the dose is administered as a single infusion.

27. The method of any one of embodiments 1-26, wherein the method is effective in obtaining an overall response in the subject after administering to the subject the dose of the T cells, wherein optionally the method is effective in obtaining the overall response at a rate of about 75% to about 100%, further wherein optionally the method is effective in obtaining the overall response at a rate of about 84.6%, and further wherein optionally the method is effective in obtaining the overall response at a rate of about 99.4%.

28. The method of embodiment 27, wherein the overall response comprises, in order from best to worst:
(1) a stringent complete response;
(2) a complete response;
(3) a very good partial response;
(4) a partial response; or
(5) a minimal response.

29. The method of embodiment 28, wherein the overall response is a stringent complete response, wherein optionally the method is effective in obtaining the stringent complete response at a rate of about 40% to about 90%, about 50% to about 80%, about 58.2%, or about 68.8%.

30. The method of embodiment 28, wherein the overall response is a complete response, wherein optionally the method is effective in obtaining the complete response at a rate of about 10% to about 20%, further wherein optionally the method is effective in obtaining the complete response at a rate of about 14.9% or about 17.6%.

31. The method of embodiment 28, wherein the overall response is a very good partial response or a partial response.

32. The method of embodiment 28, wherein:
(1) the method is effective in obtaining a stringent complete response or a complete response at a rate of about 70% to about 90%, wherein optionally the method is effective in obtaining a stringent complete response or a complete response at a rate of about 73.1% or about 86.4%;
(2) the method is effective in obtaining a stringent complete response, a complete response, or a very good partial response at a rate of about 80% to about 100%, wherein optionally the method is effective in obtaining a stringent complete response, a complete response, or a very good partial response at a rate of about 81.3% or about 96.0%;
(3) the method is effective in obtaining a minimal response;
(4) the method is effective in further obtaining minimal residual disease negative, wherein optionally the method is effective in obtaining minimal residual disease negative at a rate of about 50% to about 80%, further wherein optionally the method is effective in obtaining minimal residual disease negative at a rate of about 60.6% or about 71.6%; or
(5) the method is effective in further obtaining 12-month progression-free survival for at least about 60 to about 100% of subjects, at least about 69.4 to about 81.1% of subjects, or at least about 84.1 to about 93.4% of subjects, wherein optionally the method is effective in obtaining 12-month progression-free survival for at least about 75.9% of subjects or about 89.7% of subjects.

33. The method of any one of embodiments 27-32, wherein:
(1) time to first overall response or first minimal response ranges about 0.9 to about 11.1 months, wherein optionally the time to the first overall response or the first minimal response is at a median of about 2.1 months; or
(2) time to best overall response or best minimal response is about 1.1 to about 18.6 months, wherein optionally the time to the best overall response or the best minimal response is at a median of about 6.4 or about 6.5 months.

34. The method of any one of embodiments 1-33, wherein the method further comprises treating the subject for an adverse event after administering the dose of the T cells, wherein optionally the method comprises administering a treatment to the subject to alleviate the adverse event, wherein optionally the adverse event comprises a hematologic adverse event, a nonhematologic adverse event, a treatment-emergent adverse event, or any combination thereof, wherein optionally the nonhematologic adverse event comprises an infection and/or a nonhematologic adverse event other than an infection, further wherein optionally the adverse event comprises neutropenia, thrombocytopenia, anemia, lymphopenia, an upper respiratory tract infection, nasopharyngitis, sinusitis, rhinitis, tonsillitis, pharyngitis, laryngitis, pharyngotonsillitis, COVID-19, COVID-19 pneumonia, asymptomatic COVID-19, neutropenic sepsis, progressive multifocal leukoencephalpathy, septic shock, respiratory failure, pulmonary embolism, a lower respiratory tract/lung infection, pneumonia, bronchitis, nausea, hypogammaglobulinemia, diarrhea, fatigue, headache, constipation, hypokalemia, asthenia, peripheral edema, decreased appetite, peripheral sensory neuropathy, back pain, arthralgia, pyrexia, dyspnea, insomnia, or any combination thereof, further wherein optionally the adverse event is a Grade 3/4 adverse event, and further wherein optionally the adverse event lasts longer than about 30 days or about 60 days.

35. The method of any one of embodiments 1-34, wherein the method further comprises treating the subject for a second primary malignancy after administering the dose of the T cells, wherein optionally the method comprises administering a treatment to the subject to alleviate the second primary malignancy, wherein optionally the second primary malignancy comprises a cutaneous/non-invasive malignancy, a hematologic malignancy, a non-cutaneous/invasive malignancy, or any combination thereof, further wherein optionally the second primary malignancy comprises basal cell carcinoma, Bowen's disease, lip squamous cell carcinoma, malignant melanoma, malignant melanoma in situ, squamous cell carcinoma of skin, acute myeloid leukemia, a myelodysplastic syndrome, peripheral T-cell lymphoma, angiosarcoma, invasive lobular breast carcinoma, pleomorphic malignant fibrous histiocytoma, renal cell carcinoma, tonsil cancer, or any combination thereof.

36. The method of embodiment 34 or 35, wherein the adverse event or the second primary malignancy occurs in the subject at a rate comparable to a rate of a same adverse event or a same second primary malignancy occurring in a subject undergoing a standard of care.

37. The method of any one of embodiments 1-36, wherein the method further comprises treating the subject for a CAR-T-associated adverse event after administering the dose of the T cells, wherein optionally the method comprises administering a treatment to the subject to alleviate the CAR-T-associated adverse event, wherein optionally the CAR-T-associated adverse event comprises a cytokine release syndrome (CRS) and/or neurotoxicity, wherein optionally:
(a) the CAR-T-associated adverse event is a CRS, further wherein optionally the CRS occurs in the subject at a rate of about 60% to about 90%, or a rate of about 76.1%, further wherein optionally maximum toxicity grade of the CRS is Grade 1, Grade 2 or Grade 3, further wherein optionally:
   (1) the maximum toxicity grade of the CRS is Grade 1, wherein optionally the maximum toxicity grade of Grade 1 in the subject occurs at a rate of about 52.8%;
   (2) the maximum toxicity grade of the CRS is Grade 2, wherein optionally the maximum toxicity grade of Grade 2 in the subject occurs at a rate of about 22.2%;
   (3) the maximum toxicity grade of the CRS is Grade 3, wherein optionally the maximum toxicity grade of Grade 3 in the subject occurs at a rate of about 1.1%;
   (4) time to first onset of the CRS ranges from about 1 to about 23 days, wherein optionally the time to the first onset of the CRS is at a median of about 8 days;
   (5) duration of the CRS ranges from about 1 to about 17 days, wherein optionally the duration of the CRS is at a median of about 3 days; or
   (6) the treatment comprises tocilizumab, oxygen, a corticosteroid, a vasopressor, or any combination thereof; or
(b) the CAR-T-associated adverse event is neurotoxicity, wherein optionally the neurotoxicity comprises an immune effector cell-associated neurotoxicity syndrome or associated symptom, movement and neurocognitive neurotoxicity, treatment-emergent adverse event of neurotoxicity, a non-immune effector cell-associated neurotoxicity syndrome or associated symptom, or any combination thereof, wherein optionally the neurotoxicity is an immune effector cell-associated neurotoxicity syndrome or associated symptom, further wherein optionally:
   (1) the immune effector cell-associated neurotoxicity syndrome or associated symptom in the subject occurs at a rate of about 4.5%;
   (2) maximum toxicity grade of the immune effector cell-associated neurotoxicity syndrome or associated symptom is Grade 1 or Grade 2, wherein optionally maximum toxicity grade of the immune effector cell-associated neurotoxicity syndrome or associated symptom is Grade 1, further wherein optionally the maximum toxicity grade of Grade 1 in the subject occurs at a rate of about 3.4%, or wherein optionally maximum toxicity grade of the immune effector cell-associated neurotoxicity syndrome or associated symptom is Grade 2, further wherein optionally the maximum toxicity grade of Grade 2 in the subject occurs at a rate of about 1.1%;
   (3) time to onset of the immune effector cell-associated neurotoxicity syndrome or associated symptom ranges from about 6 to about 15 days, wherein optionally the time to the onset of the immune effector cell-associated neurotoxicity syndrome or associated symptom is at a median of about 9.5 days;
   (4) duration of the immune effector cell-associated neurotoxicity syndrome or associated symptom ranges from about 1 to about 6 days, wherein optionally the duration of the immune effector cell-associated neurotoxicity syndrome or associated symptom is at a median of about 2 days; or
   (5) the treatment comprises a corticosteroid and/or tocilizumab.

38. The method of embodiment 37, wherein the neurotoxicity is a CAR-T cell neurotoxicity, wherein optionally the CAR-T cell neurotoxicity in the subject occurs at a rate of about 17.0%, wherein optionally the CAR-T cell neurotoxicity comprises Grade 3/4 neurotoxicity, Grade 5 neurotoxicity, cranial nerve palsy, peripheral neuropathy, a movement and neurocognitive treatment-emergent adverse event, or any combination thereof, further wherein optionally:
(a) the CAR-T cell neurotoxicity is Grade 3/4 neurotoxicity that occurs in the subject at a rate of about 2.3%;
(b) the CAR-T cell neurotoxicity is Grade 5 neurotoxicity;
(c) the CAR-T cell neurotoxicity is cranial nerve palsy, wherein optionally the cranial nerve palsy in the subject occurs at a rate of about 9.1%, wherein optionally:
   (1) the cranial nerve palsy is Grade 2 or Grade 3 cranial nerve palsy, further wherein optionally the cranial nerve palsy is Grade 2 cranial nerve palsy that occurs in the subject at a rate of about 8.0%, and further wherein optionally the cranial nerve palsy is Grade 3 cranial nerve palsy that occurs in the subject at a rate of about 1.1%;
   (2) time to onset of the cranial nerve palsy after administering the dose of the T cells to the subject ranges from about 17 days to about 60 days, further wherein optionally the time to onset of the cranial nerve palsy after administering the dose of the T cells to the subject is at a median of about 21 days;
   (3) the cranial nerve palsy affects cranial nerve III, V, or VII;
   (4) duration of the cranial nerve palsy ranges from about 15 days to about 262 days, further wherein optionally duration of the cranial nerve palsy is at a median of about 77 days; or
   (5) the treatment comprises a corticosteroid;
(d) the CAR-T cell neurotoxicity is peripheral neuropathy, wherein optionally the peripheral neuropathy in the subject occurs at a rate of about 2.8%; or
(e) the CAR-T cell neurotoxicity is a movement and neurocognitive treatment-emergent adverse event, wherein optionally the movement and neurocognitive treatment-emergent adverse event is Grade 1, further wherein optionally the Grade 1 movement and neurocognitive treatment-emergent adverse event in the subject occurs at a rate of about 0.6%.

39. The method of any one of embodiments 1-38, wherein:
(a) CD3+ cells comprising the CAR in the blood of the subject peak at a median of about 13 days after administering the T cells to the subject, wherein optionally the CD3+ cells comprising the CAR in the blood of the subject peak at a mean concentration of about 1523 cells/μL;
(b) CD3+ cells comprising the CAR in the blood of the subject remain detectable from about 13 days to about 631 days after administering the T cells to the subject, wherein optionally the CD3+ cells comprising the CAR in the blood of the subject remain detectable at a median of about 57 days after administering the T cells to the subject; or
(c) AUC₀₋₂₈ of CD3+ cells comprising the CAR in the blood of the subject is at a mean value of about 12,504 cells/μL.

40. The method of any one of embodiments 1-39, wherein the first VHH domain comprising a CDR1, a CDR2, and a CDR3 as set forth in the VHH domain comprising the amino acid sequence of SEQ ID NO: 2, and the second VHH domain comprising a CDR1, a CDR2, and a CDR3 as set forth in the VHH domain comprising the amino acid sequence of SEQ ID NO: 4, wherein optionally the first VHH domain comprises a CDR1 comprising the amino acid sequence of SEQ ID NO: 18, a CDR2 comprising the amino acid sequence of SEQ ID NO: 19, a CDR3 comprising the amino acid sequence of SEQ ID NO: 20, and the second VHH domain comprises a CDR1 comprising the amino acid sequence of SEQ ID NO: 21, a CDR2 comprising the amino acid sequence of SEQ ID NO: 22, and a CDR3 comprising the amino acid sequence of SEQ ID NO: 23, wherein optionally the first VHH domain comprises the amino acid sequence of SEQ ID NO: 2 and the second VHH domain comprises the amino acid sequence of SEQ ID NO: 4, wherein optionally the first VHH domain is at the N-terminus of the second VHH domain, or the first VHH domain is at the C-terminus of the second VHH domain, further wherein optionally:
(a) the first VHH domain is linked to the second VHH domain via a linker comprising the amino acid sequence of SEQ ID NO: 3;
(b) the transmembrane domain is derived from a molecule selected from the group consisting of CD8α, CD4, CD28, CD137, CD80, CD86, CD152 and PD1, wherein optionally the transmembrane domain is derived from CD8a and comprises the amino acid sequence of SEQ ID NO: 6;
(c) the intracellular signaling domain comprises a primary intracellular signaling domain of an immune effector cell, wherein optionally the primary intracellular signaling domain is derived from CD3ζ comprising the amino acid sequence of SEQ ID NO: 8;
(d) the intracellular signaling domain comprises a co-stimulatory signaling domain, wherein optionally the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 and any combination thereof, wherein optionally the co-stimulatory signaling domain comprises a cytoplasmic domain of CD137 comprising the amino acid sequence of SEQ ID NO: 7;
(e) the CAR further comprises a hinge domain located between the C-terminus of the extracellular antigen binding domain and the N-terminus of the transmembrane domain, wherein optionally the hinge domain is derived from CD8a comprising the amino acid sequence of SEQ ID NO: 5;
(f) the CAR further comprises a signal peptide located at the N-terminus of the polypeptide, wherein optionally the signal peptide is derived from CD8a comprising the amino acid sequence of SEQ ID NO: 1; or
(g) the CAR comprises the amino acid sequence of SEQ ID NO: 17.

41. The method of any one of embodiments 1-40, wherein the dose of T cells is formulated in a composition comprising 5% dimethyl sulfoxide (DMSO).

42. The method of any one of embodiments 1-41, wherein the administration of the dose of T cells reduces the risk of disease progression or death in the subject.

43. The method of embodiment 42, wherein the risk of disease progression or death is reduced relative to an administration of daratumumab-pomalidomide-dexamethasone (DPd) or pomalidomide-bortezomib-dexamethasone (PVd) treatment.

44. The method of embodiment 42, wherein the risk of disease progression or death is reduced relative to an administration of ide-cel treatment.

45. The method of any one of embodiments 42-44, wherein the subject has an about 60% to about 75% reduced risk of disease progression or death.

46. The method of embodiments 45, wherein the subject has an about 74% reduced risk of disease progression or death.

47. The method of any one of embodiments 42-46, wherein the IMiD is lenalidomide.

48. The method of any one of embodiments 42-47 wherein the subject has received three or fewer prior lines of therapy.

49. The method of any one of embodiments 42-47, wherein the subject has received two or fewer prior lines of therapy.

50. The method of any one of embodiments 42-47, wherein the subject has received only one prior line of therapy.

51. The method of any one of embodiments 42-50, wherein the method is effective in obtaining an overall response rate (ORR) about 75% to about 100%.

52. The method of embodiment 51, wherein the ORR is about 84.6%.

53. The method of any one of embodiments 42-52, wherein the treatment is effective in prolonging the median progression-free survival (PFS) time of the subject as compared to an administration of DPd or PVd treatment.

54. The method of embodiment 53, wherein the PFS at 12 months after administration of the treatment is about 75.9%.

55. The method of embodiment 54, wherein the PFS at 12 months after administration of DPd or PVd is about 48.6%.

56. The method of any one of embodiments 1-23 and 42-55, wherein the treatment is more effective in obtaining a stringent complete response (sCR) in the subject as compared to an administration of DPd or PVd treatment.

57. The method of embodiment 56, wherein the sCR after administration of the treatment is about 58.2%.

58. The method of embodiment 57, wherein the sCR after administration of DPd or PVd is about 15.2%.

59. The method of any one of embodiments 1-23 and 42-58, wherein the treatment is more effective in obtaining a very good partial response (VGPR) or better in the subject as compared to an administration of DPd or PVd treatment.

60. The method of embodiment 59, wherein the VGPR or better response after administration of the treatment is about 81.3%.

61. The method of embodiment 60, wherein the VGPR or better response after administration of DPd or PVd is about 45.5%.

62. The method of any one of embodiments 42-61, wherein the method further comprises treating the subject for a CAR-T-associated adverse event after administering the dose of the T cells, wherein optionally the method comprises administering a treatment to the subject to alleviate the CAR-T-associated adverse event, wherein optionally the CAR-T-associated adverse event comprises a cytokine release syndrome (CRS) and/or neurotoxicity, wherein optionally:
(a) the CAR-T-associated adverse event is a CRS, further wherein optionally the CRS occurs in the subject at a rate of about 60% to about 90%, or a rate of about 76.1%, further wherein optionally maximum toxicity grade of the CRS is Grade 1, Grade 2 or Grade 3, further wherein optionally:
   (1) the maximum toxicity grade of the CRS is Grade 1, wherein optionally the maximum toxicity grade of Grade 1 in the subject occurs at a rate of about 52.8%;
   (2) the maximum toxicity grade of the CRS is Grade 2, wherein optionally the maximum toxicity grade of Grade 2 in the subject occurs at a rate of about 22.2%;
   (3) the maximum toxicity grade of the CRS is Grade 3, wherein optionally the maximum toxicity grade of Grade 3 in the subject occurs at a rate of about 1.1%;
   (4) time to first onset of the CRS ranges from about 1 to about 23 days, wherein optionally the time to the first onset of the CRS is at a median of about 8 days;
   (5) duration of the CRS ranges from about 1 to about 17 days, wherein optionally the duration of the CRS is at a median of about 3 days; or
   (6) the treatment comprises tocilizumab, oxygen, a corticosteroid, a vasopressor, or any combination thereof; or
(b) the CAR-T-associated adverse event is neurotoxicity, wherein optionally the neurotoxicity comprises an immune effector cell-associated neurotoxicity syndrome or associated symptom, movement and neurocognitive neurotoxicity, treatment-emergent adverse event of neurotoxicity, a non-immune effector cell-associated neurotoxicity syndrome or associated symptom, or any combination thereof, wherein optionally the neurotoxicity is an immune effector cell-associated neurotoxicity syndrome or associated symptom, further wherein optionally:
   (1) the immune effector cell-associated neurotoxicity syndrome or associated symptom in the subject occurs at a rate of about 4.5%;
   (2) maximum toxicity grade of the immune effector cell-associated neurotoxicity syndrome or associated symptom is Grade 1 or Grade 2, wherein optionally maximum toxicity grade of the immune effector cell-associated neurotoxicity syndrome or associated symptom is Grade 1, further wherein optionally the maximum toxicity grade of Grade 1 in the subject occurs at a rate of about 3.4%, or wherein optionally maximum toxicity grade of the immune effector cell-associated neurotoxicity syndrome or associated symptom is Grade 2, further wherein optionally the maximum toxicity grade of Grade 2 in the subject occurs at a rate of about 1.1%;
   (3) time to onset of the immune effector cell-associated neurotoxicity syndrome or associated symptom ranges from about 6 to about 15 days, wherein optionally the time to the onset of the immune effector cell-associated neurotoxicity syndrome or associated symptom is at a median of about 9.5 days;
   (4) duration of the immune effector cell-associated neurotoxicity syndrome or associated symptom ranges from about 1 to about 6 days, wherein optionally the duration of the immune effector cell-associated neurotoxicity syndrome or associated symptom is at a median of about 2 days; or
   (5) the treatment comprises a corticosteroid and/or tocilizumab.

63. The method of embodiment 62, wherein the neurotoxicity is a CAR-T cell neurotoxicity, wherein optionally the CAR-T cell neurotoxicity in the subject occurs at a rate of about 17.0%, wherein optionally the CAR-T cell neurotoxicity comprises Grade 3/4 neurotoxicity, Grade 5 neurotoxicity, cranial nerve palsy, peripheral neuropathy, a movement and neurocognitive treatment-emergent adverse event, or any combination thereof, further wherein optionally:
(a) the CAR-T cell neurotoxicity is Grade 3/4 neurotoxicity that occurs in the subject at a rate of about 2.3%;
(b) the CAR-T cell neurotoxicity is Grade 5 neurotoxicity;
(c) the CAR-T cell neurotoxicity is cranial nerve palsy, wherein optionally the cranial nerve palsy in the subject occurs at a rate of about 9.1%, wherein optionally:
   (1) the cranial nerve palsy is Grade 2 or Grade 3 cranial nerve palsy, further wherein optionally the cranial nerve palsy is Grade 2 cranial nerve palsy that occurs in the subject at a rate of about 8.0%, and further wherein optionally the cranial nerve palsy is Grade 3 cranial nerve palsy that occurs in the subject at a rate of about 1.1%;
   (2) time to onset of the cranial nerve palsy after administering the dose of the T cells to the subject ranges from about 17 days to about 60 days, further wherein optionally the time to onset of the cranial nerve palsy after administering the dose of the T cells to the subject is at a median of about 21 days;
   (3) the cranial nerve palsy affects cranial nerve III, V, or VII;
   (4) duration of the cranial nerve palsy ranges from about 15 days to about 262 days, further wherein optionally duration of the cranial nerve palsy is at a median of about 77 days; or
   (5) the treatment comprises a corticosteroid;
(d) the CAR-T cell neurotoxicity is peripheral neuropathy, wherein optionally the peripheral neuropathy in the subject occurs at a rate of about 2.8%; or
(e) the CAR-T cell neurotoxicity is a movement and neurocognitive treatment-emergent adverse event, wherein optionally the movement and neurocognitive treatment-emergent adverse event is Grade 1, further wherein optionally the Grade 1 movement and neurocognitive treatment-emergent adverse event in the subject occurs at a rate of about 0.6%.

64. The method of any one of embodiments 42-63, wherein the subject has one or more high-risk cytogenetic abnormality selected from a group comprising Gain/amp(1q), del(17p), t(4;14), t(14;16), or any combination thereof.

65. The method of embodiment 64, wherein the subject has at least two cytogenetic abnormalities, and wherein optionally the subject has two, three, four, five, or more cytogenetic abnormalities.

66. The method of embodiment 64 or 65, wherein the cytogenetic abnormality is a standard-risk cytogenetic abnormality.

67. The method of any embodiments of 1-66, wherein the administration of the dose of T cells is more effective in obtaining a greater very good partial response (VGPR) or better in the subject as compared to an administration of DPd or PVd treatment.

68. The method of embodiment 67, wherein the VGPR or better response after administration of the treatment is about 81.3%.

69. The method of embodiment 68, wherein the VGPR or better response after administration of DPd or PVd is about 45.5%.

70. The method of any one of embodiments 67-69, wherein the treatment is more effective in obtaining a stringent complete response (sCR) in the subject as compared to an administration of DPd or PVd treatment.

71. The method of embodiment 70 wherein the sCR after administration of the treatment is about 58.2%.

72. The method of embodiment 71, wherein the sCR after administration of DPd or PVd is about 15.2%.

73. The method of any one of embodiments 67-72, wherein the administration of the dose of T cells reduces the risk of disease progression or death in the subject.

74. The method of embodiment 73, wherein the risk of disease progression or death is reduced relative to an administration of daratumumab-pomalidomide-dexamethasone (DPd) or pomalidomide-bortezomib-dexamethasone (PVd) treatment.

75. The method of embodiment 73, wherein the risk of disease progression or death is reduced relative to an administration of ide-cel treatment.

76. The method of any one of embodiments 67-75, wherein the subject has an about 60% to about 75% reduced risk of disease progression or death.

77. The method of embodiment 76, wherein the subject has an about 74% reduced risk of disease progression or death.

78. The method of any one of embodiments 67-77, wherein the IMiD is lenalidomide.

79. The method of any one of embodiments 67-78 wherein the subject has received three or fewer prior lines of therapy.

80. The method of any one of embodiments 67-79, wherein the subject has received two or fewer prior lines of therapy.

81. The method of any one of embodiments 67-80, wherein the subject has received only one prior line of therapy.

82. The method of any one of embodiments 67-81, wherein the method is effective in obtaining an overall response rate (ORR) of about 75% to about 100%.

83. The method of embodiment 82, wherein the ORR is about 84.6%.

84. The method of any one of embodiments 67-83, wherein the treatment is effective in prolonging the median progression-free survival (PFS) time of the subject as compared to an administration of DPd or PVd treatment.

85. The method of embodiment 84, wherein the PFS at 12 months after administration of the treatment is about 75.9%.

86. The method of embodiment 85, wherein the PFS at 12 months after administration of DPd or PVd is about 48.6%.

87. The method of any one of embodiments 67-86, wherein the method further comprises treating the subject for a CAR-T-associated adverse event after administering the dose of the T cells, wherein optionally the method comprises administering a treatment to the subject to alleviate the CAR-T-associated adverse event, wherein optionally the CAR-T-associated adverse event comprises a cytokine release syndrome (CRS) and/or neurotoxicity, wherein optionally:
(a) the CAR-T-associated adverse event is a CRS, further wherein optionally the CRS occurs in the subject at a rate of about 60% to about 90%, or a rate of about 76.1%, further wherein optionally maximum toxicity grade of the CRS is Grade 1, Grade 2 or Grade 3, further wherein optionally:
   (1) the maximum toxicity grade of the CRS is Grade 1, wherein optionally the maximum toxicity grade of Grade 1 in the subject occurs at a rate of about 52.8%;
   (2) the maximum toxicity grade of the CRS is Grade 2, wherein optionally the maximum toxicity grade of Grade 2 in the subject occurs at a rate of about 22.2%;
   (3) the maximum toxicity grade of the CRS is Grade 3, wherein optionally the maximum toxicity grade of Grade 3 in the subject occurs at a rate of about 1.1%;
   (4) time to first onset of the CRS ranges from about 1 to about 23 days, wherein optionally the time to the first onset of the CRS is at a median of about 8 days;
   (5) duration of the CRS ranges from about 1 to about 17 days, wherein optionally the duration of the CRS is at a median of about 3 days; or
   (6) the treatment comprises tocilizumab, oxygen, a corticosteroid, a vasopressor, or any combination thereof; or
(b) the CAR-T-associated adverse event is neurotoxicity, wherein optionally the neurotoxicity comprises an immune effector cell-associated neurotoxicity syndrome or associated symptom, movement and neurocognitive neurotoxicity, treatment-emergent adverse event of neurotoxicity, a non-immune effector cell-associated neurotoxicity syndrome or associated symptom, or any combination thereof, wherein optionally the neurotoxicity is an immune effector cell-associated neurotoxicity syndrome or associated symptom, further wherein optionally:
   (1) the immune effector cell-associated neurotoxicity syndrome or associated symptom in the subject occurs at a rate of about 4.5%;
   (2) maximum toxicity grade of the immune effector cell-associated neurotoxicity syndrome or associated symptom is Grade 1 or Grade 2, wherein optionally maximum toxicity grade of the immune effector cell-associated neurotoxicity syndrome or associated symptom is Grade 1, further wherein optionally the maximum toxicity grade of Grade 1 in the subject occurs at a rate of about 3.4%, or wherein optionally maximum toxicity grade of the immune effector cell-associated neurotoxicity syndrome or associated symptom is Grade 2, further wherein optionally the maximum toxicity grade of Grade 2 in the subject occurs at a rate of about 1.1%;
   (3) time to onset of the immune effector cell-associated neurotoxicity syndrome or associated symptom ranges from about 6 to about 15 days, wherein optionally the time to the onset of the immune effector cell-associated neurotoxicity syndrome or associated symptom is at a median of about 9.5 days;
   (4) duration of the immune effector cell-associated neurotoxicity syndrome or associated symptom ranges from about 1 to about 6 days, wherein optionally the duration of the immune effector cell-associated neurotoxicity syndrome or associated symptom is at a median of about 2 days; or
   (5) the treatment comprises a corticosteroid and/or tocilizumab.

88. The method of embodiment 87, wherein the neurotoxicity is a CAR-T cell neurotoxicity, wherein optionally the CAR-T cell neurotoxicity in the subject occurs at a rate of about 17.0%, wherein optionally the CAR-T cell neurotoxicity comprises Grade 3/4 neurotoxicity, Grade 5 neurotoxicity, cranial nerve palsy, peripheral neuropathy, a movement and neurocognitive treatment-emergent adverse event, or any combination thereof, further wherein optionally:
(a) the CAR-T cell neurotoxicity is Grade 3/4 neurotoxicity that occurs in the subject at a rate of about 2.3%;
(b) the CAR-T cell neurotoxicity is Grade 5 neurotoxicity;
(c) the CAR-T cell neurotoxicity is cranial nerve palsy, wherein optionally the cranial nerve palsy in the subject occurs at a rate of about 9.1%, wherein optionally:
   (1) the cranial nerve palsy is Grade 2 or Grade 3 cranial nerve palsy, further wherein optionally the cranial nerve palsy is Grade 2 cranial nerve palsy that occurs in the subject at a rate of about 8.0%, and further wherein optionally the cranial nerve palsy is Grade 3 cranial nerve palsy that occurs in the subject at a rate of about 1.1%;
   (2) time to onset of the cranial nerve palsy after administering the dose of the T cells to the subject ranges from about 17 days to about 60 days, further wherein optionally the time to onset of the cranial nerve palsy after administering the dose of the T cells to the subject is at a median of about 21 days;
   (3) the cranial nerve palsy affects cranial nerve III, V, or VII;
   (4) duration of the cranial nerve palsy ranges from about 15 days to about 262 days, further wherein optionally duration of the cranial nerve palsy is at a median of about 77 days; or
   (5) the treatment comprises a corticosteroid;
(d) the CAR-T cell neurotoxicity is peripheral neuropathy, wherein optionally the peripheral neuropathy in the subject occurs at a rate of about 2.8%; or
(e) the CAR-T cell neurotoxicity is a movement and neurocognitive treatment-emergent adverse event, wherein optionally the movement and neurocognitive treatment-emergent adverse event is Grade 1, further wherein optionally the Grade 1 movement and neurocognitive treatment-emergent adverse event in the subject occurs at a rate of about 0.6%.

89. The method of any one of embodiments 67-88, wherein the subject has one or more high-risk cytogenetic abnormality selected from a group comprising Gain/amp(1q), del(17p), t(4;14), t(14;16), or any combination thereof.

90. The method of embodiment 89, wherein the subject has at least two cytogenetic abnormalities, and wherein optionally the subject has two, three, four, five, or more cytogenetic abnormalities.

91. The method of embodiments 89 or 90, wherein the cytogenetic abnormality is a standard-risk cytogenetic abnormality.

92. The method of any one of embodiments 1-91, wherein the method further comprises treating the subject for a CAR-T-associated adverse event after administering the dose of the T cells, wherein the CAR-T-associated adverse event comprises a cytokine release syndrome (CRS) and/or neurotoxicity.

93. The method of embodiment 92, wherein the CRS CAR-T-associated adverse event is a CRS with a maximum toxicity grade of Grade 1, wherein optionally the maximum toxicity grade of Grade 1 in the subject occurs at a rate of about 52.8%.

94. The method of embodiment 92, wherein the CRS CAR-T-associated adverse event is a CRS with a maximum toxicity grade of Grade 2, wherein optionally the maximum toxicity grade of Grade 2 in the subject occurs at a rate of about 22.2%.

95. The method of embodiment 92, wherein the CRS CAR-T-associated adverse event is a CRS with a maximum toxicity grade of Grade 3, wherein optionally the maximum toxicity grade of Grade 3 in the subject occurs at a rate of about 1.1%.

96. The method of any one of embodiments 93-95, wherein the time to first onset of the CRS ranges from about 1 to about 23 days, wherein optionally the time to the first onset of the CRS is at a median of about 8 days.

97. The method of any one of embodiments 93-96, wherein the duration of the CRS ranges from about 1 to about 17 days, wherein optionally the duration of the CRS is at a median of about 3 days.

98. The method of any one of embodiments 93-97, wherein the treatment comprises tocilizumab, oxygen, a corticosteroid, a vasopressor, or any combination thereof.

99. The method of embodiment 92, wherein the neurotoxicity CAR-T-associated adverse event is selected from the group consisting of an immune effector cell-associated neurotoxicity syndrome or associated symptom, movement and neurocognitive neurotoxicity, treatment-emergent adverse event of neurotoxicity, a non-immune effector cell-associated neurotoxicity syndrome or associated symptom, or any combination thereof.

100. The method of embodiment 92, wherein the neurotoxicity is an immune effector cell-associated neurotoxicity syndrome or associated symptom.

101. The method of embodiment 100, wherein the immune effector cell-associated neurotoxicity syndrome or associated symptom in the subject occurs at a rate of about 4.5%.

102. The method of embodiment 100 or 101, wherein the maximum toxicity grade of the immune effector cell-associated neurotoxicity syndrome or associated symptom is Grade 1, wherein the maximum toxicity grade of Grade 1 in the subject occurs at a rate of about 3.4%.

103. The method of embodiment 100 or 101, wherein the maximum toxicity grade of the immune effector cell-associated neurotoxicity syndrome or associated symptom is Grade 2, wherein the maximum toxicity grade of Grade 1 in the subject occurs at a rate of about 1.1%.

104. The method of any one of embodiments 100-103, wherein the time to onset of the immune effector cell-associated neurotoxicity syndrome or associated symptom ranges from about 6 to about 15 days.

105. The method of embodiment 104, wherein the time to the onset of the immune effector cell-associated neurotoxicity syndrome or associated symptom is at a median of about 9.5 days.

106. The method of any one of embodiments 100-103, wherein the duration of the immune effector cell-associated neurotoxicity syndrome or associated symptom ranges from about 1 to about 6 days.

107. The method of embodiment 106, wherein the duration of the immune effector cell-associated neurotoxicity syndrome or associated symptom is at a median of about 2 days.

108. The method of any one of embodiments 100-107, wherein the treatment comprises a corticosteroid and/or tocilizumab.

109. The method of any one of embodiments 99-108, wherein the CAR-T cell neurotoxicity in the subject occurs at a rate of about 17.0%.

110. The method of any one of embodiments 99-109, wherein the CAR-T cell neurotoxicity is Grade 3/4 neurotoxicity, and wherein the Grade 3/4 neurotoxicity occurs in the subject at a rate of about 2.3%.

111. The method of any one of embodiments 99-109, wherein the CAR-T cell neurotoxicity is Grade 5 neurotoxicity.

112. The method of any one of embodiments 99-109, wherein the CAR-T cell neurotoxicity is cranial nerve palsy, and wherein the cranial nerve palsy in the subject occurs at a rate of about 9.1%.

113. The method of embodiment 112, wherein the cranial nerve palsy is a Grade 2 cranial nerve palsy, wherein the Grade 2 cranial nerve palsy occurs in the subject at a rate of about 8.0%.

114. The method of embodiment 112, wherein the cranial nerve palsy is a Grade 3 cranial nerve palsy, wherein the Grade 2 cranial nerve palsy occurs in the subject at a rate of about 1.1%.

115. The method of any one of embodiments 112-114, wherein the time to onset of the cranial nerve palsy after administering the dose of the T cells to the subject ranges from about 17 days to about 60 days.

116. The method of any one of embodiments 112-115, wherein the cranial nerve palsy affects cranial nerve III, V, or VII.

117. The method of any one of embodiments 112-116, wherein the duration of the cranial nerve palsy ranges from about 15 days to about 262 days.

118. The method of any one of embodiments 112-117, wherein the treatment comprises a corticosteroid.

119. The method of any one of embodiments 99-109, wherein the CAR-T cell neurotoxicity is peripheral neuropathy, and wherein the peripheral neuropathy in the subject occurs at a rate of about 2.8%.

120. The method of any one of embodiments 99-109, wherein the CAR-T cell neurotoxicity is a movement and neurocognitive treatment-emergent adverse event (MNT).

121. The method of embodiment 120, wherein the MNT is a Grade 1 MNT, wherein the Grade 1 MNT in the subject occurs at a rate of about 0.6%.

122. The method of embodiment 64 or 89, wherein the cytogenetic abnormality comprises Gain/amp(1q).

123. The method of embodiment 64 or 89, wherein the cytogenetic abnormality comprises del(17p).

124. The method of embodiment 64 or 89, wherein the cytogenetic abnormality comprises t(4;14).

125. The method of embodiment 64 or 89, wherein the cytogenetic abnormality comprises t(14;16).

126. The method of any one of embodiments 1-91, wherein the subject has a reduced risk for developing a CAR-T-associated adverse event.

127. The method of embodiment 126, wherein the CAR-T-associated adverse event comprises a cytokine release syndrome (CRS).

128. The method of embodiment 127, wherein the CRS occurs in the subject at a rate of about 60% to about 90%, or a rate of about 76.1%.

129. The method of embodiment 127 or 128, wherein maximum toxicity grade of the CRS is Grade 1, Grade 2 or Grade 3.

130. The method of any one of embodiments 126 to 129, wherein the maximum toxicity grade of the CRS is Grade 1, wherein optionally the maximum toxicity grade of Grade 1 in the subject occurs at a rate of about 52.8%.

131. The method of any one of embodiments 126 to 130, wherein the maximum toxicity grade of the CRS is Grade 2, wherein optionally the maximum toxicity grade of Grade 2 in the subject occurs at a rate of about 22.2%.

132. The method of any one of embodiments 126 to 131, wherein the maximum toxicity grade of the CRS is Grade 3, wherein optionally the maximum toxicity grade of Grade 3 in the subject occurs at a rate of about 1.1%.

133. The method of embodiment 126, wherein the CAR-T-associated adverse event comprises a neurotoxicity.

134. The method of embodiment 133, wherein the neurotoxicity CAR-T-associated adverse event is selected from the group consisting of an immune effector cell-associated neurotoxicity syndrome or associated symptom, movement and neurocognitive neurotoxicity, treatment-emergent adverse event of neurotoxicity, a non-immune effector cell-associated neurotoxicity syndrome or associated symptom, or any combination thereof.

135. The method of embodiment 134, wherein the neurotoxicity is an immune effector cell-associated neurotoxicity syndrome or associated symptom.

136. The method of embodiment 135, wherein the immune effector cell-associated neurotoxicity syndrome or associated symptom in the subject occurs at a rate of about 4.5%.

137. The method of embodiment 135 or 136, wherein the maximum toxicity grade of the immune effector cell-associated neurotoxicity syndrome or associated symptom is Grade 1, wherein the maximum toxicity grade of Grade 1 in the subject occurs at a rate of about 3.4%.

138. The method of embodiment 135 or 136, wherein the maximum toxicity grade of the immune effector cell-associated neurotoxicity syndrome or associated symptom is Grade 2, wherein the maximum toxicity grade of Grade 1 in the subject occurs at a rate of about 1.1%.

139. The method of any one of embodiments 135-138, wherein the time to onset of the immune effector cell-associated neurotoxicity syndrome or associated symptom ranges from about 6 to about 15 days.

140. The method of embodiment 139, wherein the time to the onset of the immune effector cell-associated neurotoxicity syndrome or associated symptom is at a median of about 9.5 days.

141. The method of any one of embodiments 135-138, wherein the duration of the immune effector cell-associated neurotoxicity syndrome or associated symptom ranges from about 1 to about 6 days.

142. The method of embodiment 141, wherein the duration of the immune effector cell-associated neurotoxicity syndrome or associated symptom is at a median of about 2 days.

143. The method of any one of embodiments 135-142, wherein the treatment comprises a corticosteroid and/or tocilizumab.

144. The method of any one of embodiments 134-143, wherein the CAR-T cell neurotoxicity in the subject occurs at a rate of about 17.0%.

145. The method of any one of embodiments 134-144, wherein the CAR-T cell neurotoxicity is Grade 3/4 neurotoxicity, and wherein the Grade 3/4 neurotoxicity occurs in the subject at a rate of about 2.3%.

146. The method of any one of embodiments 134-145, wherein the CAR-T cell neurotoxicity is Grade 5 neurotoxicity.

147. The method of any one of embodiments 134-144, wherein the CAR-T cell neurotoxicity is cranial nerve palsy, and wherein the cranial nerve palsy in the subject occurs at a rate of about 9.1%.

148. The method of embodiment 147, wherein the cranial nerve palsy is a Grade 2 cranial nerve palsy, wherein the Grade 2 cranial nerve palsy occurs in the subject at a rate of about 8.0%.

149. The method of embodiment 147, wherein the cranial nerve palsy is a Grade 3 cranial nerve palsy, wherein the Grade 2 cranial nerve palsy occurs in the subject at a rate of about 1.1%.

150. The method of any one of embodiments 147-149, wherein the time to onset of the cranial nerve palsy after administering the dose of the T cells to the subject ranges from about 17 days to about 60 days.

151. The method of any one of embodiments 147-150, wherein the cranial nerve palsy affects cranial nerve III, V, or VII.

152. The method of any one of embodiments 147-151, wherein the duration of the cranial nerve palsy ranges from about 15 days to about 262 days.

153. The method of any one of embodiments 147-152, wherein the treatment comprises a corticosteroid.

154. The method of any one of embodiments 134-144, wherein the CAR-T cell neurotoxicity is peripheral neuropathy, and wherein the peripheral neuropathy in the subject occurs at a rate of about 2.8%.

155. The method of any one of embodiments 134-144, wherein the CAR-T cell neurotoxicity is a movement and neurocognitive treatment-emergent adverse event (MNT).

156. The method of embodiment 155, wherein the MNT is a Grade 1 MNT, wherein the Grade 1 MNT in the subject occurs at a rate of about 0.6%.

157. The method of any one of embodiments 38, 63, 88, 119, and 154, wherein the peripheral neuropathy is Grade 1, and wherein the Grade 1 peripheral neuropathy occurs at a rate of about 1.1%.

158. The method of any one of embodiments 38, 63, 88, 119, and 154, wherein the peripheral neuropathy is Grade 2, and wherein the Grade 2 peripheral neuropathy occurs at a rate of about 1.1%.

159. The method of any one of embodiments 38, 63, 88, 119, and 154, wherein the peripheral neuropathy is Grade 3, and wherein the Grade 3 peripheral neuropathy occurs at a rate of about 0.6%.

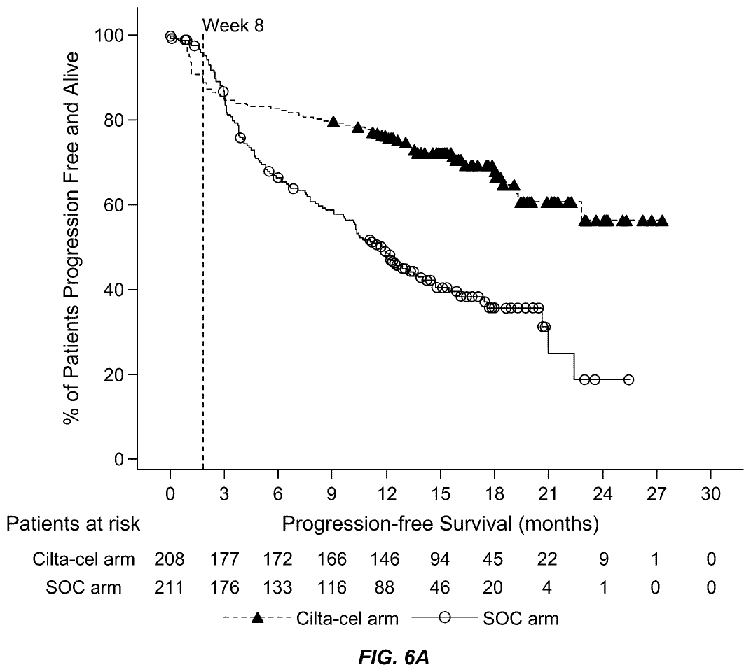

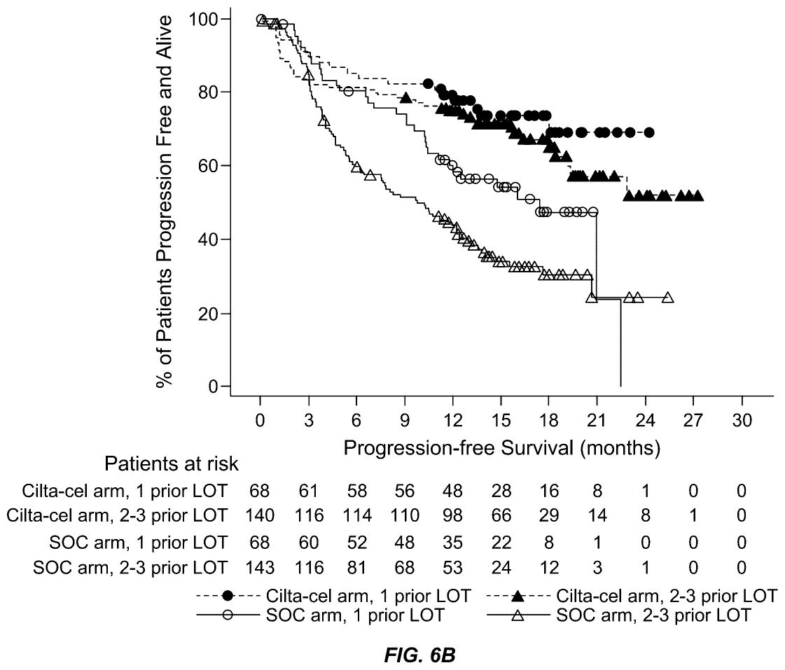

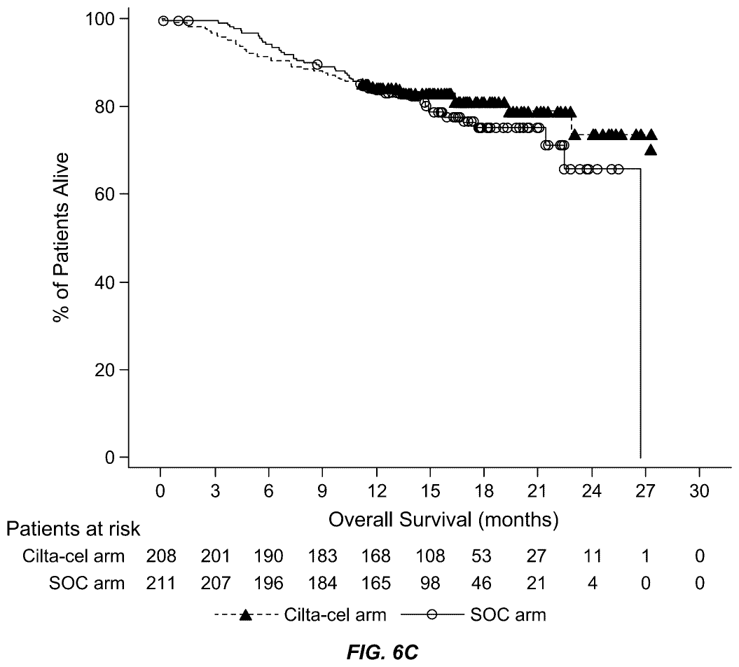

160. A method of selectively treating a subject, comprising administering to the subject who has been determined to have a high-risk feature a dose of T cells comprising a chimeric antigen receptor (CAR) comprising:
(a) an extracellular antigen binding domain capable of specifically binding to an epitope of BCMA,
(b) a transmembrane domain, and
(c) an intracellular signaling domain,
wherein the CAR comprises the amino acid sequence of SEQ ID NO: 17,
wherein the high-risk feature is a cytogenetic abnormality, International Staging System (ISS) stage III, and/or soft tissue plasmacytomas,
wherein the subject has multiple myeloma, has received one to three prior lines of therapies, including a therapy with an IMiD, and is refractory to the IMiD,
wherein the method is effective in obtaining an overall response as described in Example 3, any one of Tables 1-5 and 15, or any one of FIGS. 6A-6C, 7, and 8A-8D,
wherein optionally the method further comprises treating the subject for an adverse event after administering the dose of the T cells as described in Example 4, or any one of Tables 6-14 and 16, and
wherein optionally the method further comprises administering a treatment to the subject to alleviate the adverse event as described in Example 4, or any one of Tables 6-14 and 16.

### EXAMPLES

The following examples are provided to further describe some of the aspects and embodiments disclosed herein. The examples are intended to illustrate, not to limit, the disclosed aspects or embodiments.

### Example 1: Ciltacabtagene Autoleucel

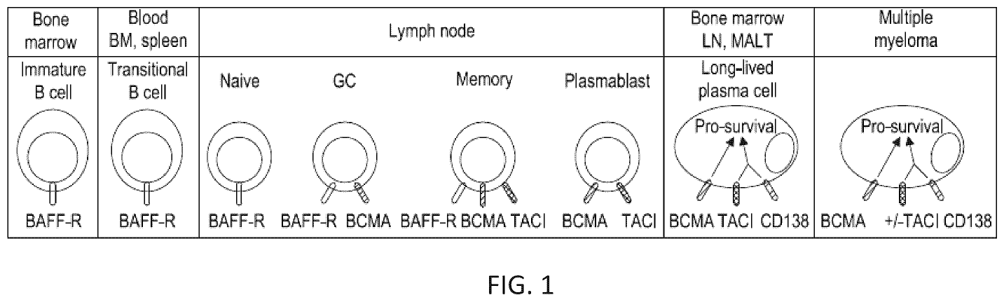

B cell maturation antigen (BCMA, also known as CD269 and TNFRSF17) is a 20 kilodalton, type III membrane protein that is part of the tumor necrosis receptor superfamily. BCMA is a cell surface antigen that is predominantly expressed in B-lineage cells at high levels. FIG. 1 shows the expression of BCMA on various immune-derived cells. Comparative studies have shown a lack of BCMA in most normal tissues and absence of expression on CD34-positive hematopoietic stem cells. BCMA binds 2 ligands that induce B cell proliferation, and plays a critical role in B cell maturation and subsequent differentiation into plasma cells. The selective expression and the biological importance for the proliferation and survival of myeloma cells makes BCMA a promising target for CAR-T based immunotherapy.

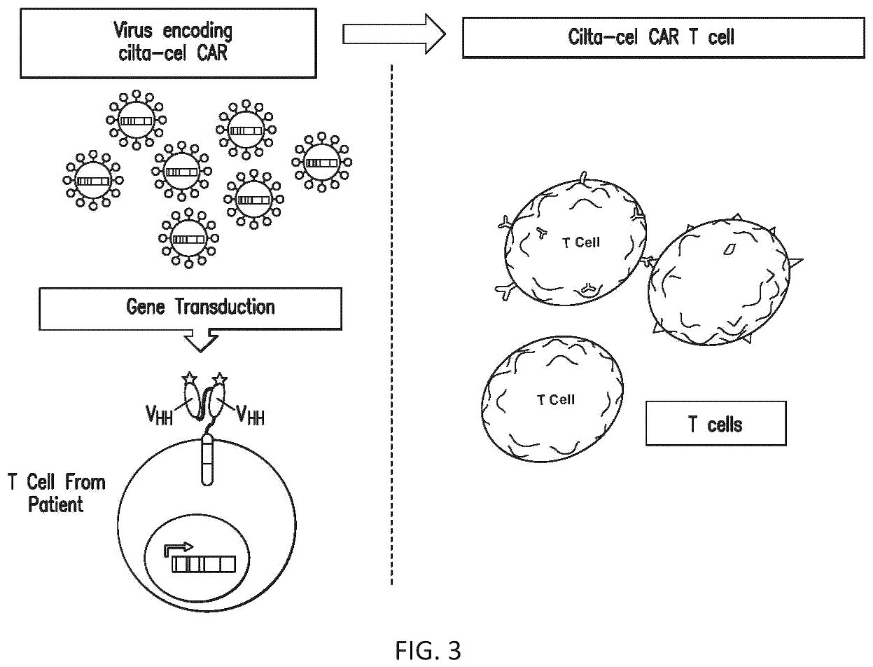

Ciltacabtagene autoleucel (cilta-cel) is an autologous chimeric antigen receptor T cell (CAR-T) therapy that targets BCMA. The ciltacabtagene autoleucel chimeric antigen receptor (CAR) comprises two B-cell maturation antigen (BCMA)-targeting VHH domains designed to confer avidity. A map of the construct is depicted in FIG. 2 and a schematic of the CAR-T cell production is shown in FIG. 3. Cilta-cel includes a VHH domain comprising the amino acid sequence set forth in SEQ ID NO: 2 and a VHH domain comprising the amino acid sequence set forth in SEQ ID NO: 4.

### Example 2: Method of Treatment with Ciltacabtagene Autoleucel

Cilta-cel is highly efficacious in heavily pretreated relapsed/refractory multiple myeloma (RRMM). In this study, we investigated cilta-cel in earlier treatment lines in lenalidomide-refractory patients.

Most patients with multiple myeloma (MM) relapse after standard treatment (van de Donk, Hematology Am Soc Hematol Educ Program 2020; 2020:248-58; Rodriguez-Lobato et al., Br J Haematol 2022; 196:649-59) and outcomes worsen with each subsequent line of therapy (LOT) (Yong et al., Br J Haematol 2016; 175:252-64; Dhakal et al., Clinical Lymphoma Myeloma and Leukemia 2022; 22:5167; Dhakal et al., HemaSphere 2022; 6:790-1). Lenalidomide is an immunomodulator recommended for newly diagnosed and relapsed/refractory MM (RRMM) (Dimopoulos et al., HemaSphere 2021; 5:e528; National Comprehensive Cancer Network. NCCN clinical practice guidelines in oncology, (NCCN Guidelines®) version 3.2023. 2023). Lenalidomide use has become widespread in early-line settings, including as maintenance therapy (van de Donk, Hematology Am Soc Hematol Educ Program 2020; 2020:248-58; de Arriba de la Fuente et al., Cancers (Basel) 2022;15). Rates of lenalidomide refractoriness early in patients' treatment journeys are increasing (van de Donk, Hematology Am Soc Hematol Educ Program 2020; 2020:248-58; de Arriba de la Fuente et al., Cancers (Basel) 2022;15), leading to a growing need for new, effective therapies for lenalidomide-refractory disease (de Arriba de la Fuente et al., Cancers (Basel) 2022;15). High treatment attrition-only 13% to 35% of patients receive 4 LOT—also highlights the need to use effective therapies early (Fonseca et al., BMC Cancer 2020; 20:1087).

Cilta-cel led to early, deep, and durable responses in patients with RRMM and >3 prior LOT in the phase 1b/2 CARTITUDE-1 trial (median progression-free survival [PFS], 34.9 months) (Berdeja et al., Lancet 2021; 398:314-24; Martin et al., J Clin Oncol 2022:JCO2200842; Lin et al., J Clin Oncol 2023. In submission). The phase 2 CARTITUDE-2 study (cohorts A and B) showed efficacy of cilta-cel in small cohorts at earlier disease stages, with response rates of 95%-100%, and median duration of response (DOR) and median PFS not reached after ˜1.5-year follow-up (van de Donk et al., Blood 2022; 140:7536-7; Einsele et al., American Society Of Clinical Oncology Annual Meeting; 2022, June 3-7; Chicago, IL).

CARTITUDE-4 is a phase 3, randomized, controlled trial comparing cilta-cel with physician's choice between two highly effective standard-of-care therapies in patients with lenalidomide-refractory MM after 1-3 LOT. We report efficacy and safety results from the first planned analysis of CARTITUDE-4.

### Study Design and Patients

CARTITUDE-4 is a global open-label, randomized, phase 3 trial conducted at 81 sites in the United States, Europe, Asia, and Australia. Eligible patients were lenalidomide refractory (Rajkumar et al., Blood 2011; 117:4691-5), had 1-3 prior LOT including a proteasome inhibitor and an immunomodulatory drug, an Eastern Cooperative Oncology Group performance status score of ≤1, no prior CAR-T therapy, and no previous BCMA-targeted treatment.

### Randomization and Treatments

Patients were assigned 1:1 via computer-generated randomization to receive standard-of-care (physicians' choice of pomalidomide-bortezomib-dexamethasone [PVd] (Richardson et al., Lancet Oncol 2019; 20:781-94) or daratumumab-pomalidomide-dexamethasone [DPd]) (Dimopoulos et al., Lancet Oncol 2021; 22:801-12) or a single cilta-cel infusion following physicians' choice of bridging therapy (PVd or DPd). Randomization was stratified by choice of PVd vs. DPd, International Staging System (ISS) stage at screening (I vs. II vs. III), and number of prior LOT (1 vs. 2-3).

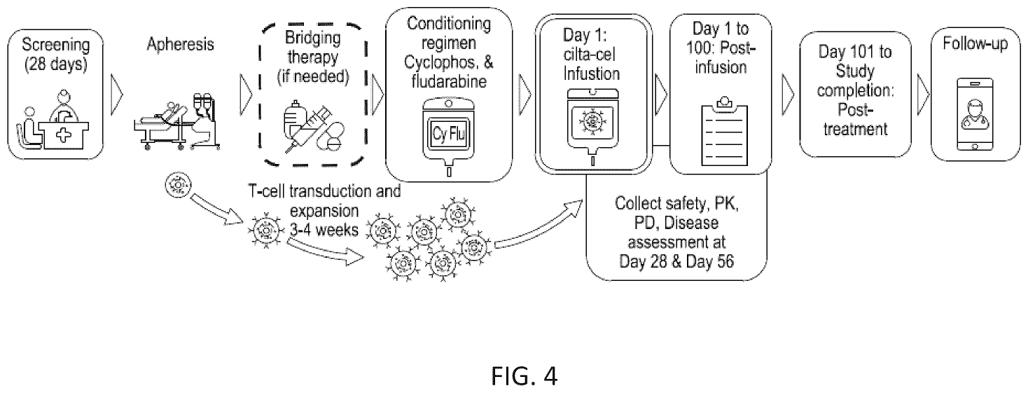

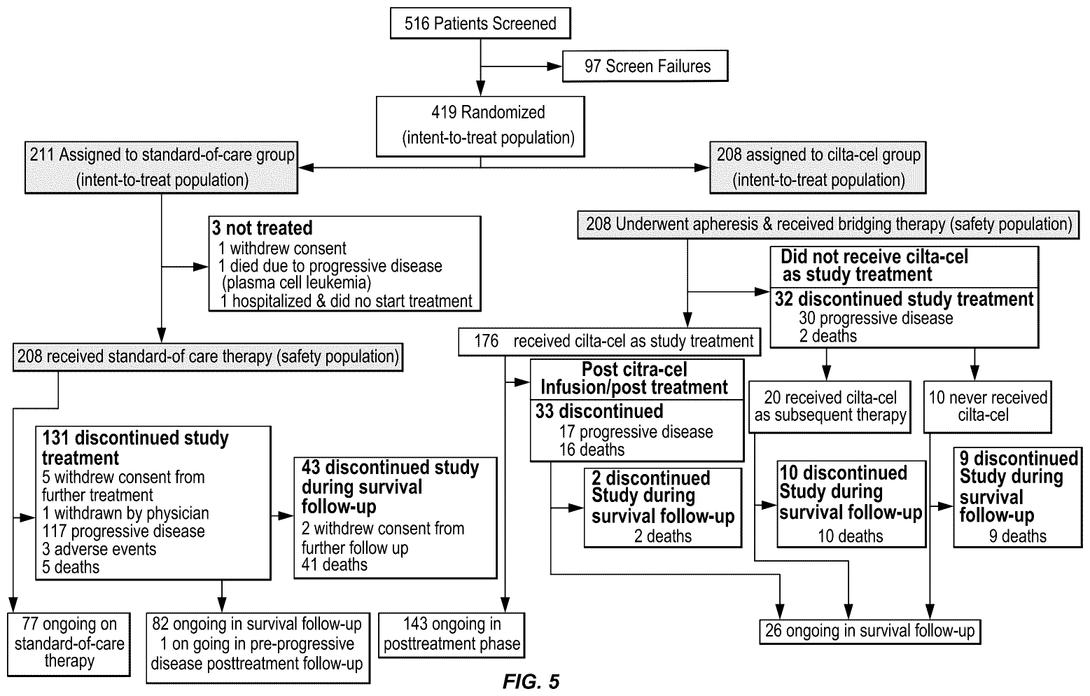

In the standard-of-care arm, DPd was administered in 28-day cycles and PVd in 21-day cycles until disease progression. Patients in the cilta-cel arm underwent apheresis, followed by ≥1 bridging therapy cycles (number of cycles based on clinical status and cilta-cel manufacturing time) and lymphodepletion (300 mg/m² cyclophosphamide and 30 mg/m² fludarabine daily for 3 days). A single cilta-cel infusion (target dose, 0.75×10⁶ CAR+ viable T cells/kg) was administered 5-7 days after start of lymphodepletion (FIG. 4). Patients randomized to the cilta-cel arm who had confirmed disease progression during bridging therapy or lymphodepletion were assessed as having a PFS event and could receive cilta-cel as subsequent therapy, at investigator discretion.

### Standard-of-Care and Bridging Therapy Cycles

DPd as standard care was administered in 28-day cycles comprising 1800 mg subcutaneous daratumumab (days 1, 8, 15, and 22 of cycles 1 and 2; days 1 and 15 of cycles 3 to 6; and day 1 of each cycle thereafter), 4 mg/day of oral pomalidomide on days 1 to 21, and oral or intravenous dexamethasone in 40-mg weekly doses on days 1, 8, 15, and 22 or split over 2 days. PVd as standard care was administered in 21-day cycles of 4 mg oral pomalidomide daily on days 1 to 14; 1.3 mg/m² subcutaneous bortezomib on days 1, 4, 8, and 11 of cycles 1 to 8 and on days 1 and 8 of each cycle thereafter; and 20 mg/day of oral dexamethasone on days 1, 2, 4, 5, 8, 9, 11, and day 12 of cycles 1 to 8 and on days 1, 2, 8, and 9 of each cycle thereafter.

DPd as bridging therapy was administered in 28-day cycles comprising 1800 mg subcutaneous daratumumab on bridging days 1, 8, 15, and 22; 4 mg/day of oral pomalidomide for 21 days; and 40 mg of oral or intravenous dexamethasone on bridging days 1, 8, 15, and 22 or split in two 20-mg doses over 2 days. PVd as bridging therapy was administered in 21-day cycles comprising 4 mg/day of oral pomalidomide for 14 days; 1.3 mg/m² of subcutaneous bortezomib on bridging days 1, 4, 8, and 11; and 20 mg of oral dexamethasone on bridging days 1, 2, 4, 5, 8, 9, 11, and 12.

### Cilta-Cel Pharmacokinetics

The median time from first apheresis to cilta-cel infusion was 79.0 days (range, 45-246). Reasons for this included COVID-19 associated delays.

CD3+CAR+ cells in blood peaked at median 13 days post infusion (mean, 1523 cells/μL; SD, 5987 cells/μL) and remained detectable for median 57 days (range, 13-631). Mean AUC_{0-28d} of CD3+CAR+ cells in blood was 12,504 cells/μL (SD, 55,281 cells/μL).

### Endpoints and Assessments

The primary endpoint was PFS, defined as time from randomization to first documented disease progression/death due to any cause. Secondary endpoints included rates of complete response (CR) or better, overall response, minimal residual disease (MRD) negativity, overall survival (OS), time to patient-reported symptom worsening as assessed by the MM Symptom and Impact Questionnaire, adverse events (AEs), and cilta-cel pharmacokinetics.

Treatment responses and disease progression were determined per International Myeloma Working Group criteria (Rajkumar et al., Blood 2011;117:4691-5) using a validated computer algorithm (Palumbo, et al., N Engl J Med 2016; 375:754-66). Blood and 24-hour urine samples were analyzed at a central laboratory until confirmed disease progression. MRD (10⁻⁵ sensitivity) was assessed centrally by next-generation sequencing (clonoSEQ v2.0; Adaptive Biotechnologies, Seattle, WA) on bone marrow samples.

Cytokine release syndrome (CRS) and immune effector cell-associated neurotoxicity syndrome (ICANS) were graded per American Society for Transplantation and Cellular Therapy consensus grading (Lee et al., Biol Blood Marrow Transplant 2019; 25:625-38). Other AEs, including investigator-assessed non-ICANS neurotoxicity, were graded per National Cancer Institute Common Terminology Criteria for Adverse Events (NCI-CTCAE), version 5.0 (Xu et al., Stat Med 2017; 36:592-605).

### Trial Oversight

This study was conducted in accordance with the Declaration of Helsinki and International Council for Harmonisation guidelines for Good Clinical Practice. All patients provided written informed consent. An independent ethics committee or institutional review board at each site approved the study protocol. A data monitoring committee was established to monitor safety data collected in the clinical program and to evaluate the interim safety and efficacy data (Supplementary Appendix). All authors contributed to study conduct, data analyses, and drafting of the manuscript and vouch for the work. The authors assure data accuracy and completeness and adherence to the trial protocol.

### Statistical Analysis

We estimated 400 patients and 250 PFS events would achieve 90% power to detect a 35% reduction in risk of disease progression/death with a log-rank test at an overall two-sided alpha level of 0.05, under a group sequential design with one interim analysis to evaluate the primary endpoint, prespecified to be conducted after approximately 188 PFS events. The significance level required to establish superiority was determined based on the number of events observed, using O'Brien-Fleming boundaries, implemented by the Lan-DeMets alpha spending method. Based on 187 PFS events observed at the interim analysis, the two-sided alpha to be spent in the interim analysis is 0.0191. If the observed two-sided p-value is smaller than 0.0191, the superiority of cilta-cel over DPd or PVd with respect to PFS will be established.

### Timing of Assessments for Disease Progression and Minimal Residual Disease

In the standard-of-care arm, tests were performed on the first day of each treatment cycle, then every 28 days until confirmed disease progression or the start of subsequent therapy, and at the end-of-treatment visit, whichever came first. In the cilta-cel arm, assessments were conducted at or ≤3 days before apheresis, on the first day of each bridging therapy cycle starting at cycle 2, ≤7 days before lymphodepletion initiation, and every 4 weeks starting 28 days after cilta-cel infusion. For minimal residual disease assessments in both arms, samples were collected at screening for baseline clone identification, at time of suspected complete response/stringent complete response, at 6, 12, 18, and 24 months from day 1 of study treatment or from cilta-cel infusion, and then yearly thereafter until disease progression for patients in complete response/stringent complete response at 24 months. In the cilta-cel arm, an additional assessment was made at day 56 post cilta-cel infusion.

### Timing of Patient-Reported Outcomes Assessments

Questionnaires were completed on day 1 of cycles 1 to 5, 9, 13, 17, and every 8 cycles thereafter until disease progression in patients who received PVd as standard-of-care; day 1 of cycles 1—to 4, 7, 10, 13, and every 6 cycles thereafter until disease progression in patients who received DPd as standard-of-care; and within 72 hours of apheresis, on day 1 of bridging therapy cycle 1, on the first day of lymphodepletion prior to lymphodepletion start, on post-cilta-cel infusion days 28, 112, 196, and 280, every 24 weeks thereafter, and every 16 weeks after disease progression.

### Pharmacokinetics

CAR+ T cell levels in blood were evaluated for pharmacokinetics in the cilta-cel arm using samples collected on day 1 pre-infusion, and then on days 3, 7, 10, 14, 28, 56, 84, and 112, every 8 weeks for up to 1 year starting on day 140, and at disease progression or end of study.

Pharmacokinetic and Correlative Analysis of Ciltacabtagene Autoleucel in Patients with Lenalidomide-Refractory Multiple Myeloma in the CARTITUDE-4 Trial

As of November 2022, among the 176 pts who received cilta-cel as study treatment, CD3+CAR+ cells in blood peaked at median 13 d post infusion (mean, 1523 cells/μL; SD, 5987 cells/μL) and remained detectable for median 57 d (range, 13-631). Mean AUC0-28d of CD3+CAR+ cells in blood was 12,504 cells/μL (SD, 55,281 cells/μL). In 13 pts with PD and available data, we did not observe a reappearance or re-expansion of CD3+CAR+ cells in blood at PD; CAR+ T cells were below the lower limit of quantification (2 cells/mL) in all pts. Serum BCMA (sBCMA) results were available for 3 pts and proportion of BCMA+MM cells in the BMA were available for 6, at PD. After an initial decline following cilta-cel infusion, sBCMA levels either returned to or exceeded baseline levels at PD. Similar findings were observed in the 6 pts with available data on the proportion of BCMA+MM cells in the bone marrow. Consistent with observations in CARTITUDE-1, CAR-T cell expansion or persistence were not correlated with response, and responses extended beyond the median duration of CAR-T cells detectability. Based on the few samples available at data cut off, BCMA levels return to levels similar to or above baseline at time of relapse, without reappearance or re-expansion of CD3+CAR+ cells in blood. Understanding mechanisms of resistance is an area of interest which will ultimately assist with the sequencing of MM therapies.

### Grading of Adverse Events Related to Cytokine Release Syndrome and Immune Effector Cell-Associated Neurotoxicity

American Society for Transplantation and Cellular Therapy consensus grading was used to grade cytokine release syndrome and immune effector cell-associated neurotoxicity syndrome; however, symptoms of immune effector cell-associated neurotoxicity and symptoms of cytokine release syndrome were graded per National Cancer Institute Common Terminology Criteria for Adverse Events (NCI-CTCAE), version 50.

### COVID-19 Safety Measures

Measures to prevent and mitigate risk of COVID-19 infection were introduced during the study, including education on the importance of re-vaccination after cilta-cel, and other preventative measures, and investigators were asked to consider the use of prophylaxis and antiviral therapies such as Evusheld, (tixagevimab/cilgavimab), and early use of Paxlovid (nirmatrelvir/ritonavir) where available. Sites were asked to follow recommendations from the American Society of Hematology and American Society for Transplantation and Cellular Therapy (ASH-ASTCT COVID-19 Vaccination for Hematopoietic Cell Transplant and CAR-T Cell Recipients: Frequently Asked Questions) and the European Society for Blood and Marrow Transplantation (EMBT) (Coronavirus Disease COVID-19: EBMT Recommendations).

### Data Monitoring Committee

A data monitoring committee (consisting of 2 clinicians and 1 statistician) was established to review efficacy and safety results at the planned interim analysis for the primary efficacy endpoint.

Members of the data monitoring committee included Dr. Heinz Ludwig (Chair, Wilhelminen Cancer Research Institute), Dr. Dianne Finkelstein (Massachusetts General Hospital Biostatistics Center), and Dr. Adam Cohen (University of Pennsylvania).

Efficacy was evaluated in the intent-to-treat (ITT) population (all randomized patients) and in patients who received cilta-cel as study treatment. AEs were evaluated in the safety population (all patients who received any part of study treatment: standard-of-care arm: any DPd/PVd component; cilta-cel arm: apheresis, bridging therapy, lymphodepletion, or cilta-cel). AEs specific to CAR-T therapy were evaluated in patients who received cilta-cel as study treatment.

PFS was estimated using the Kaplan-Meier method. A stratified constant piecewise weighted log-rank test (assigning a weight of 0 for the log-rank statistic for weeks 0-8 post randomization, and 1 afterwards) (Zucker et al., Biometrika 1990; 77:853-64; Rodriguez-Otero et al., N Engl J Med 2023) was used to compare the cilta-cel and standard-of-care arms, as both received the same treatments during the bridging period. The hazard ratio (HR) and its two-sided 95% confidence intervals (CIs) were estimated using a stratified Cox regression model with treatment as the sole explanatory variable. The Kaplan-Meier method and stratified log-rank tests were used to analyze other time-to-event endpoints. Binary endpoints were analyzed using stratified Cochran-Mantel-Haenszel tests. Time to symptom worsening was defined as a meaningful decline (estimated by distribution-based methods of at least half a standard deviation [SD] of pooled baseline values) without subsequent improvement of MM symptoms.

### Patients

Between Jul. 10, 2020, and Nov. 17, 2021, 419 patients were randomized to cilta-cel (n=208) or standard-of-care (n=211; DPd [n=183] or PVd [n=28]) arms. All patients in the cilta-cel arm received bridging therapy (DPd [n=182] or PVd [n=26]). 176 (84.6%) received cilta-cel as study treatment. 32 patients discontinued study treatment before receiving cilta-cel, predominantly due to disease progression during bridging therapy/lymphodepletion. Of these 32 patients, 20 received cilta-cel as subsequent therapy. No patients discontinued study treatment due to manufacturing failure. Median time from apheresis material receipt to product release was 44 days (range, 25-127). 208 (98.6%) standard-of-care arm patients were dosed and 131 (63%) discontinued treatment, primarily due to disease progression (56.3%) (FIG. 5). At data cut-off (Nov. 1, 2022) median follow-up was 15.9 months (range, 0.1-27.3).

Patient characteristics were well balanced between arms (Table 1); patient demographics largely reflected real-world patients with myeloma (Table 2). 59.4% of cilta-cel and 62.9% of standard-of-care arm patients had high-risk cytogenetics (del(17p), t(4:14), t(14;16), or gain/amp(1q)); 20.7% and 23.2% had ≥2 high-risk abnormalities; 21.2% and 16.6% had soft tissue plasmacytomas at baseline. 30 (14.4%) cilta-cel arm patients were triple-class refractory; 50 (24.0%) were anti-CD38 antibody refractory. Median cilta-cel dose was 0.71×10⁶ cells/kg, and standard-of-care arm patients received a median 12 (range, 1-28) treatment cycles.

### Example 3: Evaluation of Efficacy of Method of Treatment with Ciltacabtagene Autoleucel

Using the IMWG-based response criteria summarized in Table 3, this study classified a response, in order from better to worse, as either a stringent complete response (sCR), a complete response (CR), a very good partial response (VGPR), a partial response (PR), a minimal response (MR), a stable disease or a progressive disease. Disease progression was consistently documented across clinical study sites. The tests performed to assess IMWG-based response criteria are as follows:
Myeloma Protein Measurements in Serum and Urine: Myeloma protein (M-protein) measurements were made using the following tests from blood and 24-hour urine samples: serum quantitative Ig, serum protein electrophoresis (SPEP), serum immunofixation electrophoresis, serum FLC assay (for subject in suspected CR/sCR and every disease assessment for subjects with serum FLC only disease), 24-hour urine M-protein quantitation by electrophoresis (UPEP), urine immunofixation electrophoresis, serum 02-microglobulin. Disease progression based on one of the laboratory tests alone were confirmed by at least 1 repeat investigation. Disease evaluations continued beyond relapse from CR until disease progression was confirmed. Serum and urine immunofixation and serum free light chain (FLC) assays were performed at screening and thereafter when a CR was suspected (when serum or 24-hour urine M-protein electrophoresis [by SPEP or UPEP] were 0 or non-quantifiable). For subjects with light chain multiple myeloma, serum and urine immunofixation tests were performed routinely.
Serum Calcium Corrected for Albumin: Blood samples for calculating serum calcium corrected for albumin were collected and analyzed until the development of confirmed disease progression; development of hypercalcemia (corrected serum calcium >11.5 mg/dL [>2.9 mmol/L]) may indicate disease progression or relapse if it is not attributable to any other cause. Calcium binds to albumin and only the unbound (free) calcium is biologically active; therefore, the serum calcium level must be adjusted for abnormal albumin levels (“corrected serum calcium”).
Bone Marrow Examination: Bone marrow aspirate or biopsy was performed for clinical assessments. Bone marrow aspirate was performed for biomarker evaluations. Clinical staging (morphology, cytogenetics, and immunohistochemistry or immunofluorescence or flow cytometry) was done. A portion of the bone marrow aspirate was immunophenotyped and monitor for BCMA, checkpoint ligand expression in CD138-positive multiple myeloma cells, and checkpoint expression on T cells. If feasible, bone marrow aspirate also was performed to confirm CR and sCR and at disease progression. Additionally, since minimal residual disease (MRD) negativity was being evaluated as a potential surrogate for PFS and OS in multiple myeloma treatment, MRD was monitored in subjects using next generation sequencing (NGS) on bone marrow aspirate DNA. Baseline bone marrow aspirates were used to define the myeloma clones, and post-treatment samples were used to evaluate MRD negativity. A fresh bone marrow aspirate was collected prior to the first dose of conditioning regimen (≤7 days).
Skeletal Survey: A skeletal survey (including skull, entire vertebral column, pelvis, chest, humeri, femora, and any other bones for which the investigator suspects involvement by disease) was performed during the screening phase and evaluated by either roentgenography (X-rays) or low-dose computed tomography (CT) scans without the use of IV contrast. If a CT scan was used, it was of diagnostic quality. Following cilta-cel infusion, and before disease progression was confirmed, X-rays or CT scans were performed locally, whenever clinically indicated based on symptoms, to document response or progression. Magnetic resonance imaging (MRI) was an acceptable method for evaluation of bone disease, and was included at discretion; however, it did not replace the skeletal survey. If a radionuclide bone scan was used at screening, in addition to the complete skeletal survey, then both methods were used to document disease status. These tests were performed at the same time. A radionuclide bone scan did not replace a complete skeletal survey. If a subject presented with disease progression manifested by symptoms of pain due to bone changes, then disease progression was documented by skeletal survey or other radiographs, depending on the symptoms that the subject experiences. If the diagnosis of disease progression was obvious by radiographic investigations, then no repeat confirmatory X-rays were thought necessary to perform. If changes were equivocal, then a repeat X-ray was performed in 1 to 3 weeks.
Documentation of Extramedullary Plasmacytomas: Sites of known extramedullary plasmacytomas were documented ≤14 days prior to the first dose of the conditioning regimen. Clinical examination or MRI were used to document extramedullary sites of disease. CT scan evaluations were considered an acceptable alternative if there was no contraindication to the use of IV contrast. Positron emission tomography scan or ultrasound tests were not acceptable to document the size of extramedullary plasmacytomas. However, PET/CT fusion scans were optionally used to document extramedullary plasmacytomas if the CT component of the PET/CT fusion scan was of sufficient diagnostic quality. Extramedullary plasmacytomas were assessed for all subjects with a history of plasmacytomas or if clinically indicated at ≤14 days prior to the first dose of the conditioning regimen, by clinical examination or radiologic imaging. Assessment of measurable sites of extramedullary disease were performed, measured, and evaluated locally every 4 weeks (for physical examination) for subjects with a history of plasmacytomas or as clinically indicated during treatment for other subjects until development of confirmed CR or confirmed disease progression. If assessment could only be performed radiologically, then evaluation of extramedullary plasmacytomas was done every 12 weeks. Irradiated or excised lesions were considered not measurable and were monitored only for disease progression. To qualify for VGPR or PR/minimal response (MR), the sum of products of the perpendicular diameters of the existing extramedullary plasmacytomas must have decreased by over 90% or at least 50%, respectively, and new plasmacytomas must not have developed. To qualify for disease progression, either the sum of products of the perpendicular diameters of the existing extramedullary plasmacytomas must have increased by at least 50%, or the longest diameter of previous lesion >1 cm in short axis must have increased at least 50%, or a new plasmacytoma must have developed. When not all existing extramedullary plasmacytomas were reported, but the sum of products of the perpendicular diameters of the reported plasmacytomas had increased by at least 50%, then the criterion for disease progression was met.

If it was determined that the study treatment interfered with the immunofixation assay, CR was defined as the disappearance of the original M-protein associated with multiple myeloma on immunofixation, and the determination of CR was not affected by unrelated M-proteins secondary to the study treatment.

Study endpoints, as assessed by an independent review committee (IRC), were as follows:
MRD was assessed at baseline, day 28, and 6-, 12-, 18-, and 24-month follow-ups using next-generation sequencing (clonoSEQ version 2.0) (Adaptive Biotechnologies, Seattle, WA, USA) in patients at the time of suspected complete response, and then every 12 months until disease progression for patients who remained on study. MRD negativity was assessed in samples that passed calibration or quality control and included sufficient cells for evaluation at the testing threshold of 10⁻⁵. Durability of MRD-negative status was evaluated by estimating MRD negativity rates at 6- and 12-month follow-ups.
Clinical benefit rate (CBR) was defined as the proportion of subjects who achieved a MR or better according to the IMWG criteria (sCR+CR+VGPR+PR+MR).
Overall response rate (ORR) was defined as the proportion of subjects who achieved a PR or better according to the IMWG criteria (sCR+CR+VGPR+PR).
VGPR or better response rate was defined as the proportion of subjects who achieve a VGPR or better response according to the IMWG criteria (sCR+CR+VGPR).
Duration of response (DOR) was calculated among responders (with a PR or better response) from the date of initial documentation of a response (PR or better) to the date of first documented evidence of progressive disease, as defined in the IMWG criteria. Relapse from CR by positive immunofixation or trace amount of M-protein was not considered as disease progression. Disease evaluations continued beyond relapse from CR until disease progression was confirmed.
Time to response (TTR) was defined as the time between date of the initial infusion of cilta-cel and the first efficacy evaluation at which the subject had met all criteria for PR or better.
Progression-free survival (PFS) was defined as the time from the date of the initial infusion of cilta-cel to the date of first documented disease progression, as defined in the IMWG criteria, or death due to any cause, whichever occurred first.
Overall survival (OS) was measured from the date of the initial infusion of cilta-cel to the date of the subject's death.

For ORR, the response rate and its 95% exact confidence interval (CI) was calculated based on binomial distribution, and the null hypothesis was rejected if the lower bound of the confidence interval exceeded 30%. Analysis of VGPR or better response rate, DOR, PFS, and OS was conducted at the same cutoff as the ORR. Time-to-event efficacy endpoints (DOR, PFS, and OS) were estimated using the Kaplan-Meier method. The distribution (median and Kaplan-Meier curves) of DOR was provided using Kaplan-Meier estimates. Similar analysis was performed for OS, PFS, and TTR.

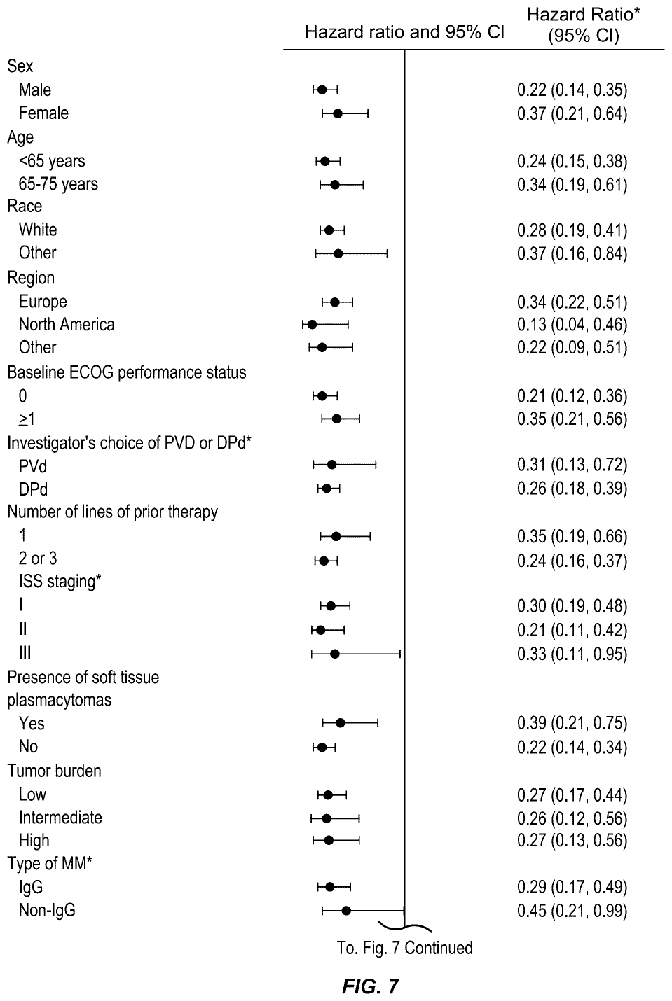

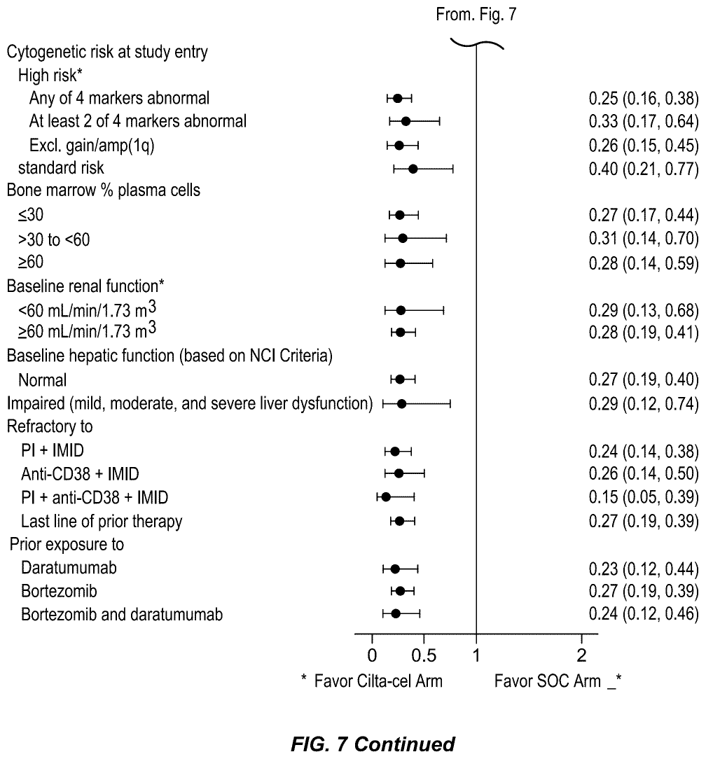

CARTITUDE-4 met its primary endpoint. In the ITT population, cilta-cel significantly reduced risk of disease progression/death vs. standard-of-care (HR, 0.26; 95% CI, 0.18-0.38; P<0.0001). Median PFS was not reached (95% CI, 22.8—not estimable [NE]) in the cilta-cel arm vs. 11.8 months (95% CI, 9.7-13.8) in the standard-of-care arm (FIG. 6A). An unweighted sensitivity analysis showed similar results (Table 4). 12-month PFS rates (95% CI) were 75.9% (69.4-81.1) and 48.6% (41.5-55.3), respectively. Cilta-cel prolonged PFS vs. standard-of-care in all subgroups, including those with high-risk cytogenetics, soft tissue plasmacytomas, triple-class-refractory disease, and other high-risk disease factors, and across different numbers of prior LOT (FIG. 6B, FIG. 7). During the first eight weeks after randomization, there were 22 PFS events in the cilta-cel arm and 8 in the standard-of-care arm. All these events occurred before cilta-cel infusion while patients were on bridging therapy (same treatment in both arms). This imbalance may have been due to approximately 14% lower relative doses of pomalidomide and bortezomib during bridging therapy in the cilta-cel vs. standard-of-care arm in the same period.

In the ITT population, overall response rate (partial response or better) was 84.6% with cilta-cel vs. 67.3% with standard-of-care (RR, 2.2 [95% CI, 1.5-3.1]; odds ratio [OR], 3.0; P<0.0001), and more patients achieved ≥CR with cilta-cel (73.1% vs. 21.8%; risk ratio [RR], 2.9 [95% CI, 2.3-3.7]; OR, 10.3; P<0.0001) (Table 5). Median DOR was not reached in the cilta-cel arm vs. 16.6 months in the standard-of-care arm. An estimated 84.7% of responders in the cilta-cel arm remained in response for at least 12 months vs. 63.0% in the standard care arm.

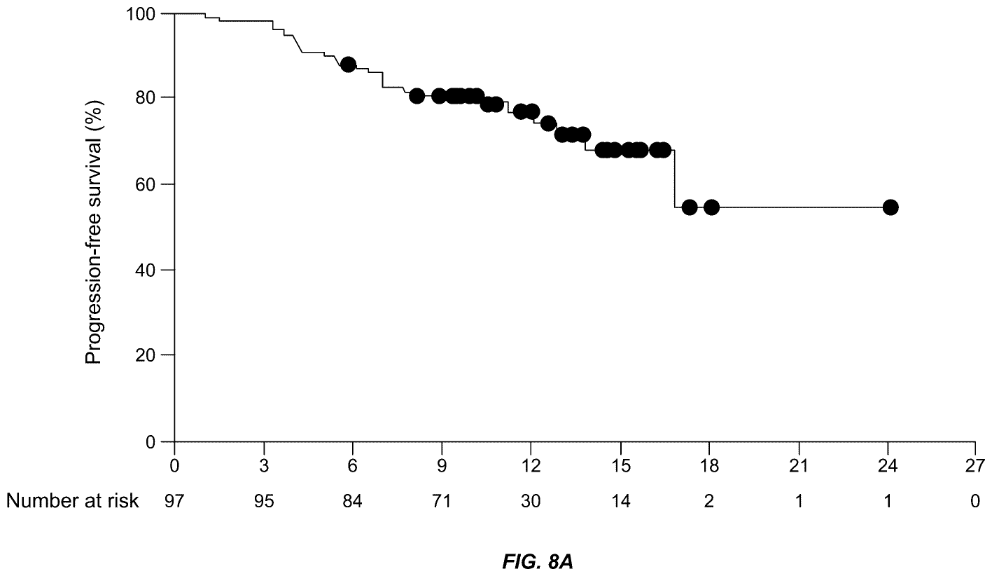

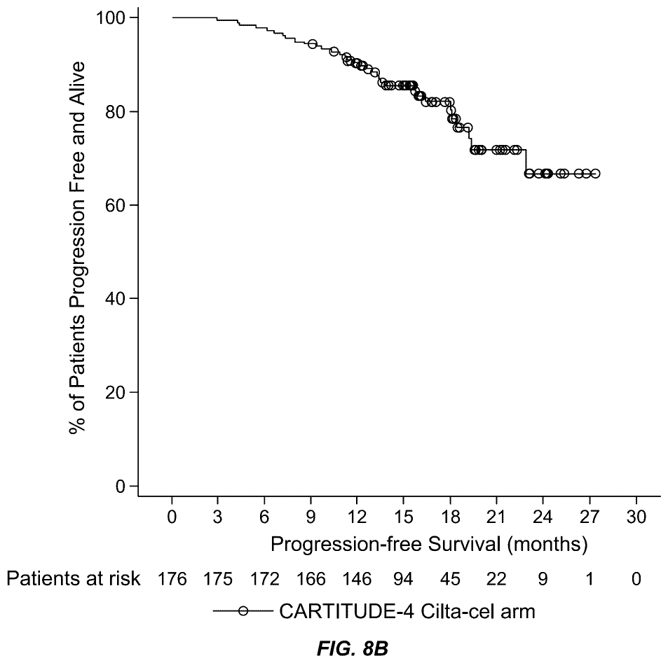

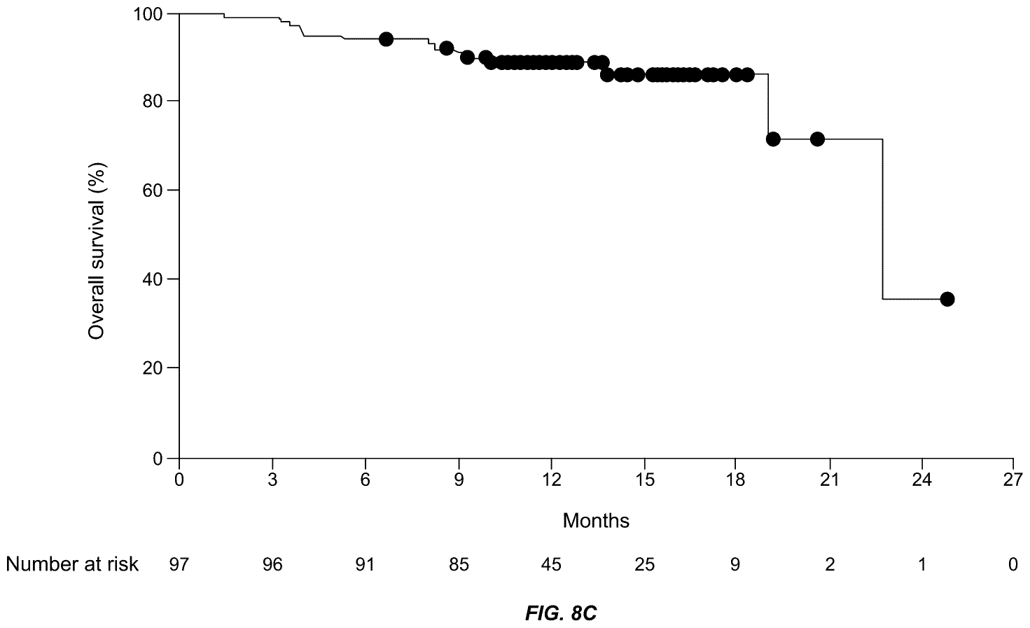

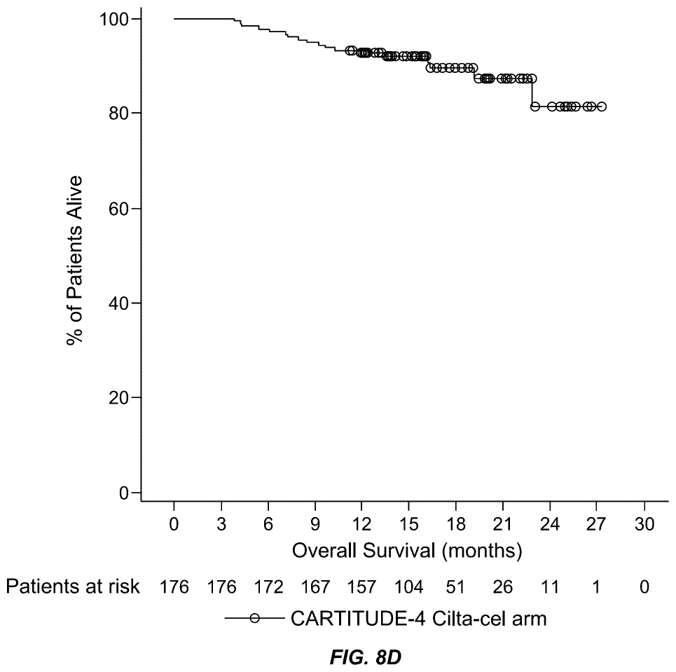

Focusing on patients who received cilta-cel as study treatment (n=176/208), 99.4% responded and 86.4% achieved ≥CR (Table 5). The 12-month PFS rate was 89.7% (PFS curve shown in FIGS. 8A-8D).

In the cilta-cel and standard-of-care arms (ITT), 60.6% and 15.6% of patients, respectively, achieved MRD negativity at any time up to data cut-off during the study (RR, 2.2, 95% CI, 1.8-2.6; OR, 8.7; P<0.0001; Table 5). Among patients with evaluable samples (cilta-cel [n=144]; standard-of-care [n=101]), 126 (87.5%) and 33 (32.7%) achieved MRD negativity.

OS data were immature; median OS was not reached in the cilta-cel arm (39 deaths) vs. an estimated 26.7 months in the standard-of-care arm (47 deaths; HR, 0.78; 95% CI, 0.5-1.2, P=0.26; FIG. 6C, FIGS. 8A-8D).

Median times to symptom worsening were 23.7 months (95% CI, 22.1—NE) in the cilta-cel arm and 18.9 months (95% CI, 16.8—NE) in the standard-of-care arm (HR, 0.42, 95% CI, 0.26-0.68; nominal P=0.0003).

In patients with lenalidomide-refractory MM after 1-3 prior LOT, a single cilta-cel infusion demonstrated superior efficacy vs. highly effective standard-of-care regimens (mostly DPd) at 15.9-month median follow-up. Cilta-cel reduced risk of disease progression or death by 74%; 12-month PFS rate was 76% vs. 49% with standard-of-care. Of note, the 12-month PFS rate increased to 90% in patients who received cilta-cel as study treatment. The efficacy benefit was apparent across all subgroups analyzed, including patients with high-risk cytogenetic abnormalities, soft tissue plasmacytomas, triple-class-refractory disease, ISS stage III status, and other high-risk features. Cilta-cel demonstrated higher response rates, deeper and more durable responses, and higher MRD negativity rates than standard-of-care, with delayed patient-reported symptom worsening. These results demonstrate that cilta-cel is an effective treatment for patients with lenalidomide-refractory disease as early as first relapse; add to the consistently strong efficacy cilta-cel has shown throughout its clinical development, including in similar, early LOT populations in CARTITUDE-2 (Van De Donk et al., Blood 2022; 140:7536-7; Einsele et al., American Society Of Clinical Oncology Annual Meeting; 2022 June 3-7; Chicago, IL.); and confirm the profound efficacy impact observed in heavily pretreated patients who received cilta-cel in CARTITUDE-1 (Fonseca, et al., BMC Cancer 2020; 20:1087; Berdeja et al., Lancet 2021; 398:314-24).

All subgroup analyses of PFS in the cilta-cel vs standard care groups—including in patients with 1 prior line of therapy, high-risk cytogenetic abnormalities at baseline, soft-tissue plasmacytomas at baseline, and triple-class-refractory disease—showed effects similar to that observed in the analysis of the ITT population (FIG. 7).

Acknowledging the differences in study designs, particularly patient populations, and limitations of cross-trial comparisons, the 12-month PFS rate in the cilta-cel arm (76%) compares favorably with that of idecabtagene vicleucel (ide-cel) among patients with RRMM and 2-4 prior LOT in the KarMMa-3 study (55%), the only other head-to-head, phase 3 CAR-T therapy study for MM (Rodriguez-Otero et al., N Engl J Med 2023). Cilta-cel also had a favorable HR of 0.26 vs. standard-of-care (which performed as expected in CARTITUDE-4) compared with the HR of 0.49 for ide-cel vs. standard-of-care (Cohen et al., Blood 2022).

Patients in both arms of CARTITUDE-4 received the same medications during bridging therapy; therefore, a prespecified weighting methodology was applied to both arms to focus outcomes on events after cilta-cel infusion. The higher number of PFS events reported during study weeks 0-8 in the cilta-cel vs. standard-of-care arms, all of which preceded cilta-cel infusion, may have been due to lower DPd/PVd dose intensities in the cilta-cel arm. These early events resulted in the benefit of cilta-cel only becoming apparent in the PFS Kaplan-Meier curve at 3 months.

In an unweighted sensitivity analysis, cilta-cel improved progression-free survival compared with standard care (HR, 0.4; 95% CI, 0.29-0.55). Results were similar to those of the protocol-specified stratified constant piecewise weighted log-rank test showing an HR of 0.26 (95% CI, 0.18-0.38).

Because the protocol-specified efficacy analyses were performed in the intent-to-treat population, those results include patients who did not receive cilta-cel. To highlight the clinical benefit that a single cilta-cel infusion can provide to lenalidomide refractory patients with 1-3 prior lines of therapy, we examined efficacy outcomes in patients who received cilta-cel as study treatment (n=176 of 208 randomized).

At data cutoff, median PFS was not estimable in patients who received cilta-cel as study treatment, and the 12-month PFS rate was 89.7% (FIGS. 8A-8D). Additionally, the overall response rate in this population was 99.4%, including 86.4% of patients who achieved ≥CR (sCR, 68.8%; CR, 17.6%); and median duration of response was not estimable. Similarly, the minimal residual disease (MRD) negativity (10-5 sensitivity) rate among patients who received cilta-cel as study treatment was high (n/N, 126/176 [71.6%]) (Table 15).

### Example 4: Evaluation of Safety of Method of Treatment with Ciltacabtagene Autoleucel

Adverse events were followed, reported and graded according to the National Cancer Institute Common Terminology Criteria for Adverse Events (NCI-CTCAE Version 5.0), with the exception of CRS and CAR-T cell-related neurotoxicity (e.g., ICANS). CRS was evaluated according to the ASTCT consensus grading, summarized in Table 6. At the first sign of CRS (such as fever), subjects were immediately hospitalized for evaluation. Tocilizumab intervention was discretionally used to treat subjects presenting symptoms of fever when other sources of fever had been eliminated. Tocilizumab was discretionally used for early treatment in subjects at high risk of severe CRS (for example, high baseline tumor burden, early fever onset, or persistent fever after 24 hours of symptomatic treatment). Other monoclonal antibodies targeting cytokines (for example, anti-IL1 and/or anti-TNFα) were optionally used, especially for cases of CRS which did not respond to tocilizumab.

CAR-T cell-related neurotoxicity (e.g., ICANS) was graded using the ASTCT consensus grading, summarized in Table 7 Additionally, all individual symptoms of CRS (e.g., fever, hypotension) and ICANS (e.g., depressed level of consciousness, seizures) were captured as individual adverse events and graded by CTCAE criteria. Neurotoxicity that was not temporarily associated with CRS, or any other neurologic adverse events that did not qualify as ICANS, were graded by CTCAE criteria. Any adverse event or serious adverse event not listed in the NCI CTCAE Version 5.0 was graded according to investigator clinical judgment by using the standard grades as follows:
Grade 1: Mild; asymptomatic or mild symptoms; clinical or diagnostic observations only; intervention not indicated.
Grade 2: Moderate; minimal, local or noninvasive intervention indicated; limiting age-appropriate instrumental activities of daily living.
Grade 3: Severe or medically significant but not immediately life-threatening; hospitalization or prolongation of hospitalization indicated; disabling; limiting self-care activities of daily living.
Grade 4: Life-threatening consequences; urgent intervention indicated.
Grade 5: Death related to adverse event.

In the safety population (cilta-cel [n=208]; standard-of-care [n=208]), grade 3/4 treatment-emergent AEs (TEAEs) occurred in 201 (96.6%) cilta-cel and 196 (94.2%) standard-of-care arm patients (Table 8). The most common grade 3/4 AEs in both arms were hematologic, and most high-grade cytopenias in patients who received cilta-cel as study treatment recovered to grade ≤2 by day 60 (Table 9 and Table 16). Treatment-emergent serious AEs were reported in 92 (44.2%) cilta-cel and 81 (38.9%) standard-of-care arm patients. In the standard-of-care arm, 3 (1.4%) patients discontinued treatment and 115 (55.3%) had cycle delays due to AEs.

Nine (4.3%) cilta-cel and 14 (6.7%) standard-of-care arm patients had second primary malignancies; hematologic and cutaneous/noninvasive malignancies were the most common (Table 10).

Treatment-emergent infections occurred in 129 cilta-cel (62.0%; 26.9% grade 3/4) and 148 (71.2%; 24.5% grade 3/4) standard-of-care arm patients. Respectively, 69 (33.2%) and 56 (26.9%) had COVID-19 infection, with 29 (13.9%) and 55 (26.4%) considered treatment emergent (Table 8). Incidences of treatment-emergent hypogammaglobulinemia (based on AE reporting and laboratory results) were 90.9% and 71.6%; 65.9% and 12.5% of patients received intravenous immunoglobulin.

39 cilta-cel and 46 standard-of-care arm patients in the safety population died (1 additional patient in the standard-of-care ITT population died prior to treatment). 14 cilta-cel (8 of whom never received cilta-cel) and 30 standard-of-care arm patients died of disease progression; 10 and 5 deaths were due to TEAEs (7 and 1 due to COVID-19 infection). 26 deaths were due to non-TEAEs (defined as not considered related to study treatment and occurring either >112 days after cilta-cel or during subsequent therapy (cilta-cel [n=15]; standard-of-care [n=11]) (Table 11).

Of 176 patients receiving cilta-cel as study treatment, 134 (76.1%) experienced CRS (grade 1/2 [n=132]; grade 3 [n=2]). Median time to onset was 8 days (range, 1-23) and duration was 3 days (range, 1-17) (Table 12). 36 (20.5%) patients experienced CAR-T-related neurotoxicities (grade 1/2 [n=31]; grade 3/4 [n=5]). All ICANS (n=8; 4.5%) cases were grade 1/2 with median 9.5 days to onset (range, 6-15) and median 2 days duration (range, 1-6) (Table 13). One movement and neurocognitive TEAE case (grade 1) was reported in a male patient, who was refractory to bridging therapy and had previous grade 2 CRS (Table 14). Onset was on day 85 post infusion and was ongoing at data cut-off. Cranial nerve palsies, most commonly affecting cranial nerve VII, were reported in 16 (9.1%) patients (grade 1/2 [n=14]; grade 3 [n=2]), and median onset was 21 days post infusion (range, 17-60). 14 patients recovered by data cutoff. Five (2.8%) patients experienced CAR-T-related peripheral neuropathies with 2.3% grade 1/2, 0.6% grade 3 (Table 14).

Prior to clinical cut-off, there were 7 COVID-19-related deaths in the cilta-cel arm, of which 6 infections were diagnosed within 4 months of cilta-cel infusion, when patients were most immunocompromised. Further, this coincided with the emergence of the COVID-19 omicron variant and the relaxing of COVID-related restrictions in some regions. These deaths contributed to the higher number of fatal events observed in the cilta-cel vs. standard-of-care arm in the first year after randomization and highlight the need for strict prevention measures and aggressive treatment of COVID-19 in patients receiving CAR-T therapies. No COVID-19-related deaths occurred in the cilta-cel arm after safety measures consistent with international guidelines were introduced. Overall COVID-19 infection incidences were similar between cilta-cel and standard-of-care (33% vs. 27%). Furthermore, other treatment-emergent infections were comparable between cilta-cel and standard-of-care (62% vs. 71%), demonstrating that with appropriate prophylaxis and treatment, infection risk is generally manageable in patients receiving cilta-cel.

In the cilta-cel arm, there were 21 deaths in the first year after start of study treatment. Three deaths occurred in patients who did not receive cilta-cel (1 death due to an AE, 2 deaths due to AEs after start of subsequent therapy). Twelve deaths occurred due to AEs (including 6 due to COVID-19 pneumonia). An additional six deaths were due to AEs which occurred in patients who progressed during bridging therapy and received cilta-cel as subsequent therapy. In the second year, four deaths occurred due to AEs (including 1 due to COVID-19 pneumonia).

In the standard care arm, there were 11 deaths due to AEs in the first year after start of study treatment, including 5 deaths on study treatment (1 due to COVID-19 pneumonia) and 6 deaths after start of subsequent therapy. In the second year, there were 5 deaths due to AEs after start of subsequent therapy.

Overall, CAR-T-specific AEs were manageable with appropriate supportive care. Lower rates of cytopenias, CRS, and CAR-T-related neurotoxicity were seen in CARTITUDE-4 than in CARTITUDE-1, suggesting cilta-cel may be better tolerated when used earlier in treatment (Fonseca, et al., BMC Cancer 2020; 20:1087; Berdeja et al., Lancet 2021; 398:314-24). Movement and neurocognitive TEAE rates were also lower in CARTITUDE-4 (0.6%) than in CARTITUDE-1 (6%) (Fonseca, et al., BMC Cancer 2020; 20:1087; Berdeja et al., Lancet 2021; 398:314-24), likely related to patient management strategies implemented to mitigate this risk (Cohen et al., Blood Cancer J 2022; 12:32). Cranial nerve palsies observed in the study were mild to moderate; most cases had resolved at data cut-off.

CARTITUDE-4 demonstrated a favorable benefit-risk profile for a single infusion of cilta-cel compared with standard-of-care, with the results suggesting increased efficacy and improved tolerability when used earlier in treatment. The strong PFS benefit and rapid and deep response with cilta-cel, combined with the known high rates of attrition across LOT (de Arriba de la Fuente et al., Cancers (Basel) 2022;15), highlight the potential for cilta-cel to become a key therapy for patients with MM after first relapse.

TABLE 1
Baseline demographics and disease characteristics

| | Cilta-cel | Standard-of-care |
|---|---|---|
| Characteristic | (n = 208) | (n = 211) |
| Median age, years (range) | 61.5 (27-78) | 61.0 (35-80) |
| Male, n (%) | 116 (55.8) | 124 (58.8) |
| Race, n (%) | | |
| American Indian or Alaska Native | 1 (0.5) | 1 (0.5) |
| Asian | 16 (7.7) | 20 (9.5) |
| Black^{a} | 6 (2.9) | 7 (3.3) |
| White | 157 (75.5) | 157 (74.4) |
| Not reported | 28 (13.5) | 26 (12.3) |
| Ethnicity, n (%) | | |
| Hispanic or Latino | 18 (8.7) | 10 (4.7) |
| Not Hispanic or Latino | 152 (73.1) | 165 (78.2) |
| Not Reported | 38 (18.3) | 36 (17.1) |
| Geographic region | | |
| Europe | 128 (61.5) | 129 (61.1) |
| North America | 32 (15.4) | 32 (15.2) |
| Asia | 27 (13.0) | 25 (11.8) |
| Australia | 21 (10.1) | 25 (11.8) |
| ECOG Performance Status, n (%) | | |
| 0 | 114 (54.8) | 121 (57.3) |
| 1 | 93 (44.7) | 89 (42.2) |
| 2^{b} | 1 (0.5) | 1 (0.5) |
| ISS stage, n (%) | | |
| I | 136 (65.4) | 132 (62.6) |
| II | 60 (28.8) | 65 (30.8) |
| III | 12 (5.8) | 14 (6.6) |
| Median time since diagnosis, years | 3.0 (0.3-18.1) | 3.4 (0.4-22.1) |
| (range) | | |
| Presence of soft-tissue | 44 (21.2) | 35 (16.6) |
| plasmacytomas^{c}, n (%) | | |
| Bone marrow plasma cells ≥60%^{d}, | 42 (20.4) | 43 (20.7) |
| n (%) | | |
| Cytogenetic risk, n (%)^{e} | | |
| Standard risk | 69 (33.3) | 70 (33.3) |
| High-risk | 123 (59.4) | 132 (62.9) |
| Gain/amp(1q) | 89 (43.0) | 107 (51.0) |
| del(17p) | 49 (23.7) | 43 (20.5) |
| t(4; 14) | 30 (14.5) | 30 (14.3) |
| t(14; 16) | 3 (1.4) | 7 (3.3) |
| With ≥2 high-risk abnormalities | 43 (20.7) | 49 (23.2) |
| With del(17p), t(4:14), or | 73 (35.1) | 69 (32.7) |
| t(14; 16) | | |
| Unknown | 15 (7.2) | 8 (3.8) |
| Tumor BCMA expression ≥50%, | 141 (67.8) | 138 (65.4) |
| n (%) | | |
| Prior lines of therapy, n (%) | | |
| 1 | 68 (32.7) | 68 (32.2) |
| 2 | 83 (39.9) | 87 (41.2) |
| 3 | 57 (27.4) | 56 (26.5) |
| Prior immunomodulatory drugs, | 208 (100.0) | 211 (100.0) |
| n (%) | | |
| Lenalidomide | 208 (100.0) | 211 (100.0) |
| Pomalidomide | 8 (3.8) | 10 (4.7) |
| Prior anti-CD38 antibody | 53 (25.5) | 55 (26.1) |
| Daratumumab | 51 (24.5) | 54 (25.6) |
| Isatuximab | 2 (1.0) | 2 (0.9) |
| Prior proteasome inhibitors, | 208 (100.0) | 211 (100.0) |
| n (%) | | |
| Bortezomib | 203 (97.6) | 205 (97.2) |
| Carfilzomib | 77 (37.0) | 66 (31.3) |
| Ixazomib | 21 (10.1) | 21 (10.0) |
| Triple class^{f} exposed | 53 (25.5) | 55 (26.1) |
| Penta drug^{g} exposed | 14 (6.7) | 10 (4.7) |
| Refractory status, n (%) | | |
| Lenalidomide | 208 (100.0) | 211 (100.0) |
| Bortezomib | 55 (26.4) | 48 (22.7) |
| Carfilzomib | 51 (24.5) | 45 (21.3) |
| Any anti-CD38 antibody | 50 (24.0) | 46 (21.8) |
| Daratumumab | 48 (23.1) | 45 (21.3) |
| Ixazomib | 15 (7.2) | 17 (8.1) |
| Pomalidomide | 8 (3.8) | 9 (4.3) |
| Triple class^{f} | 30 (14.4) | 33 (15.6) |
| Penta drug^{g} | 2 (1.0) | 1 (0.5) |
| Any proteasome inhibitor | 103 (49.5) | 96 (45.5) |

^{a} Among patients enrolled in the United States, 9 (14.1%) were Black.
^{b}Latest, non-missing ECOG score on or prior to Apheresis/Cycle 1 Day 1 is used. All patients met the inclusion criteria of ECOG performance status of 0 or 1 prior to randomization.
^{c}Including extramedullary and bone-based plasmacytomas with measurable soft-tissue component.
^{d}in 206 (cilta-cel arm) and 208 (standard-of-care arm) patients; maximum value from bone marrow biopsy and bone marrow aspirate is selected if both the results are available.
^{e}In 207 (cilta-cel arm) and 210 (standard-of-care arm) patients.
^{f}Including 1 proteasome inhibitor, 1 immunomodulatory drug, and 1 anti-CD38 monoclonal antibody.
^{g}Including ≥2 proteasome inhibitors, ≥2 immunomodulatory drugs, and 1 anti-CD38 monoclonal antibody.
BCMA, B-cell maturation antigen; ECOG, Eastern Cooperative Oncology Group; ISS, International Staging System.

TABLE 2
Representativeness of study participants

| Category | Description |
|---|---|
| Disease, problem, or | Lenalidomide-refractory multiple myeloma |
| condition under | after one to three prior lines of therapy |
| investigation | |
| Special considerations | |
| related to | |
| Sex and gender | Multiple myeloma affects men more than |
| | women (3:2 ratio)^{1, 2} |
| Age | Multiple myeloma prevalence increases with |
| | age. The median age of patients at diagnosis |
| | is approximately 66-70 years of age, with |
| | increasing diagnoses in patients ≥80 years^{2, 3} |
| Race or ethnic group | Black patients have a higher incidence of |
| | multiple myeloma compared to other |
| | ethnicities (>2-fold higher than white |
| | patients), representing approximately 20% of |
| | the patients with multiple myeloma in the US⁴ |
| Geography | Multiple myeloma incidence and mortality |
| | appears highest in Western Europe, the |
| | United States, Canada and Australia. In |
| | recent decades, East Asia has seen the |
| | highest increase in incidence⁵ |
| Other considerations | |
| Overall | The patients enrolled in the present study |
| representativeness | had the expected ratio of men to women. |
| of this trial | The median age at study entry was 61 years |
| | (range, 27-80), which aligns well with the |
| | median age at diagnosis reported in the |
| | literature. |
| | Although the proportion of Black patients |
| | enrolled in the study overall (3.1%) was |
| | lower than their representation in the US |
| | population, 14.1% of patients enrolled in the |
| | US were Black. |

TABLE 3
Criteria for Response to Multiple Myeloma Treatment

| Response | Response Criteria |
|---|---|
| Stringent complete | CR as defined below, plus |
| response (sCR) | Normal FLC ratio, and |
| | Absence of clonal plasma cells (PCs) by immunohistochemistry or |
| | 2- to 4-color flow cytometry |
| Complete response | Negative immunofixation of serum and urine, and |
| (CR)^{a} | Disappearance of any soft tissue plasmacytomas, and |
| | <5% PCs in bone marrow |
| | No evidence of initial monoclonal protein isotype(s) on |
| | immunofixation of the serum and urine.^{b} |
| Very good partial | Serum and urine M-component detectable by immunofixation but |
| response (VGPR)^{a} | not on electrophoresis, or |
| | ≥90% reduction in serum M-component plus urine M-component <100 |
| | mg/24 hours |
| Partial response (PR) | ≥50% reduction of serum M-protein and reduction in 24-hour |
| | urinary M-protein by >90% or to <200 mg/24 hours |
| | If serum and urine M-protein were not measurable, a decrease ≥50% |
| | in the difference between involved and uninvolved FLC levels was |
| | required in place of the M-protein criteria |
| | If serum and urine M-protein were not measurable, and serum FLC |
| | assay was also not measurable, ≥50% reduction in bone marrow PCs |
| | was required in place of M-protein, provided baseline percentage had |
| | been ≥30% |
| | In addition to the above criteria, if present at baseline, ≥50% |
| | reduction in the size of soft tissue plasmacytomas was also required. |
| Minimal response (MR) | ≥25% but ≤49% reduction of serum M-protein and reduction in 24- |
| | hour urine M-protein by 50% to 89% |
| | In addition to the above criteria, if present at baseline, ≥50% |
| | reduction in the size of soft tissue plasmacytomas was also required. |
| Stable disease | Not meeting criteria for sCR, CR, VGPR, PR, MR, or progressive |
| | disease |
| Progressive disease^{c} | Any one or more of the following criteria: |
| | Increase of 25% from lowest response value in any of the following: |
| | Serum M-component (absolute increase must be ≥0.5 g/dL), |
| | and/or |
| | Urine M-component (absolute increase must be ≥200 mg/24 |
| | hours), and/or |
| | Only in subjects without measurable serum and urine M-protein |
| | levels: the difference between involved and uninvolved FLC |
| | levels (absolute increase must be >10 mg/dL) |
| | Only in subjects without measurable serum and urine M-protein |
| | levels and without measurable disease by FLC levels, bone |
| | marrow PC percentage (absolute increase must be ≥10%). |
| | Appearance of a new lesion(s), ≥50% increase from nadir in sum of |
| | the products of the maximal perpendicular diameters of measured |
| | lesions of >1 lesion, or ≥50% increase in the longest diameter of a |
| | previous lesion >1 cm in short axis |
| | Definite development of new bone lesions or definite increase in the |
| | size of existing bone lesions |
| | ≥50% increase in circulating plasma cells (minimum of 200 cells per |
| | μL) if this was the only measure of disease |

^{a}Clarifications to the criteria for coding CR and VGPR in subjects in whom the only measurable disease is by serum FLC levels: CR in such subjects indicates a normal FLC ratio of 0.26 to 1.65 in addition to CR criteria listed above. VGPR in such subjects requires a ≥90% decrease in the difference between involved and uninvolved FLC levels. For patients achieving very good partial response by other criteria, a soft tissue plasmacytoma must decrease by more than 90% in the sum of the maximal perpendicular diameter (SPD) compared with baseline.
^{b}In some cases it is possible that the original M protein light-chain isotype is still detected on immunofixation but the accompanying heavy-chain component has disappeared; this would not be considered as a CR even though the heavy-chain component is not detectable, since it is possible that the clone evolved to one that secreted only light chains. Thus, if a patient has IgA lambda myeloma, then to qualify as CR there should be no IgA detectable on serum or urine immunofixation; if free lambda is detected without IgA, then it must be accompanied by a different heavy chain isotype (IgG, IgM, etc.).
^{c}Clarifications to the criteria for coding progressive disease: bone marrow criteria for progressive disease are to be used only in subjects without measurable disease by M-protein and by FLC levels; “25% increase” refers to M-protein, and FLC, and does not refer to bone lesions, or soft tissue plasmacytomas and the “lowest response value” does not need to be a confirmed value.
Notes:
All response categories (CR, sCR, VGPR, PR, MR, and progressive disease) require 2 consecutive assessments made at any time before the institution of any new therapy; CR, sCR, VGPR, PR, MR, and stable disease categories also require no known evidence of progressive or new bone lesions if radiographic studies were performed. VGPR and CR categories require serum and urine studies regardless of whether disease at baseline was measurable on serum, urine, both, or neither.
Radiographic studies are not required to satisfy these response requirements. Bone marrow assessments need not be confirmed. For progressive disease, serum M-component increases of ≥1 g/dL are sufficient to define relapse if lowest M-component is ≥5 g/dL.

TABLE 4
Unweighted PFS (sensitivity analysis)

| | Cilta-cel | Standard-of-care |
|---|---|---|
| | (N = 208) | (N = 211) |
| Median (95% CI) | NE (22.83, NE) | 11.79 (9.66, 13.77) |

| | |
|---|---|
| Hazard ratio (95% CI) | 0.4 (0.29, 0.55) |
| P value | <0.0001 |

NE, not estimable.

TABLE 5
Treatment Responses and Minimal Residual Disease Negativity Rates in the Intent-to-Treat Population.

| | Cilta-cel | Standard-of- | Odds Ratio |
|---|---|---|---|
| | (n = 208) | care (n = 211) | (95% CI)^{a} |
| Overall response rate,^{b} n (%) | 176 (84.6) | 142 (67.3) | 3.0 (1.8-5.0) |
| Stringent complete response | 121 (58.2) | 32 (15.2) | |
| Complete response | 31 (14.9) | 14 (6.6) | |
| Very good partial response | 17 (8.2) | 50 (23.7) | |
| Partial response | 7 (3.4) | 46 (21.8) | |
| Minimal response | 1 (0.5) | 11 (5.2) | |
| Stable disease | 13 (6.3) | 47 (22.3) | |
| Progressive disease | 17 (8.2) | 6 (2.8) | |
| Not evaluable | 1 (0.5) | 5 (2.4) | |
| Complete response or better | 152 (73.1) | 46 (21.8) | 10.3 (6.5-16.4) |
| Very good partial response or better | 169 (81.3) | 96 (45.5) | 5.9 (3.7-9.4) |
| 12-month duration of response, % (95% CI) | 84.7 (78.1-89.4) | 63.0 (54.2-70.6) | |
| Duration of response, months median (95% CI) | NR | 16.6 (12.9-NE) | |
| Time to first response, median (range), months | 2.1 (0.9-11.1) | 1.2 (0.6-10.7) | |
| Time to best response, median (range), months | 6.4 (1.1-18.6) | 3.1 (0.8-20.6) | |
| Minimal residual disease negative,^{c, d} n (%) | 126 (60.6) | 33 (15.6) | 8.7 (5.4-13.9) |
| 12-month progression-free survival, % (95% CI) | 75.9 (69.4-81.1) | 48.6 (41.5-55.3) | |

^{a}Mantel-Haenszel estimate of the common odds ratio for stratified tables is used. An odds ratio >1 indicates an advantage for cilta-cel.
^{b}Includes patients who achieved partial response or better.
^{c}At 10⁻⁵ threshold, assessed by next-generation sequencing.
^{d}For minimal residual disease-evaluable patients: cilta-cel 88% (n = 144), standard-of-care 33% (n = 101).
NE, not estimable.
NR, not reached.

TABLE 6
Cytokine Release Syndrome ASTCT Consensus Grading System

| Grade | Toxicity |
|---|---|
| Grade 1 | Fever^{a} (Temperature ≥38°) |
| Grade 2 | Fever^{a} (Temperature ≥38°) with either: |
| | Hypotension not requiring vasopressors |
| | And/or^{c} hypoxia requiring low-flow nasal cannula^{b} or blow-by. |
| Grade 3 | Fever^{a} (Temperature ≥38°) with either: |
| | Hypotension requiring a vasopressor with or without |
| | vasopressin, |
| | And/or^{c} hypoxia requiring high-flow nasal cannula^{b}, facemask, |
| | nonrebreather mask, or Venturi mask. |
| Grade 4 | Fever^{a} (Temperature ≥38°) with either: |
| | hypotension requiring multiple vasopressors (excluding |
| | vasopressin), |
| | And/or^{c} hypoxia requiring positive pressure (eg, CPAP, BiPAP, |
| | intubation and mechanical ventilation). |
| Grade 5 | Death |

^{a}Fever not attributable to any other cause. In patients who have CRS then receive antipyretics or anticytokine therapy such as tocilizumab or steroids, fever is no longer required to grade subsequent CRS severity. In this case, CRS grading is driven by hypotension and/or hypoxia.
^{b}Low-flow nasal cannula is defined as oxygen delivered at ≤6 L/minute or blow-by oxygen delivery. High-flow nasal cannula is defined as oxygen delivered at >6 L/minute.
^{c}CRS grade is determined by the more severe event: hypotension or hypoxia not attributable to any other cause.
Note:
Organ toxicities associated with CRS may be graded according to CTCAE v5.0 but they do not influence CRS grading.

TABLE 7
Immune Effector Cell-associated Neurotoxicity Syndrome
(ICANS) ASTCT Consensus Grading Systema,b

| Neurotoxicity | | | | |
|---|---|---|---|---|
| Domain | Grade 1 | Grade 2 | Grade 3 | Grade 4 |
| ICE Score | 7-9 | 3-6 | 0-2 | 0 (patient is unarousable |
| | | | | and unable to perform ICE). |
| Depressed | Awakens | Awakens to | Awakens only to | Patient is unarousable or |
| Level of | spontaneously. | voice. | tactile stimulus. | requires vigorous or |
| Consciousness | | | | repetitive tactile stimuli |
| | | | | to arouse. Stupor or coma. |
| Seizure | N/A | N/A | Any clinical seizure, | Life-threatening |
| | | | focal or generalized, | prolonged seizure (>5 |
| | | | that resolves rapidly; | min); or Repetitive |
| | | | or Non-convulsive | clinical or electrical |
| | | | seizures on EEG that | seizures without return to |
| | | | resolve with intervention. | baseline in between. |
| Motor | N/A | N/A | N/A | Deep focal motor weakness such as |
| Findings | | | | hemiparesis or paraparesis. |
| Raised | N/A | N/A | Focal/local edema on | Diffuse cerebral edema on |
| Intracranial | | | neuroimaging. | neuroimaging; or Decerebrate or |
| Pressure/ | | | | decorticate posturing; or |
| Cerebral | | | | Cranial nerve VI palsy; or |
| Edema | | | | Papilledema; or Cushing's triad. |

a: Toxicity grading according to Lee et al 2019
bICANS grade is determined by the most severe event (ICE score, level of consciousness, seizure, motor findings, raised ICP/cerebral edema) not attributable to any other cause.
Note:
all other neurological adverse events (not associated with ICANS) should continue to be graded with CTCAE Version 5.0 during both phases of the study

TABLE 8
Treatment-emergent adverse events in the safety population

| | Cilta-cel | | Standard-of-care | |
|---|---|---|---|---|
| | (n = 208) | | (n = 208) | |
| | All | Grade 3/4 | All | Grade 3/4 |
| Any adverse event occurring in ≥15% | 208 (100) | 201 (96.6) | 208 (100) | 196 (94.2) |
| of patients in any arm, n (%) | | | | |
| Serious adverse event | 92 (44.2) | 67 (32.2) | 81 (38.9) | 70 (33.7) |
| Hematologic | 197 (94.7) | 196 (94.2) | 185 (88.9) | 179 (86.1) |
| Neutropenia | 187 (89.9) | 187 (89.9) | 177 (85.1) | 172 (82.2) |
| Thrombocytopenia | 113 (54.3) | 86 (41.3) | 65 (31.3) | 39 (18.8) |
| Anemia | 113 (54.3) | 74 (35.6) | 54 (26.0) | 30 (14.4) |
| Lymphopenia | 46 (22.1) | 43 (20.7) | 29 (13.9) | 25 (12.0) |
| Nonhematologic | | | | |
| Infections | 129 (62.0) | 56 (26.9) | 148 (71.2) | 51 (24.5) |
| Upper respiratory tract infections^{a} | 39 (18.8) | 4 (1.9) | 54 (26.0) | 4 (1.9) |
| COVID-19^{b} | 29 (13.9) | 6 (2.9) | 55 (26.4) | 12 (5.8) |
| Lower respiratory tract/lung infections^{c} | 19 (9.1) | 9 (4.3) | 36 (17.3) | 8 (3.8) |
| Other nonhematologic | | | | |
| Nausea | 101 (48.6) | 0 | 38 (18.3) | 2 (1.0) |
| Hypogammaglobulinemia | 88 (42.3) | 15 (7.2) | 13 (6.3) | 1 (0.5) |
| Diarrhea | 70 (33.7) | 8 (3.8) | 56 (26.9) | 5 (2.4) |
| Fatigue | 60 (28.8) | 4 (1.9) | 68 (32.7) | 2 (1.0) |
| Headache | 55 (26.4) | 0 | 27 (13.0) | 0 |
| Constipation | 49 (23.6) | 1 (0.5) | 44 (21.2) | 2 (1.0) |
| Hypokalaemia | 39 (18.8) | 8 (3.8) | 14 (6.7) | 3 (1.4) |
| Asthenia | 36 (17.3) | 1 (0.5) | 34 (16.3) | 5 (2.4) |
| Peripheral edema | 35 (16.8) | 0 | 24 (11.5) | 2 (1.0) |
| Decreased appetite | 34 (16.3) | 2 (1.0) | 11 (5.3) | 0 |
| Peripheral sensory neuropathy | 33 (15.9) | 0 | 38 (18.3) | 1 (0.5) |
| Back pain | 33 (15.9) | 2 (1.0) | 39 (18.8) | 2 (1.0) |
| Arthralgia | 32 (15.4) | 2 (1.0) | 25 (12.0) | 1 (0.5) |
| Pyrexia | 32 (15.4) | 0 | 32 (15.4) | 2 (1.0) |
| Dyspnea | 28 (13.5) | 1 (0.5) | 41 (19.7) | 1 (0.5) |
| Insomnia | 23 (11.1) | 2 (1.0) | 52 (25.0) | 6 (2.9) |
| CAR-T associated adverse events^{d} | (n = 176) | | | |
| Cytokine release syndrome (CRS) | 134 (76.1) | 2 (1.1) | | |
| Neurotoxicity^{e} | 36 (20.5) | 5 (2.8) | | |
| Immune effector cell-associated neurotoxicity | 8 (4.5) | 1^{f} | | |
| syndrome and associated symptoms (ICANS) | | | | |
| Other^{f} | 30 (17.0) | 4 (2.3) | | |
| Movement and neurocognitive treatment- | 1 (0.57) | 0 | | |
| emergent adverse event (MNTs) | | | | |

^{a}Includes preferred terms upper respiratory tract infection, nasopharyngitis, sinusitis, rhinitis, tonsillitis, pharyngitis, laryngitis, and pharyngotonsillitis.
^{b}Includes preferred terms COVID-19, COVID-19 pneumonia, and asymptomatic COVID-19. In addition to 6 (cilta-cel) and 12 (standard-of-care) grade 3/4 events, there were 7 and 1 grade 5 events, respectively.
^{c}Includes preferred terms pneumonia, bronchitis and lower respiratory tract infection.
^{d}Analyzed in the patients who received cilta-cel as study treatment (n = 176).
^{e}There were no fatal neurotoxicities.
^{f}Grade 3 syncope reported as a symptom of grade 2 immune effector cell-associated neurotoxicity syndrome.
^{g}Other neurotoxicities: includes adverse events reported as CAR-T cell neurotoxicity that are not ICANS or associated symptoms.

TABLE 9
Incidence of grade 3/4 cytopenias and prolonged grade
3/4 cytopenias occurring after cilta-cel infusion

| | Patients who received cilta-cel as study treatment (n = 176) | | |
|---|---|---|---|
| | Grade 3/4 | Prolonged (>30 days) | Prolonged (>60 days) |
| | events, n (%) | grade 3/4 events^{a}, n (%) | grade 3/4 events^{a}, n (%) |
| Lymphopenia | 176 (100) | 51 (29.0) | 18 (10.2) |
| Neutropenia | 167 (94.9) | 46 (26.1) | 18 (10.2) |
| Thrombocytopenia | 72 (40.9) | 46 (26.1) | 19 (10.8) |
| Anemia | 52 (29.5) | 3 (1.7) | 2 (1.1) |

^{a}Defined as initial grade 3/4 events that did not recover to grade ≤2 by day 30 or 60, per laboratory results.

TABLE 10
Second primary malignancies after treatment with
cilta-cel or standard-of-care (safety population)

| | Cilta-cel | Standard-of-care |
|---|---|---|
| | (n = 208) | (n = 208) |
| Patients with second primary | 9 (4.3) | 14 (6.7) |
| malignancies | | |
| Cutaneous/non-invasive malignancies | 5 (2.4) | 10 (4.8) |
| Basal cell carcinoma | 2 (1.0) | 7 (3.4) |
| Bowen's disease | 0 | 2 (1.0) |
| Lip squamous cell carcinoma | 0 | 1 (0.5) |
| Malignant melanoma | 1 (0.5) | 0 |
| Malignant melanoma in situ | 1 (0.5) | 0 |
| Squamous cell carcinoma of skin | 2 (1.0) | 4 (1.9) |
| Hematologic malignancies | 3 (1.4) | 0 |
| Acute myeloid leukemia | 1 (0.5) | 0 |
| Myelodysplastic syndrome^{a} | 1 (0.5) | 0 |
| Peripheral T-cell lymphoma | 1 (0.5) | 0 |
| Non-cutaneous/invasive malignancies | 1 (0.5) | 4 (1.9) |
| Angiosarcoma | 1 (0.5) | 0 |
| Invasive lobular breast carcinoma | 0 | 1 (0.5) |
| Pleomorphic malignant fibrous | 0 | 1 (0.5) |
| histiocytoma | | |
| Renal cell carcinoma | 0 | 1 (0.5) |
| Tonsil cancer | 0 | 1 (0.5) |

^{a}At study entry, patient had essential thrombocythemia.

TABLE 11
Causes of death (safety population)

| | Cilta-cel | Standard-of-care |
|---|---|---|
| | (n = 208) | (n = 208) |
| Deaths, n (%) | 39 (18.8) | 46 (22.1) |
| Progressive disease | 14 (6.7) | 30 (14.4) |
| Non-treatment emergent adverse event^{a} | 15 (7.2) | 11 (5.3) |
| Treatment-emergent adverse event | 10 (4.8)^{b} | 5 (2.4) |
| COVID-19 pneumonia^{b} | 7 (3.4) | 1 (0.5) |
| Neutropenic sepsis | 1 (0.5) | 0 |
| Pneumonia | 1 (0.5) | 0 |
| Progressive multifocal | 0 | 1 (0.5) |
| leukoencephalopathy | | |
| Respiratory tract infection | 0 | 1 (0.5) |
| Septic shock | 0 | 1 (0.5) |
| Respiratory failure | 1 (0.5)^{c} | 0 |
| Pulmonary embolism | 0 | 1 (0.5) |

^{a}Adverse events were considered non treatment-emergent if they were not considered related to study treatment, and they occurred either >112 days after cilta-cel or after the start of subsequent therapy; for the standard care arm, adverse events were considered non-treatment emergent if they were not considered related to study treatment (DPd or PVd), and they occurred more than 30 days after the last dose of study treatment or after the start of subsequent therapy, whichever came first. . .
^{b}4 cilta-cel patients received 2 or 3 COVID-19 vaccinations prior to receiving cilta-cel, and 3 patients received no vaccines prior to cilta-cel. 2 of 7 patients received COVID-19 vaccination after cilta-cel, 1 dose each. All patients had a response of PR or better to study treatment and did not progress prior to COVID-19 infection.
^{c}Occurred before infusion of cilta-cel.

TABLE 12
Characteristics and management of cytokine release syndrome
patients receiving cilta-cel as study treatment

| | Patients who received cilta-cel |
|---|---|
| | as study treatment (n = 176) |
| Patients with a cytokine release | 134 (76.1) |
| syndrome event, n (%) | |
| Maximum toxicity grade, n (%) | |
| Grade 1 | 93 (52.8) |
| Grade 2 | 39 (22.2) |
| Grade 3 | 2 (1.1) |
| Grade 4 | 0 |
| Grade 5 | 0 |
| Median time to first onset, | 8.0 (1-23) |
| days (range) | |
| Median duration, days (range) | 3.0 (1-17) |
| Resolved | 134 |
| Supportive treatments, n | 131 |
| Tocilizumab | 71 |
| Oxygen | 17 |
| Corticosteroids | 8 |
| Vasopressor | 2 |

TABLE 13
Characteristics and management of immune
effector-cell-associated neurotoxicities

| | Patients who received cilta-cel |
|---|---|
| | as study treatment (n = 176) |
| Patients with immune effector-cell- | 8 (4.5) |
| associated neurotoxicities, n (%) | |
| Maximum toxicity grade, n (%) | |
| Grade 1 | 6 (3.4) |
| Grade 2 | 2 (1.1) |
| Grade 3 | 0 |
| Grade 4 | 0 |
| Grade 5 | 0 |
| Median time to onset, days (range) | 9.5 (6-15) |
| Median duration, days (range) | 2.0 (1-6) |
| Resolved | 8 |
| Supportive treatments, n | 4 |
| Corticosteroids | 4 |
| Tocilizumab | 2 |

TABLE 14
Other CAR-T cell neurotoxicities

| | Patients who received cilta-cel |
|---|---|
| | as study treatment (n = 176) |
| Other neurotoxicity,^{a} n (%) | 30 (17.0) |
| Grade 3/4^{b} | 4 (2.3) |
| Grade 5 | 0 |
| Cranial nerve palsy, n (%) | 16 (9.1) |
| Grade 2 | 14 (8.0) |
| Grade 3 | 2 (1.1) |
| Time from cilta-cel infusion to | 21 (17-60) |
| onset, median (range), days | |
| Cranial nerves affected, n | |
| III | 1 |
| V | 1 |
| VII | 16 |
| Duration, median (range), days | 77 (15-262) |
| Recovered/resolved, n | 14 |
| Supportive treatments, n | |
| Corticosteroids | 14 |
| Peripheral neuropathy, n (%) | 5 (2.8) |
| Grade 1 | 2 (1.1) |
| Grade 2 | 2 (1.1) |
| Grade 3 | 1 (0.6) |
| Time from cilta-cel infusion to | 63 (31-127) |
| onset, median (range), days | |
| Duration, median (range), days | 201 (107-503) |
| Recovered/resolved, n | 3 |
| Movement and neurocognitive | 1 (0.6) |
| treatment-emergent adverse | |
| events,^{c} n (%) | |
| Grade 1 | 1 (0.6) |
| Time from cilta-cel infusion to | 85 |
| onset, median (range), days | |
| Resolved | 0 |

^{a}Investigator-assessed non-ICANS neurotoxicity graded per National Cancer Institute Common Terminology Criteria for Adverse Events (NCI-CTCAE), version 5.0.
^{b}One case each of grade 3/4 neuralgia, third cranial nerve paralysis, polyneuropathy; and 1 patient with both trigeminal palsy and facial paralysis.
^{c}Onset day 85; included balance disorder, bradykinesia, extrapyramidal disorder, gait disturbance, micrographia, parkinsonism, psychomotor retardation, and reduced facial expression, all grade 1. Treated with levodopa and carbidopa monohydrate. Ongoing at data cut-off. This patient was male, was refractory to DPd bridging therapy, and had grade 2 cytokine release syndrome (risk factors for movement and neurocognitive treatment-emergent adverse events).

TABLE 15
Treatment responses and minimal residual disease negativity
rates in patients who received cilta-cel as study treatment

| | Cilta-cel as study |
|---|---|
| | treatment (n = 176) |
| Overall response rate,^{a} n (%) | 175 (99.4) |
| Stringent complete response | 121 (68.8) |
| Complete response | 31 (17.6) |
| Very good partial response | 17 (9.7) |
| Partial response | 6 (3.4) |
| Minimal response | 0 |
| Stable disease | 1 (0.6) |
| Progressive disease | 0 |
| Not evaluable | 0 |
| Complete response or better | 152 (86.4) |
| Very good partial response or better | 169 (96.0) |
| Median duration of response (95% CI), months | NE (NE-NE) |
| Time to first response, median (range), months | 2.1 (0.9-11.1) |
| Time to best response, median (range), months | 6.5 (1.1-18.6) |
| Minimal residual disease negative,^{b} n (%) | 126 (71.6) |
| 12-month progression-free survival, % (95% CI) | 89.7 (84.1-93.4) |

^{a}Includes patients who achieved partial response or better.
^{b}At 10⁻⁵ threshold, assessed by next-generation sequencing.
NE, not estimable.

TABLE 16
Serious adverse events in the safety population.

| | Cilta-cel | Standard care |
|---|---|---|
| | (n = 208) | (n = 208) |
| | All | All |
| Any serious adverse event, n (%) | 92 (44.2) | 81 (38.9) |
| Serious adverse events occurring | | |
| in ≥1% of patients in any arm, n (%) | | |
| Infections | 50 (24.0) | 51 (24.5) |
| COVID-19 pneumonia | 12 (5.8) | 9 (4.3) |
| Pneumonia | 6 (2.9) | 9 (4.3) |
| COVID-19 | 5 (2.4) | 4 (1.9) |
| Upper respiratory tract infection | 3 (1.4) | 4 (1.9) |
| Cytomegalovirus infection | 2 (1.0) | 1 (0.5) |
| Lower respiratory tract infection | 2 (1.0) | 0 |
| Respiratory tract infection | 2 (1.0) | 2 (1.0) |
| Sepsis | 2 (1.0) | 0 |
| Staphylococcal infection | 2 (1.0) | 0 |
| Cellulitis | 1 (0.5) | 2 (1.0) |
| Parainfluenzae virus infection | 1 (0.5) | 4 (1.9) |
| Pneumocystis jirovecii pneumonia | 1 (0.5) | 3 (1.4) |
| Urinary tract infection | 1 (0.5) | 2 (1.0) |
| Pneumonia legionella | 0 | 2 (1.0) |
| Rhinovirus infection | 0 | 3 (1.4) |
| Blood and lymphatic system disorders | 15 (7.2) | 9 (4.3) |
| Febrile neutropenia | 5 (2.4) | 5 (2.4) |
| Anemia | 4 (1.9) | 1 (0.5) |
| Neutropenia | 4 (1.9) | 1 (0.5) |
| Nervous system disorders | 14 (6.7) | 3 (1.4) |
| Facial paralysis | 9 (4.3) | 1 (0.5) |
| General disorders and administration | 8 (3.8) | 7 (3.4) |
| site conditions | | |
| Pyrexia | 4 (1.9) | 5 (2.4) |
| General physical health deterioration | 3 (1.4) | 0 |
| Immune system disorders | 7 (3.4) | 1 (0.5) |
| Cytokine release syndrome | 7 (3.4) | 1 (0.5) |
| Metabolism and nutrition disorders | 7 (3.4) | 3 (1.4) |
| Hypercalcemia | 5 (2.4) | 2 (1.0) |
| Gastrointestinal disorders | 6 (2.9) | 3 (1.4) |
| Diarrhea | 5 (2.4) | 0 |
| Cardiac disorders | 5 (2.4) | 4 (1.9) |
| Atrial fibrillation | 1 (0.5) | 2 (1.0) |
| Musculoskeletal and connective | 5 (2.4) | 3 (1.4) |
| tissue disorders | | |
| Back pain | 2 (1.0) | 0 |
| Respiratory, thoracic and mediastinal | 5 (2.4) | 7 (3.4) |
| disorders | | |
| Pleural effusion | 2 (1.0) | 0 |
| Respiratory failure | 2 (1.0) | 1 (0.5) |
| Pulmonary embolism | 1 (0.5) | 4 (1.9) |
| Neoplasms benign, malignant and | 4 (1.9) | 5 (2.4) |
| unspecified (incl cysts and polyps) | | |
| Injury, poisoning and procedural | 3 (1.4) | 2 (1.0) |
| complications | | |
| Psychiatric disorders | 2 (1.0) | 2 (1.0) |
| Renal and urinary disorders | 2 (1.0) | 3 (1.4) |
| Acute kidney injury | 2 (1.0) | 3 (1.4) |
| Investigations^{a} | 1 (0.5) | 2 (1.0) |
| Vascular disorders | 0 | 3 (1.4) |
| Deep vein thrombosis | 0 | 2 (1.0) |

The teachings of all patents, published applications, and references cited herein are incorporated by reference in their entirety.

While example aspects and embodiments have been particularly shown and described, it will be understood by those skilled in the art that various changes in form and details may be made therein without departing from the scope of the aspects and embodiments encompassed by the appended claims.

SEQUENCES

SEQ ID NO: 1-Ciltacabtagene autoleucel CAR CD8α signal peptide, CD8a SP amino
acid sequence
MALPVTALLLPLALLLHAARP
SEQ ID NO: 2-Ciltacabtagene autoleucel CAR BCMA binding domain, VHH1 amino
acid sequence
QVKLEESGGGLVQAGRSLRLSCAASEHTFSSHVMGWFRQAPGKERESVAVIGWRDI
STSYADSVKGRFTISRDNAKKTLYLQMNSLKPEDTAVYYCAARRIDAADFDSWGQG
TQVTVSS
SEQ ID NO: 3-Ciltacabtagene autoleucel CAR BCMA binding domain, G4S linker
amino acid sequence
GGGGS
SEQ ID NO: 4-Ciltacabtagene autoleucel CAR BCMA binding domain, VHH2 amino
acid sequence
EVQLVESGGGLVQAGGSLRLSCAASGRTFTMGWFRQAPGKEREFVAAISLSPTLAY
YAESVKGRFTISRDNAKNTVVLQMNSLKPEDTALYYCAADRKSVMSIRPDYWGQG
TQVTVSS
SEQ ID NO: 5-Ciltacabtagene autoleucel CAR CD8α hinge amino acid sequence
TTTPAPRPPTPAPTIASQPLSLRPEACRPAAGGAVHTRGLDFACD
SEQ ID NO: 6-Ciltacabtagene autoleucel CAR CD8α transmembrane amino acid
sequence
IYIWAPLAGTCGVLLLSLVITLYC
SEQ ID NO: 7-Ciltacabtagene autoleucel CAR CD137 Cytoplasmic amino acid
sequence
KRGRKKLLYIFKQPFMRPVQTTQEEDGCSCRFPEEEEGGCEL
SEQ ID NO: 8-Ciltacabtagene autoleucel CAR CD3ζ Cytoplasmic amino acid
sequence
RVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQAL
PPR
SEQ ID NO: 9-Ciltacabtagene autoleucel CAR CD8α signal peptide CD8α SP nucleic
acid sequence
ATGGCTCTGCCCGTCACCGCTCTGCTGCTGCCTCTGGCTCTGCTGCTGCACGCTG
CTCGCCCT
SEQ ID NO: 10-Ciltacabtagene autoleucel CAR BCMA binding domain, VHH1
nucleic acid sequence
CAGGTCAAACTGGAAGAATCTGGCGGAGGCCTGGTGCAGGCAGGACGGAGCCT
GCGCCTGAGCTGCGCAGCATCCGAGCACACCTTCAGCTCCCACGTGATGGGCTG
GTTTCGGCAGGCCCCAGGCAAGGAGAGAGAGAGCGTGGCCGTGATCGGCTGGA
GGGACATCTCCACATCTTACGCCGATTCCGTGAAGGGCCGGTTCACCATCAGCCG
GGACAACGCCAAGAAGACACTGTATCTGCAGATGAACAGCCTGAAGCCCGAGG
ACACCGCCGTGTACTATTGCGCAGCAAGGAGAATCGACGCAGCAGACTTTGATT
CCTGGGGCCAGGGCACCCAGGTGACAGTGTCTAGC
SEQ ID NO: 11-Ciltacabtagene autoleucel CAR BCMA binding domain, G4S linker
nucleic acid sequence
GGAGGAGGAGGATCT
SEQ ID NO: 12-Ciltacabtagene autoleucel CAR BCMA binding domain, VHH2
nucleic acid sequence
GAGGTGCAGCTGGTGGAGAGCGGAGGCGGCCTGGTGCAGGCCGGAGGCTCTCTG
AGGCTGAGCTGTGCAGCATCCGGAAGAACCTTCACAATGGGCTGGTTTAGGCAG
GCACCAGGAAAGGAGAGGGAGTTCGTGGCAGCAATCAGCCTGTCCCCTACCCTG
GCCTACTATGCCGAGAGCGTGAAGGGCAGGTTTACCATCTCCCGCGATAACGCC
AAGAATACAGTGGTGCTGCAGATGAACTCCCTGAAACCTGAGGACACAGCCCTG
TACTATTGTGCCGCCGATCGGAAGAGCGTGATGAGCATTAGACCAGACTATTGG
GGGCAGGGAACACAGGTGACCGTGAGCAGC
SEQ ID NO: 13-Ciltacabtagene autoleucel CAR CD8α hinge nucleic acid sequence
ACCACGACGCCAGCGCCGCGACCACCAACACCGGCGCCCACCATCGCGTCGCAG
CCCCTGTCCCTGCGCCCAGAGGCGTGCCGGCCAGCGGCGGGGGGCGCAGTGCAC
ACGAGGGGGCTGGACTTCGCCTGTGAT
SEQ ID NO: 14-Ciltacabtagene autoleucel CAR CD8α transmembrane nucleic acid
sequence
ATCTACATCTGGGCGCCCTTGGCCGGGACTTGTGGGGTCCTTCTCCTGTCACTGG
TTATCACCCTTTACTGC
SEQ ID NO: 15-Ciltacabtagene autoleucel CAR CD137 Cytoplasmic nucleic acid
sequence
AAACGGGGCAGAAAGAAACTCCTGTATATATTCAAACAACCATTTATGAGACCA
GTACAAACTACTCAAGAGGAAGATGGCTGTAGCTGCCGATTTCCAGAAGAAGAA
GAAGGAGGATGTGAACTG
SEQ ID NO: 16-Ciltacabtagene autoleucel CAR CD3z Cytoplasmic nucleic acid
sequence
AGAGTGAAGTTCAGCAGGAGCGCAGACGCCCCCGCGTACCAGCAGGGCCAGAA
CCAGCTCTATAACGAGCTCAATCTAGGACGAAGAGAGGAGTACGATGTTTTGGA
CAAGAGACGTGGCCGGGACCCTGAGATGGGGGGAAAGCCGAGAAGGAAGAACC
CTCAGGAAGGCCTGTACAATGAACTGCAGAAAGATAAGATGGCGGAGGCCTACA
GTGAGATTGGGATGAAAGGCGAGCGCCGGAGGGGCAAGGGGCACGATGGCCTT
TACCAGGGTCTCAGTACAGCCACCAAGGACACCTACGACGCCCTTCACATGCAG
GCCCTGCCCCCTCGCTAA
SEQ ID NO: 17-Ciltacabtagene autoleucel CAR amino acid sequence
MALPVTALLLPLALLLHAARPQVKLEESGGGLVQAGRSLRLSCAASEHTFSSHVMG
WFRQAPGKERESVAVIGWRDISTSYADSVKGRFTISRDNAKKTLYLQMNSLKPEDT
AVYYCAARRIDAADFDSWGQGTQVTVSSGGGGSEVQLVESGGGLVQAGGSLRLSC
AASGRTFTMGWFRQAPGKEREFVAAISLSPTLAYYAESVKGRFTISRDNAKNTVVLQ
MNSLKPEDTALYYCAADRKSVMSIRPDYWGQGTQVTVSSTSTTTPAPRPPTPAPTIA
SQPLSLRPEACRPAAGGAVHTRGLDFACDIYIWAPLAGTCGVLLLSLVITLYCKRGR
KKLLYIFKQPFMRPVQTTQEEDGCSCRFPEEEEGGCELRVKFSRSADAPAYQQGQNQ
LYNELNLGRREEYDVLDKRRGRDPEMGGKPRRKNPQEGLYNELQKDKMAEAYSEI
GMKGERRRGKGHDGLYQGLSTATKDTYDALHMQALPPR
SEQ ID NO: 18-Ciltacabtagene autoleucel CAR BCMA binding domain, VHH1 CDR1
SHVMG
SEQ ID NO: 19-Ciltacabtagene autoleucel CAR BCMA binding domain, VHH1 CDR2
VIGWRDISTSYADSVKG
SEQ ID NO: 20-Ciltacabtagene autoleucel CAR BCMA binding domain, VHH1 CDR3
ARRIDAADFDS
SEQ ID NO: 21-Ciltacabtagene autoleucel CAR BCMA binding domain, VHH2 CDR1
TFTMG
SEQ ID NO: 22-Ciltacabtagene autoleucel CAR BCMA binding domain, VHH2 CDR2
AISLSPTLAYYAESVKG
SEQ ID NO: 23-Ciltacabtagene autoleucel CAR BCMA binding domain, VHH2 CDR3
ADRKSVMSIRPDY

1. A method of treating a subject, comprising administering to the subject a dose of T cells comprising a chimeric antigen receptor (CAR) comprising:
(a) an extracellular antigen binding domain capable of specifically binding to an epitope of B-cell maturation antigen (BCMA),
(b) a transmembrane domain, and
(c) an intracellular signaling domain,
wherein the subject has multiple myeloma, has received one to three prior lines of therapies, including a therapy with an immunomodulatory drug (IMiD), and is refractory to the IMiD, wherein optionally the subject has a high-risk feature, further wherein optionally the high-risk feature is a cytogenetic abnormality, International Staging System (ISS) stage III, and/or soft tissue plasmacytomas, further wherein optionally the IMiD is lenalidomide.

2. (canceled)

3. A method of selectively treating a subject, comprising:
(1) determining whether the subject has a high-risk feature, the high-risk feature being a cytogenetic abnormality, International Staging System (ISS) stage III, and/or soft tissue plasmacytomas; and
(2) administering to the subject who is determined to have the high-risk feature in step (1) a dose of T cells comprising a chimeric antigen receptor (CAR) comprising:
(a) an extracellular antigen binding domain capable of specifically binding to an epitope of BCMA,
(b) a transmembrane domain, and
(c) an intracellular signaling domain,
wherein optionally the subject has multiple myeloma, has received one to three prior lines of therapies, including a therapy with an IMiD, and is refractory to the IMiD, further wherein optionally the IMiD is lenalidomide.

4. A method of selectively treating a subject, comprising administering to the subject who has been determined to have a high-risk feature a dose of T cells comprising a chimeric antigen receptor (CAR) comprising:
(a) an extracellular antigen binding domain capable of specifically binding to an epitope of BCMA,
(b) a transmembrane domain, and
(c) an intracellular signaling domain,
wherein the high-risk feature is a cytogenetic abnormality, International Staging System (ISS) stage III, and/or soft tissue plasmacytomas,
wherein optionally the subject has multiple myeloma, has received one to three prior lines of therapies, including a therapy with an IMiD, and is refractory to the IMiD, further wherein optionally the IMiD is lenalidomide.

5. (canceled)

6. The method of claim 1, wherein:
(a) the high-risk feature is a cytogenetic abnormality, wherein optionally:
(1) the cytogenetic abnormality is a high-risk cytogenetic abnormality, further wherein optionally the subject has one or more high-risk cytogenetic abnormality selected from a group comprising Gain/amp(1q), del(17p), t(4;14), t(14;16), or any combination thereof, further wherein optionally the subject has at least two cytogenetic abnormalities, and wherein optionally the subject has two, three, four, five, or more cytogenetic abnormalities; or
(2) the cytogenetic abnormality is a standard-risk cytogenetic abnormality;
(b) the high-risk feature is International Staging System (ISS) stage III; or
(c) soft tissue plasmacytomas.

7-16. (canceled)

17. The method of claim 1, wherein the subject has received one, two, or three, prior lines of therapy, wherein optionally the one, two or three prior lines of therapy comprises treatment with pomalidomide, wherein optionally the one, two or three prior lines of therapy further comprises:
(1) treatment with an anti-CD38 antibody, and wherein optionally the anti-CD38 antibody is daratumumab and/or isatuximab; or
(2) treatment with a proteasome inhibitor, wherein optionally the proteasome inhibitor is bortezomib, carfilzomib, ixazomib, or any combination thereof.

18-22. (canceled)

23. The method of claim 1, wherein the subject has further received:
(1) a bridging therapy, wherein optionally the bridging therapy is of the physician's choice, wherein optionally the bridging therapy comprises pomalidomide, bortezomib, dexamethasone, daratumumab, or any combination thereof, further wherein optionally the bridging therapy comprises pomalidomide, bortezomib and dexamethasone, and further wherein optionally the bridging therapy comprises daratumumab, pomalidomide and dexamethasone, further wherein optionally the subject has received the bridging therapy from about every 20 days to about every 30 days, further wherein optionally the subject has received the bridging therapy about every 21 days, further wherein optionally the subject has received the bridging therapy about every 28 days, and further wherein optionally the subject has received at least one, two, three, four, or more bridging therapies; or
(2) a lymphodepletion therapy, wherein optionally the lymphodepletion therapy comprises cyclophosphamide and/or fludarabine daily, wherein optionally the lymphodepletion therapy comprises cyclophosphamide and fludarabine daily, further wherein optionally the lymphodepletion therapy comprises cyclophosphamide at a concentration of about 300 mg/m2 and fludarabine at a concentration of about 30 mg/m2 daily for 3 days.

24-25. (canceled)

26. The method of claim 1, wherein the dose of the T cells is 0.5-1.0×10⁶ cells/kg of body weight of the subject, wherein optionally the dose of the T cells is about 0.75×10⁶ cells/kg of body weight of the subject, wherein optionally the method comprises administering the dose of the T cells about 5 to about 7 days after the start of the lymphodepletion therapy, wherein optionally, the dose is administered as a single infusion.

27. The method of claim 1, wherein the method is effective in obtaining an overall response in the subject after administering to the subject the dose of the T cells, wherein optionally the method is effective in obtaining the overall response at a rate of about 75% to about 100%, further wherein optionally the method is effective in obtaining the overall response at a rate of about 84.6%, further wherein optionally the method is effective in obtaining the overall response at a rate of about 99.4%, further wherein optionally the overall response comprises, in order from best to worst:
(1) a stringent complete response;
(2) a complete response;
(3) a very good partial response;
(4) a partial response; or
(5) a minimal response;
further wherein optionally:
(a) the overall response is a stringent complete response, wherein optionally the method is effective in obtaining the stringent complete response at a rate of about 40% to about 90%, about 50% to about 80%, about 58.2%, or about 68.8%;
(b) the overall response is a complete response, wherein optionally the method is effective in obtaining the complete response at a rate of about 10% to about 20%, further wherein optionally the method is effective in obtaining the complete response at a rate of about 14.9% or about 17.6%;
(c) the overall response is a very good partial response or a partial response;
(d) the method is effective in obtaining a stringent complete response or a complete response at a rate of about 70% to about 90%, wherein optionally the method is effective in obtaining a stringent complete response or a complete response at a rate of about 73.1% or about 86.4%;
(e) the method is effective in obtaining a stringent complete response, a complete response, or a very good partial response at a rate of about 80% to about 100%, wherein optionally the method is effective in obtaining a stringent complete response, a complete response, or a very good partial response at a rate of about 81.3% or about 96.0%;
(f) the method is effective in obtaining a minimal response;
(g) the method is effective in further obtaining minimal residual disease negative, wherein optionally the method is effective in obtaining minimal residual disease negative at a rate of about 50% to about 80%, further wherein optionally the method is effective in obtaining minimal residual disease negative at a rate of about 60.6% or about 71.6%;
(h) the method is effective in further obtaining 12-month progression-free survival for at least about 60 to about 100% of subjects, at least about 69.4 to about 81.1% of subjects, or at least about 84.1 to about 93.4% of subjects, wherein optionally the method is effective in obtaining 12-month progression-free survival for at least about 75.9% of subjects or about 89.7% of subjects;
(i) time to first overall response or first minimal response ranges about 0.9 to about 11.1 months, wherein optionally the time to the first overall response or the first minimal response is at a median of about 2.1 months; or
(j) time to best overall response or best minimal response is about 1.1 to about 18.6 months, wherein optionally the time to the best overall response or the best minimal response is at a median of about 6.4 or about 6.5 months.

28-33. (canceled)

34. The method of claim 1, wherein the method further comprises:
(1) treating the subject for an adverse event after administering the dose of the T cells, wherein optionally the method comprises administering a treatment to the subject to alleviate the adverse event, wherein optionally the adverse event comprises a hematologic adverse event, a nonhematologic adverse event, a treatment-emergent adverse event, or any combination thereof, wherein optionally the nonhematologic adverse event comprises an infection and/or a nonhematologic adverse event other than an infection, further wherein optionally the adverse event comprises neutropenia, thrombocytopenia, anemia, lymphopenia, an upper respiratory tract infection, nasopharyngitis, sinusitis, rhinitis, tonsillitis, pharyngitis, laryngitis, pharyngotonsillitis, COVID-19, COVID-19 pneumonia, asymptomatic COVID-19, neutropenic sepsis, progressive multifocal leukoencephalpathy, septic shock, respiratory failure, pulmonary embolism, a lower respiratory tract/lung infection, pneumonia, bronchitis, nausea, hypogammaglobulinemia, diarrhea, fatigue, headache, constipation, hypokalemia, asthenia, peripheral edema, decreased appetite, peripheral sensory neuropathy, back pain, arthralgia, pyrexia, dyspnea, insomnia, or any combination thereof, further wherein optionally the adverse event is a Grade 3/4 adverse event, further wherein optionally the adverse event lasts longer than about 30 days or about 60 days, further wherein optionally the adverse event occurs in the subject at a rate comparable to a rate of a same adverse event occurring in a subject undergoing a standard of care; or
(2) treating the subject for a second primary malignancy after administering the dose of the T cells, wherein optionally the method comprises administering a treatment to the subject to alleviate the second primary malignancy, wherein optionally the second primary malignancy comprises a cutaneous/non-invasive malignancy, a hematologic malignancy, a non-cutaneous/invasive malignancy, or any combination thereof, further wherein optionally the second primary malignancy comprises basal cell carcinoma, Bowen's disease, lip squamous cell carcinoma, malignant melanoma, malignant melanoma in situ, squamous cell carcinoma of skin, acute myeloid leukemia, a myelodysplastic syndrome, peripheral T-cell lymphoma, angiosarcoma, invasive lobular breast carcinoma, pleomorphic malignant fibrous histiocytoma, renal cell carcinoma, tonsil cancer, or any combination thereof, further wherein optionally the second primary malignancy occurs in the subject at a rate comparable to a rate of a same second primary malignancy occurring in a subject undergoing a standard of care.

35-36. (canceled)

37. The method of claim 1, wherein the method further comprises treating the subject for a CAR-T-associated adverse event after administering the dose of the T cells, wherein optionally the method comprises administering a treatment to the subject to alleviate the CAR-T-associated adverse event, wherein optionally the CAR-T-associated adverse event comprises a cytokine release syndrome (CRS) and/or neurotoxicity, wherein optionally:
(a) the CAR-T-associated adverse event is a CRS, further wherein optionally the CRS occurs in the subject at a rate of about 60% to about 90%, or a rate of about 76.1%, further wherein optionally maximum toxicity grade of the CRS is Grade 1, Grade 2 or Grade 3, further wherein optionally:
(1) the maximum toxicity grade of the CRS is Grade 1, wherein optionally the maximum toxicity grade of Grade 1 in the subject occurs at a rate of about 52.8%;
(2) the maximum toxicity grade of the CRS is Grade 2, wherein optionally the maximum toxicity grade of Grade 2 in the subject occurs at a rate of about 22.2%;
(3) the maximum toxicity grade of the CRS is Grade 3, wherein optionally the maximum toxicity grade of Grade 3 in the subject occurs at a rate of about 1.1%;
(4) time to first onset of the CRS ranges from about 1 to about 23 days, wherein optionally the time to the first onset of the CRS is at a median of about 8 days;
(5) duration of the CRS ranges from about 1 to about 17 days, wherein optionally the duration of the CRS is at a median of about 3 days; or
(6) the treatment comprises tocilizumab, oxygen, a corticosteroid, a vasopressor, or any combination thereof, or
(b) the CAR-T-associated adverse event is neurotoxicity, wherein optionally the neurotoxicity comprises an immune effector cell-associated neurotoxicity syndrome or associated symptom, movement and neurocognitive neurotoxicity, treatment-emergent adverse event of neurotoxicity, a non-immune effector cell-associated neurotoxicity syndrome or associated symptom, or any combination thereof, wherein optionally the neurotoxicity is an immune effector cell-associated neurotoxicity syndrome or associated symptom, further wherein optionally:
(1) the immune effector cell-associated neurotoxicity syndrome or associated symptom in the subject occurs at a rate of about 4.5%;
(2) maximum toxicity grade of the immune effector cell-associated neurotoxicity syndrome or associated symptom is Grade 1 or Grade 2, wherein optionally maximum toxicity grade of the immune effector cell-associated neurotoxicity syndrome or associated symptom is Grade 1, further wherein optionally the maximum toxicity grade of Grade 1 in the subject occurs at a rate of about 3.4%, or wherein optionally maximum toxicity grade of the immune effector cell-associated neurotoxicity syndrome or associated symptom is Grade 2, further wherein optionally the maximum toxicity grade of Grade 2 in the subject occurs at a rate of about 1.1%;
(3) time to onset of the immune effector cell-associated neurotoxicity syndrome or associated symptom ranges from about 6 to about 15 days, wherein optionally the time to the onset of the immune effector cell-associated neurotoxicity syndrome or associated symptom is at a median of about 9.5 days;
(4) duration of the immune effector cell-associated neurotoxicity syndrome or associated symptom ranges from about 1 to about 6 days, wherein optionally the duration of the immune effector cell-associated neurotoxicity syndrome or associated symptom is at a median of about 2 days; or
(5) the treatment comprises a corticosteroid and/or tocilizumab,
further wherein optionally the neurotoxicity is a CAR-T cell neurotoxicity, wherein optionally the CAR-T cell neurotoxicity in the subject occurs at a rate of about 17.0%, wherein optionally the CAR-T cell neurotoxicity comprises Grade 3/4 neurotoxicity, Grade 5 neurotoxicity, cranial nerve palsy, peripheral neuropathy, a movement and neurocognitive treatment-emergent adverse event, or any combination thereof, further wherein optionally:
(a) the CAR-T cell neurotoxicity is Grade 3/4 neurotoxicity that occurs in the subject at a rate of about 2.3%;
(b) the CAR-T cell neurotoxicity is Grade 5 neurotoxicity;
(c) the CAR-T cell neurotoxicity is cranial nerve palsy, wherein optionally the cranial nerve palsy in the subject occurs at a rate of about 9.1%, wherein optionally:
(i) the cranial nerve palsy is Grade 2 or Grade 3 cranial nerve palsy, further wherein optionally the cranial nerve palsy is Grade 2 cranial nerve palsy that occurs in the subject at a rate of about 8.0%, and further wherein optionally the cranial nerve palsy is Grade 3 cranial nerve palsy that occurs in the subject at a rate of about 1.1%;
(ii) time to onset of the cranial nerve palsy after administering the dose of the T cells to the subject ranges from about 17 days to about 60 days, further wherein optionally the time to onset of the cranial nerve palsy after administering the dose of the T cells to the subject is at a median of about 21 days;
(iii) the cranial nerve palsy affects cranial nerve III, V, or VII;
(iv) duration of the cranial nerve palsy ranges from about 15 days to about 262 days, further wherein optionally duration of the cranial nerve palsy is at a median of about 77 days; or
(v) the treatment comprises a corticosteroid;
(d) the CAR-T cell neurotoxicity is peripheral neuropathy, wherein optionally the peripheral neuropathy in the subject occurs at a rate of about 2.8%; or
(e) the CAR-T cell neurotoxicity is a movement and neurocognitive treatment-emergent adverse event, wherein optionally the movement and neurocognitive treatment-emergent adverse event is Grade 1, further wherein optionally the Grade 1 movement and neurocognitive treatment-emergent adverse event in the subject occurs at a rate of about 0.6%.

38. (canceled)

39. The method of claim 1, wherein:
(a) CD3+ cells comprising the CAR in the blood of the subject peak at a median of about 13 days after administering the T cells to the subject, wherein optionally the CD3+ cells comprising the CAR in the blood of the subject peak at a mean concentration of about 1523 cells/μL;
(b) CD3+ cells comprising the CAR in the blood of the subject remain detectable from about 13 days to about 631 days after administering the T cells to the subject, wherein optionally the CD3+ cells comprising the CAR in the blood of the subject remain detectable at a median of about 57 days after administering the T cells to the subject; or
(c) AUC₀₋₂₈ of CD3+ cells comprising the CAR in the blood of the subject is at a mean value of about 12,504 cells/μL.

40. The method of claim 1, wherein an extracellular antigen binding domain capable of specifically binding to an epitope of B-cell maturation antigen (BCMA) comprises a first and a second VHH domains, wherein the first VHH domain comprising a CDR1, a CDR2, and a CDR3 as set forth in the VHH domain comprising the amino acid sequence of SEQ ID NO: 2, and the second VHH domain comprising a CDR1, a CDR2, and a CDR3 as set forth in the VHH domain comprising the amino acid sequence of SEQ ID NO: 4, wherein optionally the first VHH domain comprises a CDR1 comprising the amino acid sequence of SEQ ID NO: 18, a CDR2 comprising the amino acid sequence of SEQ ID NO: 19, a CDR3 comprising the amino acid sequence of SEQ ID NO: 20, and the second VHH domain comprises a CDR1 comprising the amino acid sequence of SEQ ID NO: 21, a CDR2 comprising the amino acid sequence of SEQ ID NO: 22, and a CDR3 comprising the amino acid sequence of SEQ ID NO: 23, wherein optionally the first VHH domain comprises the amino acid sequence of SEQ ID NO: 2 and the second VHH domain comprises the amino acid sequence of SEQ ID NO: 4, wherein optionally the first VHH domain is at the N-terminus of the second VHH domain, or the first VHH domain is at the C-terminus of the second VHH domain, further wherein optionally:
(a) the first VHH domain is linked to the second VHH domain via a linker comprising the amino acid sequence of SEQ ID NO: 3;
(b) the transmembrane domain is derived from a molecule selected from the group consisting of CD8α, CD4, CD28, CD137, CD80, CD86, CD152 and PD1, wherein optionally the transmembrane domain is derived from CD8a and comprises the amino acid sequence of SEQ ID NO: 6;
(c) the intracellular signaling domain comprises a primary intracellular signaling domain of an immune effector cell, wherein optionally the primary intracellular signaling domain is derived from CD3ζ comprising the amino acid sequence of SEQ ID NO: 8;
(d) the intracellular signaling domain comprises a co-stimulatory signaling domain, wherein optionally the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 and any combination thereof, wherein optionally the co-stimulatory signaling domain comprises a cytoplasmic domain of CD137 comprising the amino acid sequence of SEQ ID NO: 7;
(e) the CAR further comprises a hinge domain located between the C-terminus of the extracellular antigen binding domain and the N-terminus of the transmembrane domain, wherein optionally the hinge domain is derived from CD8a comprising the amino acid sequence of SEQ ID NO: 5;
(f) the CAR further comprises a signal peptide located at the N-terminus of the polypeptide, wherein optionally the signal peptide is derived from CD8a comprising the amino acid sequence of SEQ ID NO: 1; or
(g) the CAR comprises the amino acid sequence of SEQ ID NO: 17;
(h) the dose of T cells is formulated in a composition comprising 5% dimethyl sulfoxide (DMSO);
(i) the administration of the dose of T cells reduces the risk of disease progression or death in the subject, further wherein optionally (1) the risk of disease progression or death is reduced relative to an administration of daratumumab-pomalidomide-dexamethasone (DPd) or pomalidomide-bortezomib-dexamethasone (PVd) treatment; (2) the risk of disease progression or death is reduced relative to an administration of ide-cel treatment; further wherein optionally the subject has an about 60% to about 75%, or about 74% reduced risk of disease progression or death,
(j) the IMiD is lenalidomide;
(k) the subject has received three or fewer, two or fewer, or only one prior line of therapy;
(l) the method is effective in obtaining an overall response rate (ORR) of about 75% to about 100%, further wherein optionally the ORR is about 84.6%;
(m) the treatment is effective in prolonging the median progression-free survival (PFS) time of the subject as compared to an administration of DPd or PVd treatment, further wherein optionally the PFS at 12 months after administration of the treatment is about 75.9%, further wherein optionally the PFS at 12 months after administration of DPd or PVd is about 48.6%;
(n) the treatment is more effective in obtaining a stringent complete response (sCR) in the subject as compared to an administration of DPd or PVd treatment, further wherein optimally the sCR after administration of the treatment is about 58.2%, further wherein optionally the sCR after administration of DPd or PVd is about 15.2%,
(o) the treatment is more effective in obtaining a very good partial response (VGPR) or better in the subject as compared to an administration of DPd or PVd treatment, further wherein optionally the VGPR or better after administration of the treatment is about 81.3%, further wherein optionally the VGPR or better after administration of DPd or PVd is about 45.5%.

41-61. (canceled)

62. The method of claim 40, wherein the method further comprises treating the subject for a CAR-T-associated adverse event after administering the dose of the T cells, wherein optionally the method comprises administering a treatment to the subject to alleviate the CAR-T-associated adverse event, wherein optionally the CAR-T-associated adverse event comprises a cytokine release syndrome (CRS) and/or neurotoxicity, wherein optionally:
(a) the CAR-T-associated adverse event is a CRS, further wherein optionally the CRS occurs in the subject at a rate of about 60% to about 90%, or a rate of about 76.1%, further wherein optionally maximum toxicity grade of the CRS is Grade 1, Grade 2 or Grade 3, further wherein optionally:
(1) the maximum toxicity grade of the CRS is Grade 1, wherein optionally the maximum toxicity grade of Grade 1 in the subject occurs at a rate of about 52.8%;
(2) the maximum toxicity grade of the CRS is Grade 2, wherein optionally the maximum toxicity grade of Grade 2 in the subject occurs at a rate of about 22.2%;
(3) the maximum toxicity grade of the CRS is Grade 3, wherein optionally the maximum toxicity grade of Grade 3 in the subject occurs at a rate of about 1.1%;
(4) time to first onset of the CRS ranges from about 1 to about 23 days, wherein optionally the time to the first onset of the CRS is at a median of about 8 days;
(5) duration of the CRS ranges from about 1 to about 17 days, wherein optionally the duration of the CRS is at a median of about 3 days; or
(6) the treatment comprises tocilizumab, oxygen, a corticosteroid, a vasopressor, or any combination thereof, or
(b) the CAR-T-associated adverse event is neurotoxicity, wherein optionally the neurotoxicity comprises an immune effector cell-associated neurotoxicity syndrome or associated symptom, movement and neurocognitive neurotoxicity, treatment-emergent adverse event of neurotoxicity, a non-immune effector cell-associated neurotoxicity syndrome or associated symptom, or any combination thereof, wherein optionally the neurotoxicity is an immune effector cell-associated neurotoxicity syndrome or associated symptom, further wherein optionally:
(1) the immune effector cell-associated neurotoxicity syndrome or associated symptom in the subject occurs at a rate of about 4.5%;
(2) maximum toxicity grade of the immune effector cell-associated neurotoxicity syndrome or associated symptom is Grade 1 or Grade 2, wherein optionally maximum toxicity grade of the immune effector cell-associated neurotoxicity syndrome or associated symptom is Grade 1, further wherein optionally the maximum toxicity grade of Grade 1 in the subject occurs at a rate of about 3.4%, or wherein optionally maximum toxicity grade of the immune effector cell-associated neurotoxicity syndrome or associated symptom is Grade 2, further wherein optionally the maximum toxicity grade of Grade 2 in the subject occurs at a rate of about 1.1%;
(3) time to onset of the immune effector cell-associated neurotoxicity syndrome or associated symptom ranges from about 6 to about 15 days, wherein optionally the time to the onset of the immune effector cell-associated neurotoxicity syndrome or associated symptom is at a median of about 9.5 days;
(4) duration of the immune effector cell-associated neurotoxicity syndrome or associated symptom ranges from about 1 to about 6 days, wherein optionally the duration of the immune effector cell-associated neurotoxicity syndrome or associated symptom is at a median of about 2 days; or
(5) the treatment comprises a corticosteroid and/or tocilizumab.

63. The method of claim 62, wherein the neurotoxicity is a CAR-T cell neurotoxicity, wherein optionally the CAR-T cell neurotoxicity in the subject occurs at a rate of about 17.0%, wherein optionally the CAR-T cell neurotoxicity comprises Grade 3/4 neurotoxicity, Grade 5 neurotoxicity, cranial nerve palsy, peripheral neuropathy, a movement and neurocognitive treatment-emergent adverse event, or any combination thereof, further wherein optionally:
(a) the CAR-T cell neurotoxicity is Grade 3/4 neurotoxicity that occurs in the subject at a rate of about 2.3%;
(b) the CAR-T cell neurotoxicity is Grade 5 neurotoxicity;
(c) the CAR-T cell neurotoxicity is cranial nerve palsy, wherein optionally the cranial nerve palsy in the subject occurs at a rate of about 9.1%, wherein optionally:
(1) the cranial nerve palsy is Grade 2 or Grade 3 cranial nerve palsy, further wherein optionally the cranial nerve palsy is Grade 2 cranial nerve palsy that occurs in the subject at a rate of about 8.0%, and further wherein optionally the cranial nerve palsy is Grade 3 cranial nerve palsy that occurs in the subject at a rate of about 1.1%;
(2) time to onset of the cranial nerve palsy after administering the dose of the T cells to the subject ranges from about 17 days to about 60 days, further wherein optionally the time to onset of the cranial nerve palsy after administering the dose of the T cells to the subject is at a median of about 21 days;
(3) the cranial nerve palsy affects cranial nerve III, V, or VII;
(4) duration of the cranial nerve palsy ranges from about 15 days to about 262 days, further wherein optionally duration of the cranial nerve palsy is at a median of about 77 days; or
(5) the treatment comprises a corticosteroid;
(d) the CAR-T cell neurotoxicity is peripheral neuropathy, wherein optionally the peripheral neuropathy in the subject occurs at a rate of about 2.8%; or
(e) the CAR-T cell neurotoxicity is a movement and neurocognitive treatment-emergent adverse event, wherein optionally the movement and neurocognitive treatment-emergent adverse event is Grade 1, further wherein optionally the Grade 1 movement and neurocognitive treatment-emergent adverse event in the subject occurs at a rate of about 0.6%.

64. The method of claim 40, wherein the subject has one or more high-risk cytogenetic abnormality selected from a group comprising Gain/amp(1q), del(17p), t(4;14), t(14;16), or any combination thereof, wherein optionally the subject has at least two cytogenetic abnormalities, further wherein optionally the subject has two, three, four, five, or more cytogenetic abnormalities, further wherein optionally the cytogenetic abnormality is a standard-risk cytogenetic abnormality.

65-66. (canceled)

67. The method of claim 1, wherein the administration of the dose of T cells is effective in obtaining a greater very good partial response (VGPR) or better in the subject as compared to an administration of DPd or PVd treatment, wherein optionally the VGPR or better after administration of the treatment is about 81.3%, wherein optionally the VGPR or better after administration of DPd or PVd is about 45.5%, wherein optionally the treatment is more effective in obtaining a stringent complete response (sCR) in the subject as compared to an administration of DPd or PVd treatment, further wherein optionally the sCR after administration of the treatment is about 58.2%, further wherein optionally the sCR after administration of DPd or PVd is about 15.2%, further wherein optionally the administration of the dose of T cells reduces the risk of disease progression or death in the subject, further wherein optionally the risk of disease progression or death is reduced relative to an administration of daratumumab-pomalidomide-dexamethasone (DPd) or pomalidomide-bortezomib-dexamethasone (PVd) treatment, further wherein optionally the risk of disease progression or death is reduced relative to an administration of ide-cel treatment, further wherein optionally the subject has an about 60% to about 75%, or about 74% reduced risk of disease progression or death.

68-77. (canceled)

78. The method of claim 67, wherein:
(1) the IMiD is lenalidomide,
(2) the subject has received three or fewer, two or fewer, or only one prior line of therapy;
(3) the method is effective in obtaining an overall response rate (ORR) of about 75% to about 100%, wherein optionally the ORR is about 84.6%; or
(4) the treatment is effective in prolonging the median progression-free survival (PFS) time of the subject as compared to an administration of DPd or PVd treatment, wherein optionally the PFS at 12 months after administration of the treatment is about 75.9%, wherein optionally the PFS at 12 months after administration of DPd or PVd is about 48.6%.

79-86. (canceled)

87. The method of claim 67, wherein the method further comprises treating the subject for a CAR-T-associated adverse event after administering the dose of the T cells, wherein optionally the method comprises administering a treatment to the subject to alleviate the CAR-T-associated adverse event, wherein optionally the CAR-T-associated adverse event comprises a cytokine release syndrome (CRS) and/or neurotoxicity, wherein optionally:
(a) the CAR-T-associated adverse event is a CRS, further wherein optionally the CRS occurs in the subject at a rate of about 60% to about 90%, or a rate of about 76.1%, further wherein optionally maximum toxicity grade of the CRS is Grade 1, Grade 2 or Grade 3, further wherein optionally:
(1) the maximum toxicity grade of the CRS is Grade 1, wherein optionally the maximum toxicity grade of Grade 1 in the subject occurs at a rate of about 52.8%;
(2) the maximum toxicity grade of the CRS is Grade 2, wherein optionally the maximum toxicity grade of Grade 2 in the subject occurs at a rate of about 22.2%;
(3) the maximum toxicity grade of the CRS is Grade 3, wherein optionally the maximum toxicity grade of Grade 3 in the subject occurs at a rate of about 1.1%;
(4) time to first onset of the CRS ranges from about 1 to about 23 days, wherein optionally the time to the first onset of the CRS is at a median of about 8 days;
(5) duration of the CRS ranges from about 1 to about 17 days, wherein optionally the duration of the CRS is at a median of about 3 days; or
(6) the treatment comprises tocilizumab, oxygen, a corticosteroid, a vasopressor, or any combination thereof, or
(b) the CAR-T-associated adverse event is neurotoxicity, wherein optionally the neurotoxicity comprises an immune effector cell-associated neurotoxicity syndrome or associated symptom, movement and neurocognitive neurotoxicity, treatment-emergent adverse event of neurotoxicity, a non-immune effector cell-associated neurotoxicity syndrome or associated symptom, or any combination thereof, wherein optionally the neurotoxicity is an immune effector cell-associated neurotoxicity syndrome or associated symptom, further wherein optionally:
(1) the immune effector cell-associated neurotoxicity syndrome or associated symptom in the subject occurs at a rate of about 4.5%;
(2) maximum toxicity grade of the immune effector cell-associated neurotoxicity syndrome or associated symptom is Grade 1 or Grade 2, wherein optionally maximum toxicity grade of the immune effector cell-associated neurotoxicity syndrome or associated symptom is Grade 1, further wherein optionally the maximum toxicity grade of Grade 1 in the subject occurs at a rate of about 3.4%, or wherein optionally maximum toxicity grade of the immune effector cell-associated neurotoxicity syndrome or associated symptom is Grade 2, further wherein optionally the maximum toxicity grade of Grade 2 in the subject occurs at a rate of about 1.1%;
(3) time to onset of the immune effector cell-associated neurotoxicity syndrome or associated symptom ranges from about 6 to about 15 days, wherein optionally the time to the onset of the immune effector cell-associated neurotoxicity syndrome or associated symptom is at a median of about 9.5 days;
(4) duration of the immune effector cell-associated neurotoxicity syndrome or associated symptom ranges from about 1 to about 6 days, wherein optionally the duration of the immune effector cell-associated neurotoxicity syndrome or associated symptom is at a median of about 2 days; or
(5) the treatment comprises a corticosteroid and/or tocilizumab.

88. The method of claim 87, wherein the neurotoxicity is a CAR-T cell neurotoxicity, wherein optionally the CAR-T cell neurotoxicity in the subject occurs at a rate of about 17.0%, wherein optionally the CAR-T cell neurotoxicity comprises Grade 3/4 neurotoxicity, Grade 5 neurotoxicity, cranial nerve palsy, peripheral neuropathy, a movement and neurocognitive treatment-emergent adverse event, or any combination thereof, further wherein optionally:
(a) the CAR-T cell neurotoxicity is Grade 3/4 neurotoxicity that occurs in the subject at a rate of about 2.3%;
(b) the CAR-T cell neurotoxicity is Grade 5 neurotoxicity;
(c) the CAR-T cell neurotoxicity is cranial nerve palsy, wherein optionally the cranial nerve palsy in the subject occurs at a rate of about 9.1%, wherein optionally:
(1) the cranial nerve palsy is Grade 2 or Grade 3 cranial nerve palsy, further wherein optionally the cranial nerve palsy is Grade 2 cranial nerve palsy that occurs in the subject at a rate of about 8.0%, and further wherein optionally the cranial nerve palsy is Grade 3 cranial nerve palsy that occurs in the subject at a rate of about 1.1%;
(2) time to onset of the cranial nerve palsy after administering the dose of the T cells to the subject ranges from about 17 days to about 60 days, further wherein optionally the time to onset of the cranial nerve palsy after administering the dose of the T cells to the subject is at a median of about 21 days;
(3) the cranial nerve palsy affects cranial nerve III, V, or VII;
(4) duration of the cranial nerve palsy ranges from about 15 days to about 262 days, further wherein optionally duration of the cranial nerve palsy is at a median of about 77 days; or
(5) the treatment comprises a corticosteroid;
(d) the CAR-T cell neurotoxicity is peripheral neuropathy, wherein optionally the peripheral neuropathy in the subject occurs at a rate of about 2.8%; or
(e) the CAR-T cell neurotoxicity is a movement and neurocognitive treatment-emergent adverse event, wherein optionally the movement and neurocognitive treatment-emergent adverse event is Grade 1, further wherein optionally the Grade 1 movement and neurocognitive treatment-emergent adverse event in the subject occurs at a rate of about 0.6%.

89. The method of claim 67, wherein the subject has one or more high-risk cytogenetic abnormality selected from a group comprising Gain/amp(1q), del(17p), t(4;14), t(14;16), or any combination thereof, wherein optionally the subject has at least two cytogenetic abnormalities, wherein optionally the subject has two, three, four, five, or more cytogenetic abnormalities, and wherein optionally the cytogenetic abnormality is a standard-risk cytogenetic abnormality.

90-91. (canceled)


SEQUENCE LISTING (23 sequences)

SEQ ID NO: 1 - protein, 21 aa, synthetic construct
  source 1..21: note = Ciltacabtagene autoleucel CAR CD8alpha signal peptide, CD8alpha SP amino acid sequence
MALPVTALLLPLALLLHAARP

SEQ ID NO: 2 - protein, 119 aa, synthetic construct
  source 1..119: note = Ciltacabtagene autoleucel CAR BCMA binding domain, VHH1 amino acid sequence
QVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSS

SEQ ID NO: 3 - protein, 5 aa, synthetic construct
  source 1..5: note = Ciltacabtagene autoleucel CAR BCMA binding domain, G4S linker amino acid sequence
GGGGS

SEQ ID NO: 4 - protein, 118 aa, synthetic construct
  source 1..118: note = Ciltacabtagene autoleucel CAR BCMA binding domain, VHH2 amino acid sequence
EVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSS

SEQ ID NO: 5 - protein, 45 aa, synthetic construct
  source 1..45: note = Ciltacabtagene autoleucel CAR CD8alpha hinge amino acid sequence
TTTPAPRPPTPAPTIASQPLSLRPEACRPAAGGAVHTRGLDFACD

SEQ ID NO: 6 - protein, 24 aa, synthetic construct
  source 1..24: note = Ciltacabtagene autoleucel CAR CD8alpha transmembrane amino acid sequence
IYIWAPLAGTCGVLLLSLVITLYC

SEQ ID NO: 7 - protein, 42 aa, synthetic construct
  source 1..42: note = Ciltacabtagene autoleucel CAR CD137 Cytoplasmic amino acid sequence
KRGRKKLLYIFKQPFMRPVQTTQEEDGCSCRFPEEEEGGCEL

SEQ ID NO: 8 - protein, 112 aa, synthetic construct
  source 1..112: note = Ciltacabtagene autoleucel CAR CD3zeta Cytoplasmic amino acid sequence
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - DNA, 63 bp, synthetic construct
  source 1..63: mol_type = other DNA; note = Ciltacabtagene autoleucel CAR CD8alpha signal peptide CD8alpha SP nucleic acid sequence
atggctctgcccgtcaccgctctgctgctgcctctggctctgctgctgcacgctgctcgccct

SEQ ID NO: 10 - DNA, 357 bp, synthetic construct
  source 1..357: mol_type = other DNA; note = Ciltacabtagene autoleucel CAR BCMA binding domain, VHH1 nucleic acid sequence
caggtcaaactggaagaatctggcggaggcctggtgcaggcaggacggagcctgcgcctgagctgcgcagcatccgagcacaccttcagctcccacgtgatgggctggtttcggcaggccccaggcaaggagagagagagcgtggccgtgatcggctggagggacatctccacatcttacgccgattccgtgaagggccggttcaccatcagccgggacaacgccaagaagacactgtatctgcagatgaacagcctgaagcccgaggacaccgccgtgtactattgcgcagcaaggagaatcgacgcagcagactttgattcctggggccagggcacccaggtgacagtgtctagc

SEQ ID NO: 11 - DNA, 15 bp, synthetic construct
  source 1..15: mol_type = other DNA; note = Ciltacabtagene autoleucel CAR BCMA binding domain, G4S linker nucleic acid sequence
ggaggaggaggatct

SEQ ID NO: 12 - DNA, 354 bp, synthetic construct
  source 1..354: mol_type = other DNA; note = Ciltacabtagene autoleucel CAR BCMA binding domain, VHH2 nucleic acid sequence
gaggtgcagctggtggagagcggaggcggcctggtgcaggccggaggctctctgaggctgagctgtgcagcatccggaagaaccttcacaatgggctggtttaggcaggcaccaggaaaggagagggagttcgtggcagcaatcagcctgtcccctaccctggcctactatgccgagagcgtgaagggcaggtttaccatctcccgcgataacgccaagaatacagtggtgctgcagatgaactccctgaaacctgaggacacagccctgtactattgtgccgccgatcggaagagcgtgatgagcattagaccagactattgggggcagggaacacaggtgaccgtgagcagc

SEQ ID NO: 13 - DNA, 135 bp, synthetic construct
  source 1..135: mol_type = other DNA; note = Ciltacabtagene autoleucel CAR CD8alpha hinge nucleic acid sequence
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 14 - DNA, 72 bp, synthetic construct
  source 1..72: mol_type = other DNA; note = Ciltacabtagene autoleucel CAR CD8alpha transmembrane nucleic acid sequence
atctacatctgggcgcccttggccgggacttgtggggtccttctcctgtcactggttatcaccctttactgc

SEQ ID NO: 15 - DNA, 126 bp, synthetic construct
  source 1..126: mol_type = other DNA; note = Ciltacabtagene autoleucel CAR CD137 Cytoplasmic nucleic acid sequence
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 16 - DNA, 339 bp, synthetic construct
  source 1..339: mol_type = other DNA; note = Ciltacabtagene autoleucel CAR CD3z Cytoplasmic nucleic acid sequence
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 17 - protein, 488 aa, synthetic construct
  source 1..488: note = Ciltacabtagene autoleucel CAR amino acid sequence
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 5 aa, synthetic construct
  source 1..5: note = Ciltacabtagene autoleucel CAR BCMA binding domain, VHH1 CDR1
SHVMG

SEQ ID NO: 19 - protein, 17 aa, synthetic construct
  source 1..17: note = Ciltacabtagene autoleucel CAR BCMA binding domain, VHH1 CDR2
VIGWRDISTSYADSVKG

SEQ ID NO: 20 - protein, 11 aa, synthetic construct
  source 1..11: note = Ciltacabtagene autoleucel CAR BCMA binding domain, VHH1 CDR3
ARRIDAADFDS

SEQ ID NO: 21 - protein, 5 aa, synthetic construct
  source 1..5: note = Ciltacabtagene autoleucel CAR BCMA binding domain, VHH2 CDR1
TFTMG

SEQ ID NO: 22 - protein, 17 aa, synthetic construct
  source 1..17: note = Ciltacabtagene autoleucel CAR BCMA binding domain, VHH2 CDR2
AISLSPTLAYYAESVKG

SEQ ID NO: 23 - protein, 13 aa, synthetic construct
  source 1..13: note = Ciltacabtagene autoleucel CAR BCMA binding domain, VHH2 CDR3
ADRKSVMSIRPDY